(19) United States
(12) Patent Application Publication
(10) Pub. No.: US 2024/0090908 A1
(43) Pub. Date: Mar. 21, 2024

(54) DEVICE FOR MICROLITER-SCALE LYMPHATIC DELIVERY OF CORONAVIRUS VACCINES AND METHODS OF USE THEREOF

(71) Applicant: Sorrento Therapeutics, Inc., San Diego, CA (US)

(72) Inventors: Russell F. Ross, San Diego, CA (US); Henry Hongjun Ji, San Diego, CA (US)

(21) Appl. No.: 18/262,557

(22) PCT Filed: Jan. 21, 2022
(86) PCT No.: PCT/US2022/013363

§ 371 Date: Jul. 21, 2023

Related U.S. Application Data
(60) Provisional application No. 63/140,670, filed on Jan. 22, 2021.

(51) Int. Cl. A61B 17/20; A61M 37/00

(52) U.S. Cl. CPC A61B 17/205; A61M 37/0015; A61M 2037/0023; A61M 2037/003

(57) ABSTRACT

A fluid delivery device adapted for microliter-scale injections of coronavirus vaccines across a dermal barrier of a patient, for example to the lymphatic system of the patient, is described. The present disclosure also provides methods of administering coronavirus vaccines across a dermal barrier of a patient, for example to the lymphatic system of a patient.

### RELATED APPLICATION

This application claims the benefit of U.S. Provisional Patent Application No. 63/140,670 filed Jan. 22, 2021, the contents of which are herein incorporated by reference in their entirety.

### TECHNICAL FIELD

The present disclosure relates generally to a fluid delivery device, and more specifically relates to a fluid delivery device adapted for microliter-scale injections (herein referred to as a “microdose device”). The present disclosure also relates to methods of applying a fluid delivery device to a patient's skin to deliver a fluidic composition across a dermal barrier of the patient, for example to the lymphatic system of the patient. The present disclosure relates more particularly to a fluid delivery device adapted for microliter-scale injections of coronavirus vaccines across a dermal barrier of a patient, for example to the lymphatic system of the patient. The present disclosure also relates to methods of administering coronavirus vaccines across a dermal barrier of a patient, for example to the lymphatic system of a patient.

### BACKGROUND

Coronaviruses is a group of viruses that causes diseases in birds, mammals and humans. The diseases include respiratory infections and enteric infections which can be mild or lethal. Coronaviruses are viruses in the subfamily Orthocoronavirinae, in the family Coronaviridae, in the order Nidovirales. The genus Coronavirus includes avian infectious bronchitis virus, bovine coronavirus, canine coronavirus, human coronavirus 299E, human coronavirus OC43, murine hepatitis virus, rat coronavirus, and porcine hemagglutinating encephalomyelitis virus. The genus Torovirus includes Berne virus and Breda virus. Coronaviruses are enveloped viruses having a positive-sense single-stranded RNA genome and a nucleocapsid of helical symmetry. The genomic size of coronaviruses ranges from approximately 26 to 32 kilobases, which is believed to be the largest for an RNA virus.

The name “coronavirus” is derived from the Latin corona and the Greek korone (e.g., “garland” or “wreath”), meaning crown or halo. The corona reference relates to the characteristic appearance of virions (the infective form of the virus) by electron microscopy, which have a fringe of large, bulbous surface projections creating an image reminiscent of a royal crown or of the solar corona. This morphology is created by the viral spike (S) peplomers, which are proteins that populate the surface of the virus and determine host tropism. Proteins that contribute to the overall structure of all coronaviruses are the spike (S), envelope (E), membrane (M) and nucleocapsid (N). In the specific case of the SARS coronavirus, a defined receptor-binding domain on S mediates the attachment of the virus to its cellular receptor, angiotensin-converting enzyme 2 (ACE2). Some coronaviruses (specifically the members of Betacoronavirus subgroup A) also have a shorter spike-like protein called hemagglutinin esterase (HE). The 2019-2020 China pneumonia outbreak in Wuhan was traced to a novel coronavirus, labeled 2019-nCoV by the World Health Organization (WHO) and is also known as SARS-CoV-2.

There is a need in the art for methods for preventing or treating coronavirus infections in human and animal patients. Accordingly, the embodiments described herein are provided in an effort to meet this need and/or provide other benefits, or at least provide the public with a useful choice.

### SUMMARY

According to a first aspect, the present disclosure provides a device (herein also referred to as a “microdose device”) configured for delivering a fluidic composition across a dermal barrier of a patient. The device comprises a microneedle fluidic block assembly, comprising: a microneedle array comprising a plurality of microneedles disposed on a distal face of a base plate, wherein the microneedles have a fluidic exit channel defined therein, the microneedles capable of penetrating the stratum corneum of the skin of a patient and delivering a fluidic composition to a depth below the surface of the skin of the patient. The device also comprises a fluidic distribution block having a distal face coupled to a proximal face of the base plate of the microneedle array, the fluidic distribution block comprising a fluid distribution manifold defined therein and configured to be fluidically connected with the fluidic exit channels of the microneedles and to controllably distribute the fluidic composition to the plurality of microneedles through the fluidic exit channels. The device also comprises a syringe connection assembly having a fluidic path defined therein, the syringe connection assembly comprising: a distal end coupled to a proximal face of the fluidic distribution block, the fluidic path of the syringe connection assembly fluidically connected to the fluid distribution manifold, and a proximal end configured to be coupled to a syringe barrel having a bore defined therein, the fluidic path of the syringe connection assembly configured to be fluidically connected to the bore of the syringe barrel.

The device can further include the following details, which can be combined with one another in any combinations unless clearly mutually exclusive:

(i) The syringe connection assembly may comprise a plenum coupled to and fluidically connected with a tubing connector. The tubing connector may have a distal portion coupled to a proximal face of the plenum and a proximal portion configured to be fluidically connected to the bore of the syringe barrel. The plenum may have a distal face coupled to the proximal face of the fluidic distribution block, and fluidically connected to the fluid distribution manifold.

(ii) The device may further comprise a first gasket disposed between and coupled to the distal end of the syringe connection assembly and the proximal face of the fluidic distribution block. The first gasket may include a hole in fluidic connection with the fluidic path of the syringe connection assembly and the fluid distribution manifold.

(iii) The first gasket may have a proximal face and a distal face, wherein the proximal face and the distal face has an adhesive layer disposed thereon and adapted to adhere the distal end of the syringe connection assembly to the proximal face of the fluidic distribution block.

(iv) The fluid distribution manifold may be configured to provide a substantially equal flow rate of the fluidic composition to the exit channels in each microneedle.

(v) The fluid distribution manifold may comprise: a proximal entrance disposed within the proximal face of the fluidic distribution block and in fluidic connection with the distal end of the syringe connection assembly; supply channels fluidically connected to the proximal entrance and configured to distribute a fluidic composition to a plurality of resistance channels; the plurality of resistance channels fluidically connected to the supply channels and configured to provide a resistance to flow of the fluidic composition; a plurality of outlet apertures, each outlet aperture fluidically connected to a resistance channel and a fluidic exit channel.

(vi) The fluidic distribution block may comprise a proximal portion having a distal face coupled to a proximal face of a distal portion, wherein the supply channels and the resistance channels are disposed on the distal face of the proximal portion and/or the proximal face of the distal portion.

(vii) The fluidic distribution block may comprise a polymer material, a glass material and/or a silicon material, and the fluid distribution manifold may be formed therein by a drilling method, a cutting method, a powder blasting method, an etching method, or any combinations thereof.

(viii) The proximal portion of the fluidic distribution block and the distal portion of the fluidic distribution block may be bonded together.

(ix) The resistance channels may have: a length of from 400 m to 1,000 μm; an axial depth of from 10 μm to about 20 μm; and a lateral width of from 15 μm to 70 μm.

(x) The plurality of microneedles may be from 2 to 100 microneedles.

(xi) Each of the resistance channels may include one or more inlet apertures adapted to be in fluidic connection with the supply channel. The resistance channels may comprise inner resistance channels located proximal to a lateral center of the fluidic distribution block, and outer resistance channels located distal to the lateral center of the fluidic distribution block. Two or more inner resistance channels may be in fluidic connection with one inlet aperture; and each outer resistance channel may be in fluidic connection with one inlet aperture.

(xii) The device may further comprise a protective cap coupled to the distal end of the syringe connection assembly and configured to protect the physical integrity and/or sterility of the microneedle fluidic block assembly.

(xiii) The protective cap may be configured to be slidably coupled to the syringe connection assembly.

(xiv) The device may further comprise a syringe including a barrel, wherein the proximal end of the syringe connection assembly may be coupled to the syringe barrel and fluidically connected to the bore of the syringe barrel.

(xv) The syringe may further comprise a plunger slidably disposed within a longitudinal axis of the bore, the syringe adapted to eject a volume of from 1 μl to 500 μl of a fluidic composition disposed within the bore in response to an axial force applied to the plunger.

(xvi) The syringe may be adapted to eject the volume of the fluidic composition over a period of time from 0.1 second to 300 seconds.

(xvii) The syringe may further comprise a fluidic composition disposed within the bore.

(xviii) In response to an axial force applied to the plunger, the device may be adapted to deliver the fluidic composition to a patient through the exit channels of the plurality of microneedles.

(xix) The device may be adapted to be manually operable by a user, wherein the axial force is applied by the hand of the user.

(xx) The microneedles further comprise a nanotopography.

(xxi) The microneedles may have an axial length of from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm.

(xxii) The fluidic composition may comprise a coronavirus vaccine.

(xxiii) The coronavirus vaccine may be an inactivated virus vaccine, a live-virus vaccine, a recombinant protein vaccines, a vectored vaccine, an RNA vaccine or a DNA vaccine.

(xxiv) The coronavirus vaccine may be a SARS-CoV-2 vaccine.

(xxv) The SARS-CoV-2 vaccine may comprise a recombinant fusion protein comprising a coronavirus spike S1 protein or a fragment thereof linked to an immunoglobulin Fc region or a fragment thereof.

According to a second aspect, the present disclosure provides a method of delivering a fluidic composition across a dermal barrier of a patient. The method comprises inserting a plurality of the microneedles of the device of the present disclosure across the dermal barrier of the patient; and delivering a fluidic composition through the fluidic exit channels of the plurality of microneedles to a location below the dermal barrier.

The method can further include the following details, which can be combined with one another in any combinations unless clearly mutually exclusive:

(i) The plurality of microneedles may be from 2 to 100 microneedles.

(ii) A total volume of the fluidic composition delivered may be from 1 μL to 500 μL.

(iii) The fluidic composition may be delivered to the patient at a rate of up to 20 μL, 19 μL, 18 μL, 17 μL, 16 μL, 15 μL, 14 μL, 13 μL, 12 μL, 11 μL, 10 μL, 9 μL, 8 μL, 7 μL, 6 μL, 5 μL, 4 μL, 3 μL, 2 μL, 1 μL, 0.5 μL, 0.1 μL, or 0.01 μL per second per microneedle.

(iv) The method may further comprise transporting the fluidic composition to the lymphatic system of the patient.

(v) The proximal end of the syringe connection assembly may be coupled to the syringe barrel and fluidically connected to the bore of the syringe barrel; the syringe may further comprise a plunger slidably disposed within the longitudinal axis of the bore, the syringe may be adapted to eject a volume of from 1 μl to 500 μl of a fluidic composition disposed within the bore in response to an axial force applied to the plunger; and the syringe may comprise a fluidic composition disposed within the bore. The method may comprise: placing the plurality of microneedles on the skin of the patient proximate to a first position under the skin of the patient, wherein the first position is proximate to lymph vessels and/or lymph capillaries in the patient's lymphatic system; inserting the plurality of microneedles into the patient to a depth whereby at least the epidermis is penetrated and an end of at least one of the microneedles is proximate to the first position; and delivering a volume of the fluidic composition from the device via the plurality of microneedles to the first position in response to applying an axial force to the plunger of the syringe.

(vi) The inserting the plurality of microneedles into the patient may be to a depth from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm.

(vii) The fluidic composition may be delivered to a lymph node, a lymph vessel, an organ that is part of the lymphatic system or a combination thereof.

(viii) The lymph node may be selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, splenic lymph nodes, and any combinations thereof.

(ix) The fluidic composition may comprise a coronavirus vaccine.

(x) The coronavirus vaccine may be an inactivated virus vaccine, a live-virus vaccine, a recombinant protein vaccines, a vectored vaccine, an RNA vaccine or a DNA vaccine.

(xi) The coronavirus vaccine may be a SARS-CoV-2 vaccine.

(xii) The SARS-CoV-2 vaccine may comprise a recombinant fusion protein comprising a coronavirus spike S1 protein or a fragment thereof linked to an immunoglobulin Fc region or a fragment thereof.

(xiii) The method may provide delivery of from about 10 to 40 times higher concentration of the coronavirus vaccine to lymph nodes in the patient compared to intravenous, subcutaneous, intramuscular, or intradermal routes of administration.

(xiv) The method may provide up to 1.5-fold, 2-fold, 3-fold, 4-fold, 5-fold, 6-fold, 7-fold, 8-fold, 9-fold, or 10-fold increased level of Th1 (e.g. CD4⁺IFNγ⁺) T-cells and Th2 (e.g. CD4⁺ IL-4⁺) T-cells in a patient as compared to other routes of administration such as such as intravenous, subcutaneous, intramuscular, or intradermal injections of the same coronavirus vaccine given to a patient at a same dose.

(xv) The method may provide up to 1.5-fold, 2-fold, 3-fold, 4-fold, 5-fold, 6-fold, 7-fold, 8-fold, 9-fold, or 10-fold increased ratio of Th1 response to Th2 response (e.g. an increased ratio of CD4⁺IFNγ⁺ T-cells to CD4⁺ IL-4⁺ T-cells) in a patient as compared to other routes of administration such as such as intravenous, subcutaneous, intramuscular, or intradermal injections of the same coronavirus vaccine given to a patient at a same dose.

(xvi) The method may provide an increase in Th1 T-cells that is up to 1.5-fold, 2-fold, 3-fold, 4-fold, 5-fold, 6-fold, 7-fold, 8-fold, 9-fold, or 10-fold higher than an increase in Th2 T-cells.

(xvii) The method may provide up to 1.5-fold, 2-fold, 3-fold, 4-fold, 5-fold, 6-fold, 7-fold, 8-fold, 9-fold, or 10-fold increase in CD8⁺ (e.g. CD8⁺IFNγ⁺) T-cells in a patient as compared to other routes of administration such as such as intravenous, subcutaneous, intramuscular, or intradermal injections of the same coronavirus vaccine given to a patient at a same dose.

(xviii) The coronavirus vaccine may be at a concentration of up to 10 mg/mL, 9 mg/mL, 8 mg/mL, 7 mg/mL, 6 mg/mL, 5 mg/mL, 4 mg/mL, 3 mg/mL, 2 mg/mL, or 1 mg/mL.

(xix) The device may be manually operated by a user, and the axial force may be applied by the hand of the user.

### BRIEF DESCRIPTION OF THE DRAWINGS

For a more complete understanding of the present disclosure and the associated features and advantages, reference is now made to the following description, taken in conjunction with the accompanying drawings, which are not to scale, and in which:

FIG. 1A is a perspective exploded view schematic of an example first set of components adapted to be combined to form a first example microdose device 10.

FIG. 1B is a perspective view schematic of the example first set of components of FIG. 1A combined to form a first example microdose device 10.

FIG. 1C is aside view of the example first set of components of FIG. 1B combined to form a first example microdose device 10.

FIG. 1D is a schematic of a cross-section view at plane B-B of FIG. 1C of the example first microdose device 10.

FIG. 1E is an axial plan top-down view schematic of a detail of the first example microdose device 10.

FIG. 2A is a perspective view schematic of an example microdose device 10 including a protective cap 160 attached, and a syringe 20.

FIG. 2B is a perspective view schematic of an example microdose device 10 attached to a syringe 20, with the microdose device 10 including the attached protective cap 160.

FIG. 2C is a perspective view schematic of an example microdose device 10, with protective cap 160 removed, attached to a syringe 20.

FIG. 2D is a side view schematic of an example microdose device 10 attached to a syringe 20, with the microdose device 10 including the protective cap 160.

FIG. 2E is a side view schematic of an example microdose device 10, with protective cap 160 removed, attached to a syringe 20.

FIG. 2F is a side view schematic of two example distal ends 212 of barrels 210 of syringes 20 adapted to couple to a tubing connector 120 of a microdose device 10.

FIG. 3A is a perspective view schematic of an example microneedle fluidic block assembly 150.

FIG. 3B is an exploded perspective view schematic of an example microneedle fluidic block assembly 150 of FIG. 3A, including a fluidic distribution block 650 comprising a proximal layer 650a and a distal layer 650b, and a microneedle array 660.

FIG. 3C is an axial plan top-down schematic of an example microneedle fluidic block assembly 150 showing a view of a plurality of microneedles 156 disposed on a base plate 300.

FIG. 3D is a side view schematic of an example microneedle fluidic block assembly 150.

FIG. 3E is a view of detail A of FIG. 3D.

FIG. 3F is another side view schematic of an example microneedle fluidic block assembly 150.

FIG. 3G is an axial plan view schematic through an example microneedle fluidic block assembly 150, including an example microneedle array 660 and example proximal layer 650a and distal layer 650b of an example fluidic distribution block 650.

FIG. 3H is an axial plan view schematic of an example distal layer 650b of a fluidic distribution block 650 of an example microneedle fluidic block assembly 150.

FIG. 3I is a view of detail B of FIG. 3H.

FIG. 3J is view of detail C of FIG. 3H.

FIG. 3K is a side view schematic of a proximal layer 650a and a distal layer 650b of an example fluidic distribution block 650.

FIG. 3L is an axial plan view schematic through an example proximal layer 650a and distal layer 650b of an example fluidic distribution block 650.

FIG. 3M is a Table of example parameters of example device designs having the indicated combinations of resistance channel length, resistance channel depth, and resistance channel width, and associated estimated fluid flow rate per microneedle at 0.7 bar pressure, and estimated resistance ratio (Rchannel/Rmicroneedle) based on a microneedle having an exit channel having a length 400 μm and a width 40 μm.

FIG. 3N is a Table of example parameters of example device designs having the indicated combinations of resistance channel length, resistance channel depth, and resistance channel width, and associated estimated fluid flow rate per microneedle at 1.0 bar pressure, and estimated resistance ratio (Rchannel/Rmicroneedle) based on a microneedle having an exit channel having a length 400 μm and a width 40 μm.

FIG. 4A is a perspective view schematic of an example plenum 130.

FIG. 4B is an axial plan top-down view of the example plenum 130 of FIG. 4A.

FIG. 4C is an example schematic of a cross-section view at plane A-A of FIG. 4B.

FIG. 4D is a side view schematic of the example plenum 130 of FIG. 4A.

FIG. 4E is an axial plan bottom-up view schematic of the example plenum 130 of FIG. 4A.

FIG. 5A is an axial plan top-down view schematic of an example protective cap 160.

FIG. 5B is a side view schematic of an example protective cap 160.

FIG. 5C is an axial plan bottom-up view schematic of an example protective cap 160.

FIG. 5D is an example schematic of a cross-section view at plane A-A of FIG. 5B.

FIG. 5E is a view of detail B of FIG. 5D.

FIG. 6A is a perspective exploded view schematic of an example second set of components adapted to be combined to form a microdose device 10.

FIG. 6B is a perspective view schematic of the example second set of components of FIG. 6A combined to form an example microdose device 10.

FIG. 6C is a top-down view schematic of an example microdose device 10.

FIG. 6D is an example schematic of a cross-section view at plane A-A of FIG. 6C.

FIG. 6E is a view of detail B of FIG. 6D.

FIG. 7A is a perspective view schematic of combined proximal layer 650a and distal layer 650b of an example fluidic distribution block 650.

FIG. 7B is an axial plan view schematic of combined proximal layer 650a and distal layer 650b of the example fluidic distribution block 650 of FIG. 7A.

FIG. 7C is a view of detail B of FIG. 7B.

FIG. 7D is a schematic of a cross-section view at plane C-C of FIG. 7C.

FIG. 7E is a view of detail D of FIG. 7D.

FIG. 7F is a side view schematic of the combined proximal layer 650a and distal layer 650b of the example fluidic distribution block 650 of FIG. 7A.

FIG. 7G is a view of detail A of FIG. 7F.

FIG. 8A is a perspective view schematic of an example microneedle array 660.

FIG. 8B is an axial plan view schematic of the example microneedle array 660 of FIG. 8A.

FIG. 8C is view of detail B of FIG. 8B.

FIG. 8D is a side view schematic of the example microneedle array 660 of FIG. 8A.

FIG. 8E is a view of detail A of FIG. 8D.

FIG. 9A is a perspective view schematic of an example draped microneedle array 900.

FIG. 9B is an exploded perspective view schematic of an example microneedle array 660, third gasket 1000 and film 1100 adapted to be combined to form the example draped microneedle array 900 of FIG. 9A.

FIG. 9C is an axial plan top-down view schematic of an example draped microneedle array 900.

FIG. 9D is a side view schematic of an example draped microneedle array 900.

FIG. 9E is a view of detail A of FIG. 9D.

FIG. 10A is a perspective view schematic of combined proximal layer 650a and distal layer 650b of an example fluidic distribution block 650.

FIG. 10B is an axial plan view schematic of combined proximal layer 650a and distal layer 650b of the example fluidic distribution block 650 of FIG. 10A.

FIG. 10C is a view of detail A of FIG. 10B.

FIG. 10D is a schematic of a cross-section view at plane B-B of FIG. 10C.

FIG. 10E is a view of detail C of FIG. 10D.

FIG. 10F is a view of detail D of FIG. 10D.

FIG. 10G is a side view schematic of the combined proximal layer 650a and distal layer 650b of the example fluidic distribution block 650 of FIG. 10A.

FIG. 10H is a view of detail E of FIG. 10G.

FIG. 11 is an example near-infrared fluorescence (NIRF) image of indocyanine green (ICG) in lymphatics of mice following injection of ICG using an example microdose device as described herein coupled to a syringe as described herein.

FIG. 12 is a schematic showing an example time course of near-infrared fluorescence (NIRF) imaging of lymphatic delivery of ICG in mice to right brachial lymph node.

FIG. 13A is a graph reporting example S1-specific serum IgG antibody optical density (OD, 450 nm) in mice following intramuscular injection of rS1-Fc vaccine.

FIG. 13B is a graph reporting example S1-specific serum IgG antibody optical density (OD, 450 nm) in mice following intradermal injection of rS1-Fc vaccine.

FIG. 13C is a graph reporting example S1-specific serum IgG antibody optical density (OD, 450 nm) in mice following SOFUSA® DoseConnect™ administration of rS1-Fc vaccine.

FIG. 14A is a graph showing box and whisker plots reporting example fold increases in T-cell responses (Th1 and Th2) in mice following administration of rS1-Fc vaccine via SOFUSA® DoseConnect™, or intramuscular or intradermal injection.

FIG. 14B is a graph showing box and whisker plots reporting example fold increases in T-cell responses (CD8⁺IFNγ⁺) in mice following administration of rS1-Fc vaccine via SOFUSA® DoseConnect™, or intramuscular or intradermal injection.

FIG. 14C is graphs reporting example results of flow cytometry analysis of T-cells from plasma of mice following administration of rS1-Fc vaccine via SOFUSA® DoseConnect™.

FIG. 14D is a graph showing box and whisker plots reporting example Mean Fluorescence Intensity (MFI) quantification by flow cytometry of CD4⁺IFNγ⁺ T-cells, CD4+IL-4 T-cells and CD8⁺IFNγ⁺ T-cells from plasma of mice following administration of rS1-Fc vaccine via SOFUSA® DoseConnect™.

FIG. 14E is a graph showing box and whisker plots reporting example fold increases in Mean Fluorescence Intensity (MFI) quantification by flow cytometry of CD4⁺IFNγ⁺ T-cells, CD4⁺ IL-4 T-cells and CD8⁺IFNγ⁺ T-cells from plasma of mice following administration of rS1-Fc vaccine via SOFUSA® DoseConnect™ as compared to Mean Fluorescence Intensity (MFI) quantification by flow cytometry of CD4⁺IFNγ⁺ T-cells, CD4⁺ IL-4 T-cells and CD8⁺IFNγ⁺ T-cells from plasma of naïve mice before administration of rS1-Fc vaccine via SOFUSA® DoseConnect™.

FIG. 15A is a Table listing SARS-CoV-2 candidate vaccines in pre-clinical trials (from World Health Organization's draft landscape of COVID-19 candidate vaccines, accessed Nov. 12, 2020).

FIG. 15B is a Table listing SARS-CoV-2 candidate vaccines in clinical trials (from World Health Organization's draft landscape of COVID-19 candidate vaccines, accessed Nov. 12, 2020).

FIG. 16 is a schematic of example microneedle skin penetration depth for each microneedle of an example 4×4 microneedle array (left image) and a graph reporting frequency distribution the example microneedle penetration results for the image shown.

Unless otherwise indicated, the drawings provided herein are intended to illustrate features of embodiments of the disclosure or results of representative experiments illustrating some aspects of the subject matter disclosed herein. These features and/or results are believed to be applicable in a wide variety of systems comprising one or more embodiments of the disclosure. As such, the drawings are not intended to include all additional features known by those of ordinary skill in the art to be required for the practice of the embodiments, nor are they intended to be limiting as to possible uses of the methods disclosed herein.

### DETAILED DESCRIPTION

In the following description, details are set forth by way of example to facilitate discussion of the disclosed subject matter. It should be apparent to a person of ordinary skill in the art, however, that the disclosed embodiment and/or implementations are exemplary and not exhaustive of all possible embodiments and/or implementations.

The present disclosure relates generally to a fluid delivery device, and more specifically relates to a device configured for injection of microliter-scale doses of fluids (herein referred to as a “microdose device”). The present disclosure also relates to methods of applying the microdose device to a patient's skin to deliver a fluidic composition across a dermal barrier of the patient, e.g. to the lymphatic system of the patient. The present disclosure relates more particularly to a microdose device adapted for microliter-scale injections of coronavirus vaccines across a dermal barrier of a patient, e.g. to the lymphatic system of the patient. The present disclosure also relates to methods of administering coronavirus vaccines across a dermal barrier of a patient, e.g. to the lymphatic system of the patient.

As used herein, a “dermal barrier” means a portion of a subject's skin structure. The dermal barrier may include one or more layers of the skin (such as the stratum corneum, epidermis, and/or dermis). In some embodiments, the dermal barrier comprises the stratum corneum of the subject. In some embodiments, the dermal barrier comprises a portion of the epidermis of the subject. In some embodiments, the dermal barrier comprises the entire thickness of epidermis of the subject. In some embodiments, the dermal barrier comprises at least a portion of the dermis of the subject.

As used herein, “lymphatic vasculature” includes any vessel or capillary that carries fluid toward a lymph node or from a lymph node toward a blood vessel. “Proximate to the lymphatic vasculature” means sufficiently close to the lymphatic vasculature for material from a fluidic composition to be taken up into the lymphatic vasculature.

In some embodiments described herein, the microdose device includes an array of microneedles and a fluidic distribution system that can precisely control the flow out of each microneedle. In use, the microneedles are adapted to penetrate the skin to a depth that is distributed between the epidermal and dermal skin layers proximate to the initial lymphatic capillaries. This location of the microneedle can create a predominately unidirectional transfer of a fluid towards the initial lymphatic capillaries. In comparison, for example, conventional subcutaneous injection results in a multidirectional transfer of a fluid that diffuses through Brownian motion in all directions and reduces drug delivery to the initial lymphatic capillaries.

In addition, in some embodiments, a nanopatterned layer that covers the microneedles can further enhance intra-lymphatic drug delivery through increased paracellular and transcellular transport through the epidermal and dermal skin layers.

In various embodiments, the microdose device described herein provides delivery across a dermal barrier of a patient of microliter-scale volumes of pharmaceutical compositions such as coronavirus vaccines and may elicit a superior immune response compared to some existing devices. In some embodiments, higher concentrations of pharmaceutical compositions such as coronavirus vaccines delivered to the lymphatic system of a patient may effectively target dendritic cells below the surface of a patient's skin and elicit a superior immune response compared to other delivery methods.

In addition, the microdose device described herein advantageously provides a cost-effective solution for achieving lymphatic delivery of pharmaceutical compositions such as coronavirus vaccines. In addition to the relative low cost of providing the microdose device itself, the small volume of pharmaceutical compositions such as a coronavirus vaccine to be delivered to patients provides a less costly solution than using larger doses, and therefore a more efficient use of limited supplies of pharmaceutical compositions such as coronavirus vaccines. Advantageously, the microdose device can be used with existing syringes, including but not limited to in some embodiments use with syringes pre-loaded with a pharmaceutical composition such as a coronavirus vaccine. Furthermore, in some embodiments, the microdose device described herein provides lymphatic delivery of doses of pharmaceutical compositions such as a coronavirus vaccine in a shorter period of time than typically achieved with existing devices. Furthermore, the microdose device described herein is simple to use, providing hand-held, manual operability for effective delivery of pharmaceutical compositions such as a coronavirus vaccine to the lymphatic system of a patient, and with less pain than some existing delivery methods, such as intramuscular or intradermal injections.

For the purposes of promoting an understanding of the principles of the present disclosure, reference will now be made to the embodiments and/or implementations illustrated in the drawings, and specific language will be used to describe the same. It will nevertheless be understood that no limitation of the scope of the disclosure is intended. Any alterations and further modifications to the described devices, methods, and any further application of the principles of the present disclosure are fully contemplated as would normally occur to one skilled in the art to which the disclosure relates. In particular, it is fully contemplated that the features, components, and/or steps described with respect to one embodiment and/or implementation may be combined with the features, components, and/or steps described with respect to other embodiments and/or implementations of the present disclosure.

### I. Microdose Device

In some embodiments, a device (herein referred to as “microdose device”) configured for delivering a fluidic composition across a dermal barrier of a patient is described. The microdose device includes a microneedle fluidic block assembly comprising a microneedle array and a fluidic distribution block. The microneedle array comprises a plurality of microneedles disposed on a distal face of a base plate. The microneedles have a fluidic exit channel defined therein, and the microneedles are capable of penetrating the stratum corneum of the skin of a patient and delivering a fluidic composition to a depth below the surface of the skin of the patient. The fluidic distribution block has a distal face coupled to a proximal face of the base plate of the microneedle array. The fluidic distribution block comprises a fluid distribution manifold defined therein and configured to be fluidically connected with the fluidic exit channels of the microneedles and to controllably distribute the fluidic composition to the plurality of microneedles through the fluidic exit channels. The microdose device also has a syringe connection assembly having a fluidic path defined therein. The syringe connection assembly comprises a distal end coupled to a proximal face of the fluidic distribution block, wherein the fluidic path of the syringe connection assembly is fluidically connected to the fluid distribution manifold. The syringe connection assembly also has a proximal end configured to be coupled to a syringe barrel having a bore defined therein, the fluidic path of the syringe connection assembly configured to be fluidically connected to the bore of the syringe barrel.

Certain example embodiments of the microdose device are illustrated in the drawings and described herein.

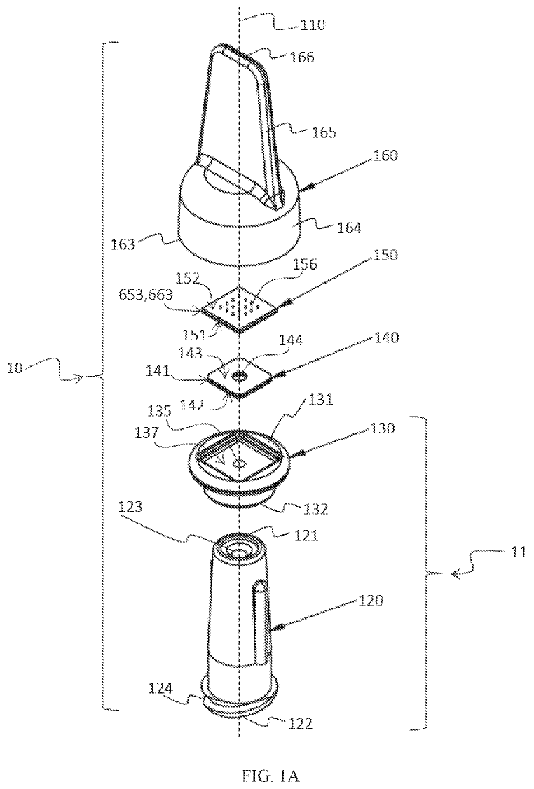

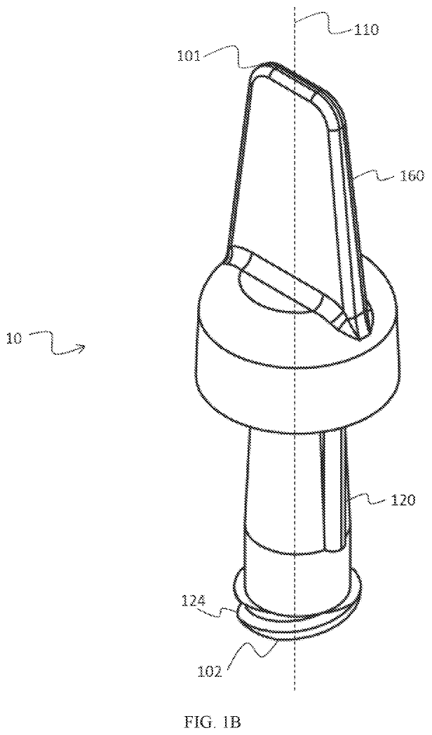

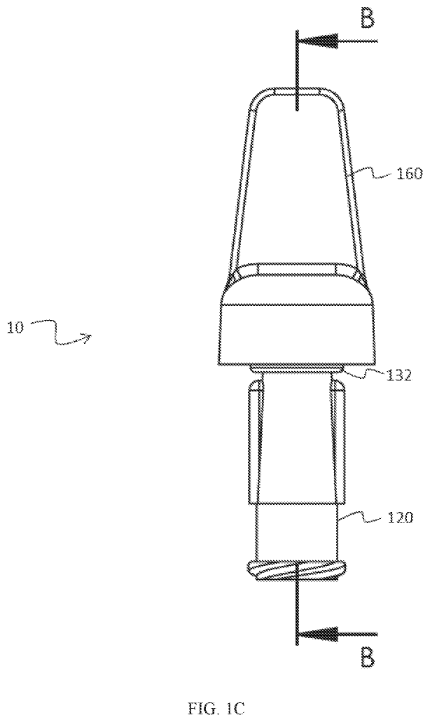

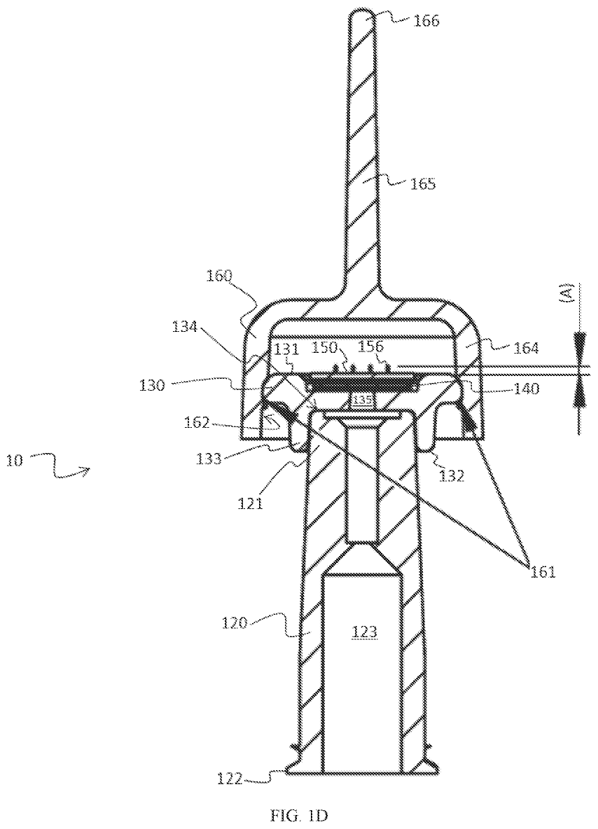

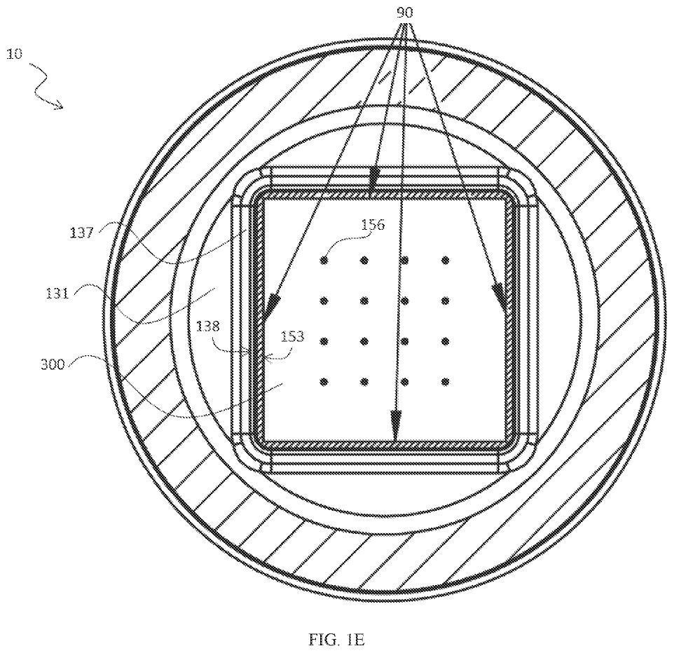

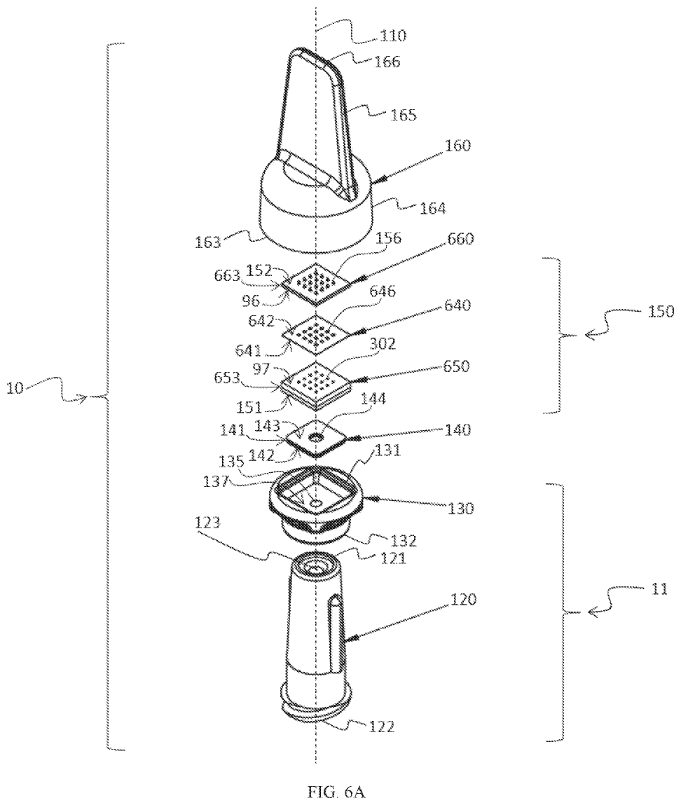

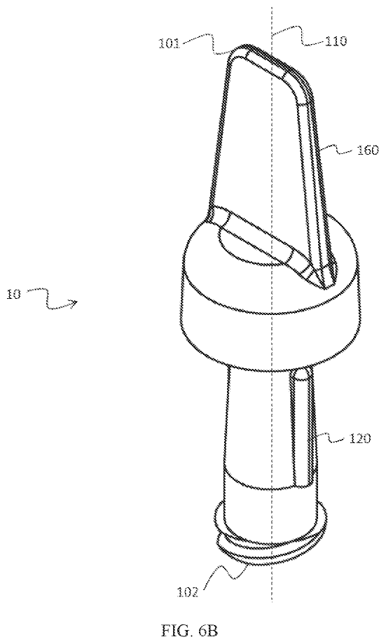

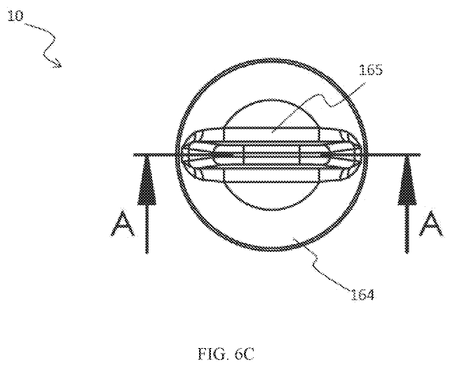

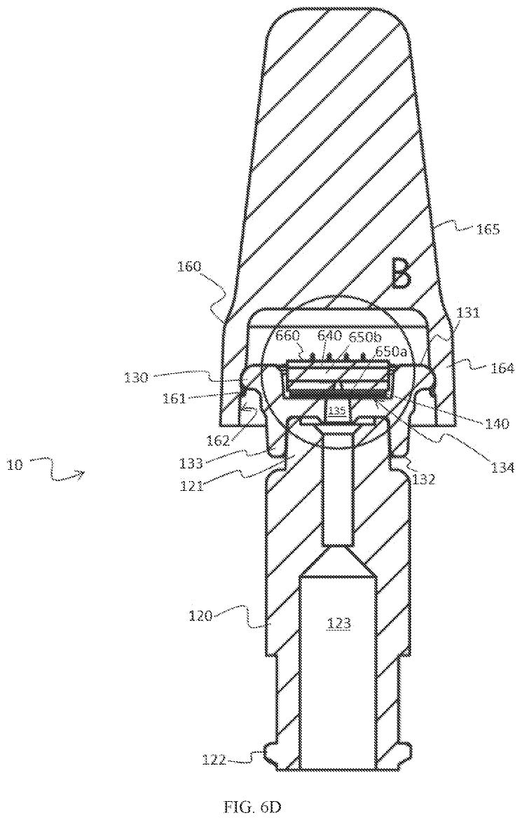

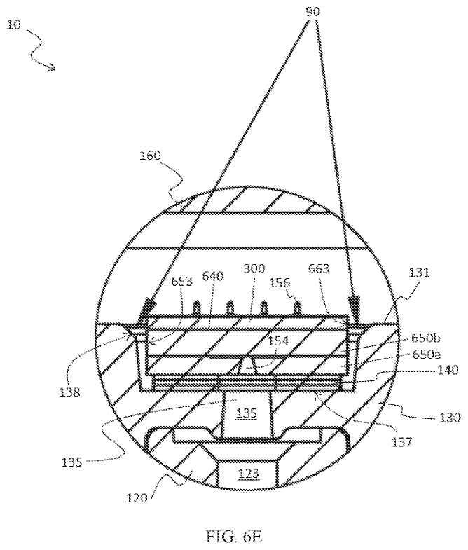

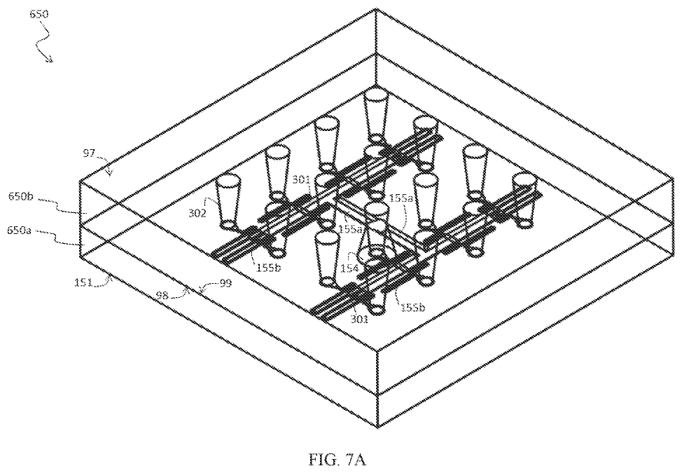

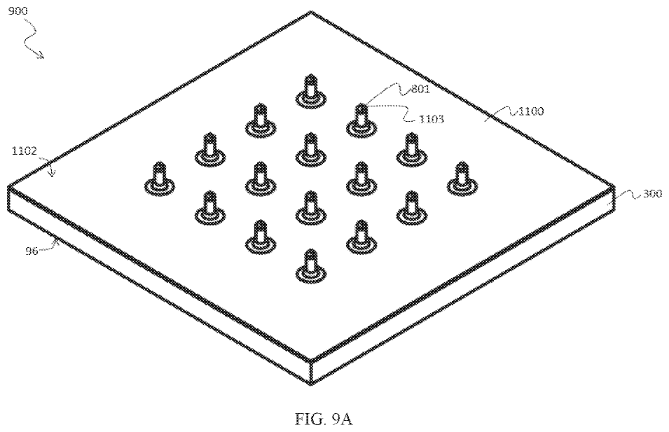

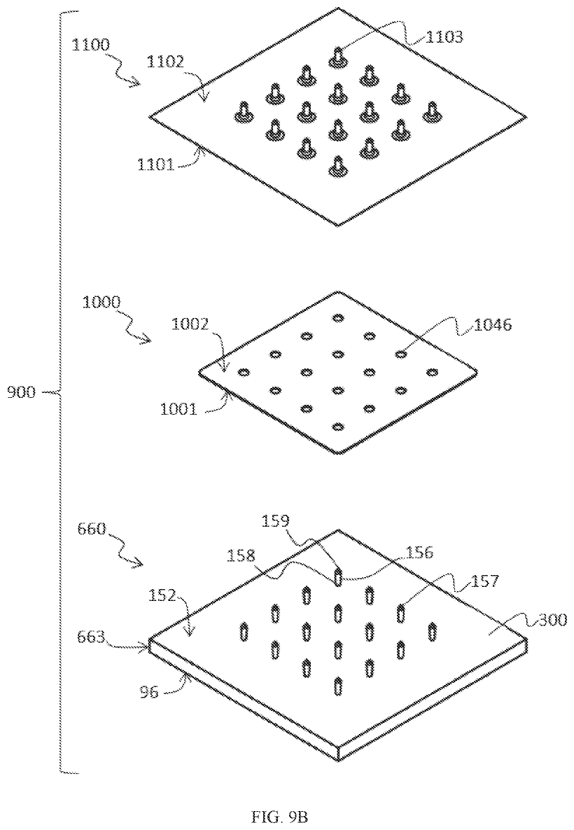

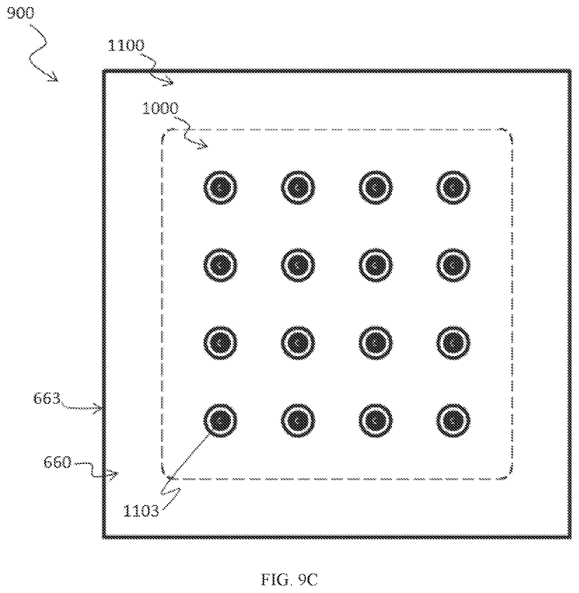

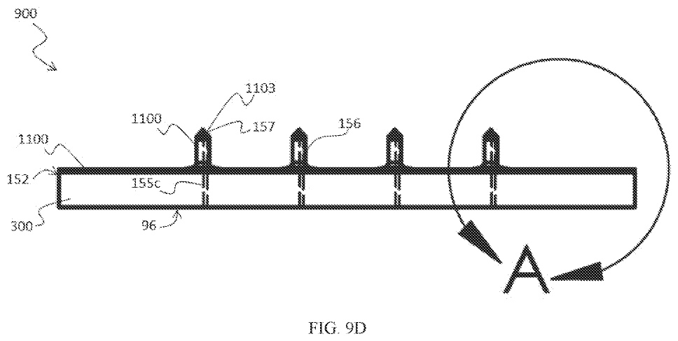

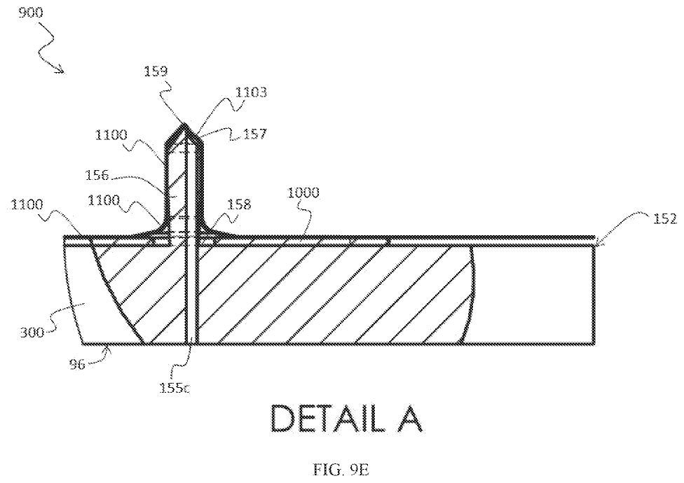

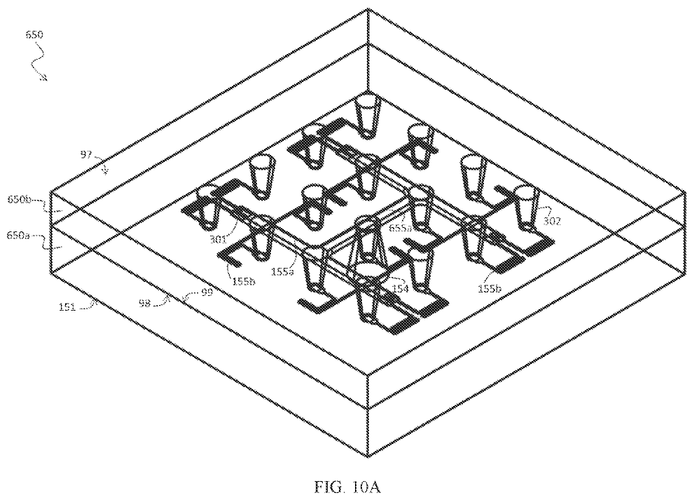

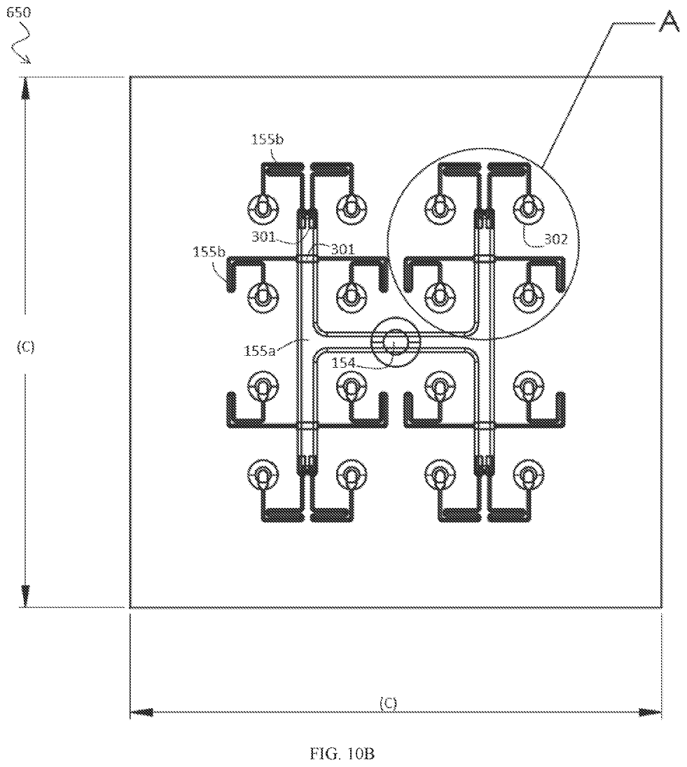

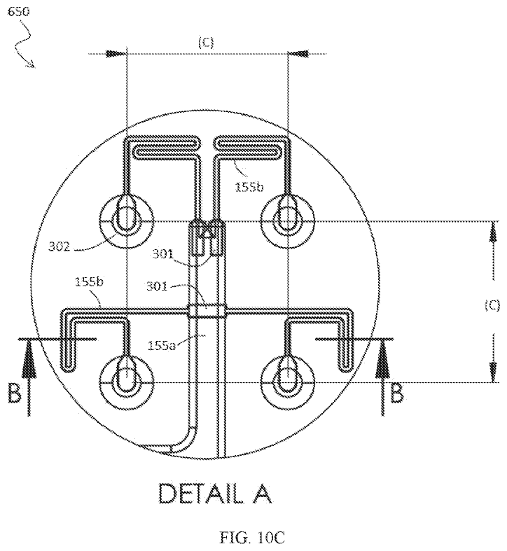

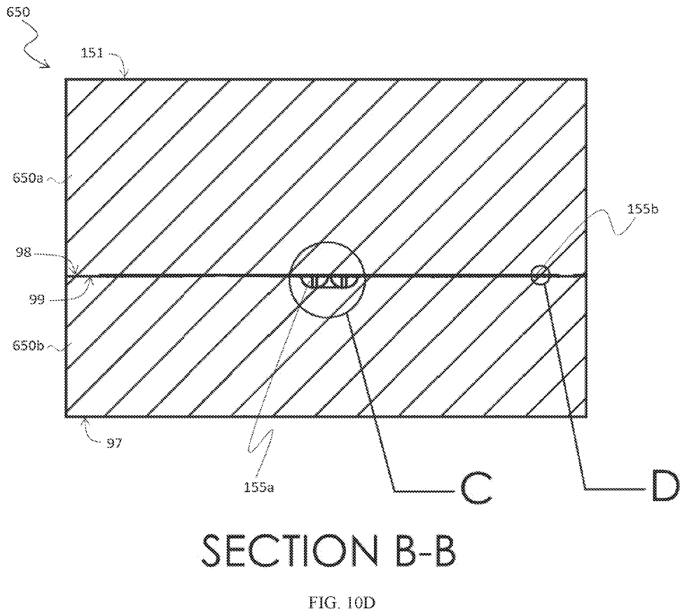

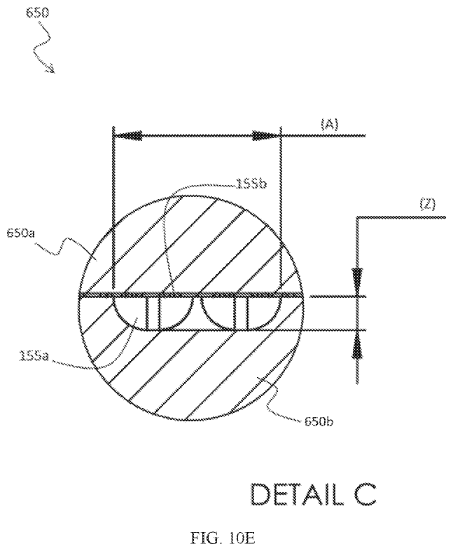

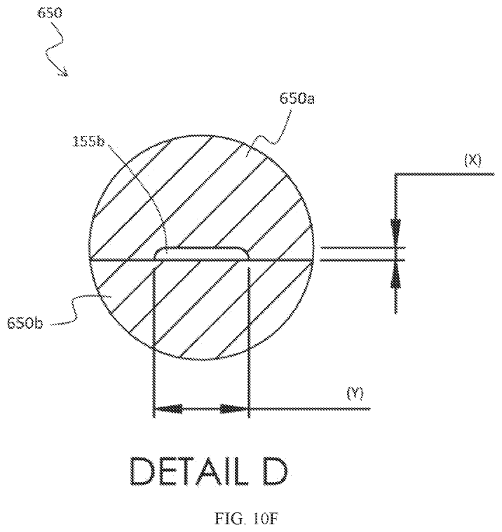

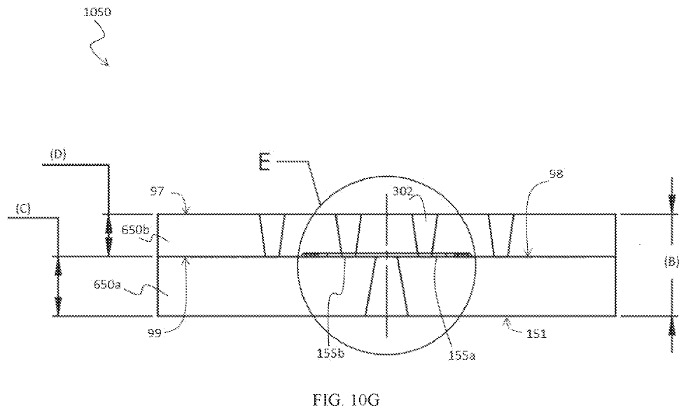

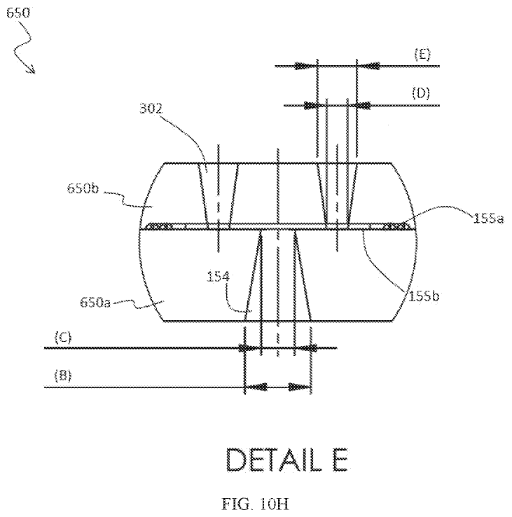

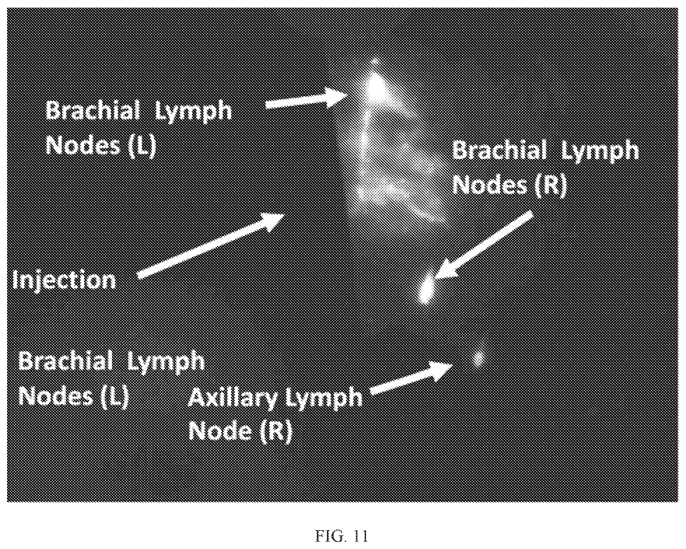

For example, FIG. 1A to FIG. 5E of the present disclosure illustrate a first example microdose device 10 and components thereof. FIG. 6A to FIG. 8E of the present disclosure illustrate a second example microdose device 10 and components thereof. FIG. 9A to FIG. 10H of the present disclosure illustrate additional example embodiments of the example microdose device 10 and components thereof.

With reference to FIG. 1A, FIG. 1B, FIG. 6A, and FIG. 6B, dashed line 110 shows the longitudinal axis of the example microdose devices 10 and components thereof described herein and illustrated in the drawings. Accordingly, it is to be understood that schematic illustrations in the drawings herein having an axial plan view are viewed along the longitudinal axis. It is also to be understood that schematic illustrations in the drawings herein described herein having a side view are viewed at an angle substantially perpendicular to the longitudinal axis, and the term “lateral” refers to an orientation substantially perpendicular to the longitudinal axis of the microdose device 10 or a component thereof. The term “top-down” as used herein with regard to a view of a drawing is understood to refer to an axial view from the distal end 101 of the microdose device 10, or a component thereof, toward the proximal end 102 of the microdose device 10 or a component thereof. The term “bottom-up” as used herein with regard to a view of a drawing is understood to refer to an axial view from the proximal end 102 of the microdose device 10, or a component thereof, toward the distal end 101 of the microdose device or a component thereof. It is to be understood that as described herein, when in use, a fluidic composition flows in a generally proximal to distal direction when being delivered to a patient from the microdose device.

Turning to FIG. 1A, a perspective exploded view schematic is shown of an example first set of components adapted to be combined to form an example microdose device 10, as shown in FIG. 1B and described herein.

The components of the example microdose device 10 shown in FIG. 1A include a syringe connection assembly 11 comprising a tubing connector 120 and a plenum 130. The components of the example microdose device 10 shown in FIG. 1 also include a first gasket 140, a microneedle fluidic block assembly 150, and a protective cap 160.

As would be understood from the example drawings in FIG. 1A and FIG. 1B, in some embodiments, a microdose device of the present disclosure, such as the example microdose device 10 shown in FIG. 1B, may be assembled from the example components shown in FIG. 1A as described herein.

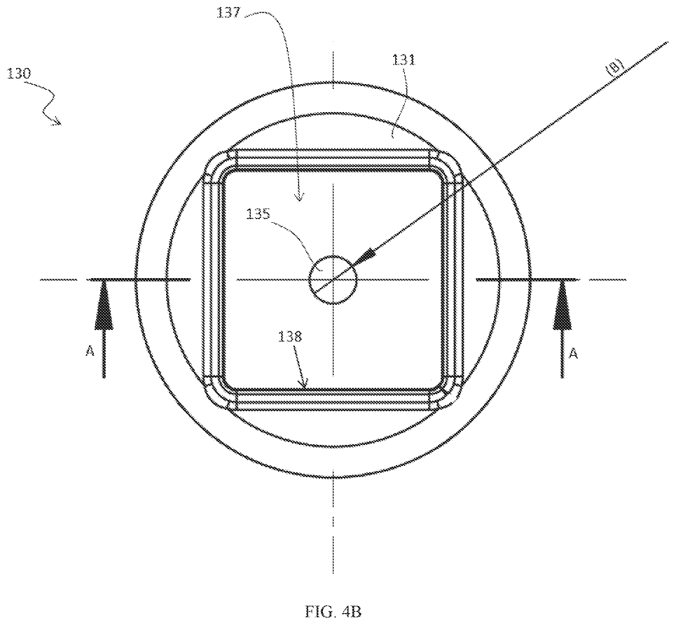

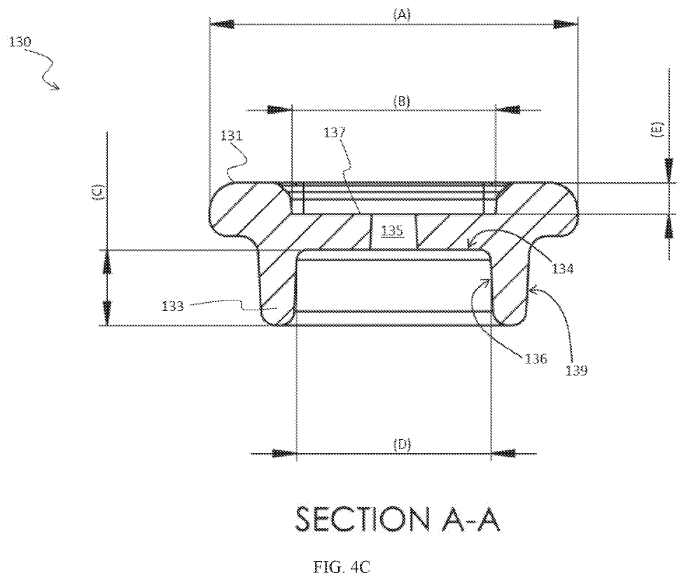

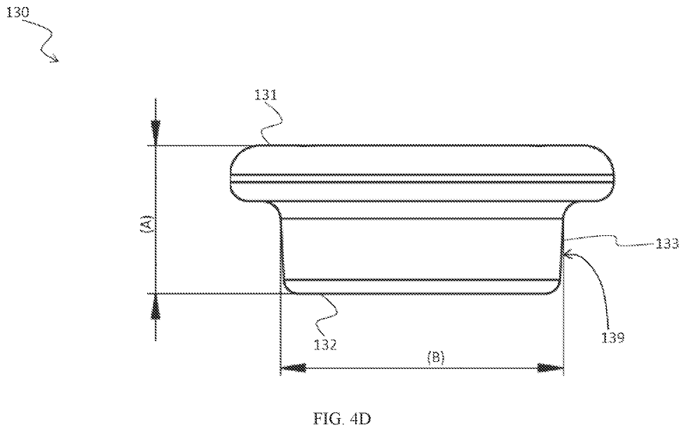

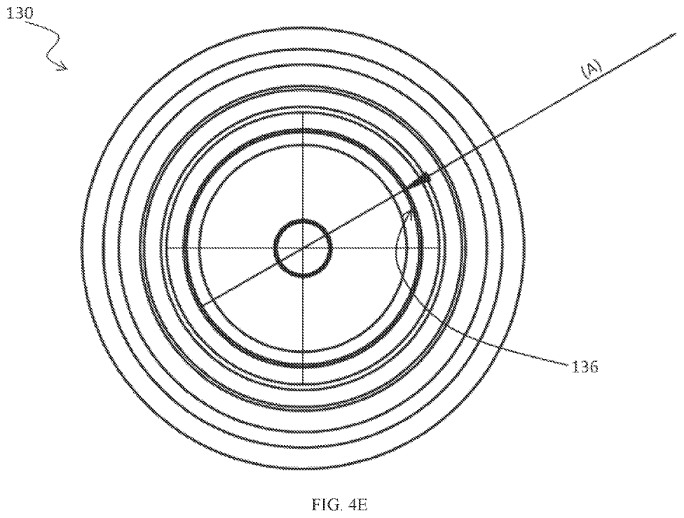

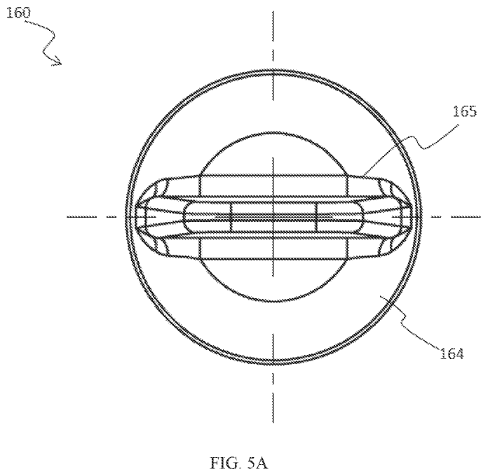

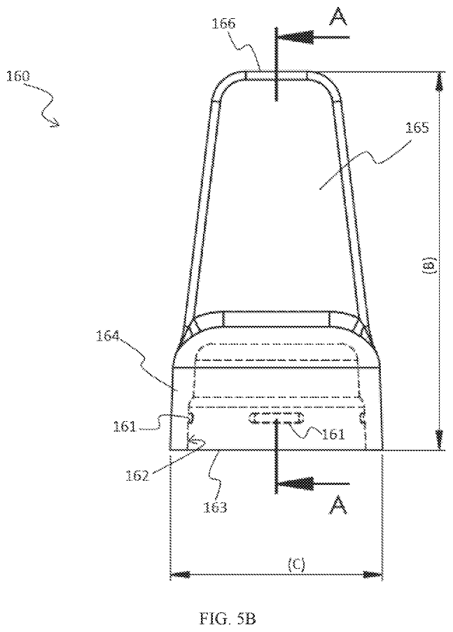

In some embodiments, the syringe connection assembly may be provided as a single component, for example formed in a single molded piece. In other embodiments, the syringe connection assembly may comprise one or more sub-components configured to be fluidically connected to the bore of a syringe barrel to the fluid distribution manifold defined within the microneedle fluidic block assembly. For example, as shown in the example microdose devices described herein, in some embodiments, the syringe connection assembly may comprise a plenum coupled to and fluidically connected to the microneedle fluidic block assembly, and may further comprise a tubing connector coupled to and fluidically connected to the plenum and configured to be coupled to and fluidically connected to a syringe barrel. With reference to the first example microdose device 10, as shown in FIG. 1A and FIG. 1B, the example syringe connection assembly 11 comprises an example tubing connector 120 and a plenum 130. A distal portion 121 of the tubing connector 120 is adapted to be coupled to a proximal face 132 of the plenum 130. The distal portion 121 of the tubing connector 120 may be adapted to be slidably coupled to the proximal face 132 of the plenum 130. As shown in further detail in FIG. 1D, the proximal portion 121 of the tubing connector 120 may be adapted to be slidably coupled within a proximal well 133 of the plenum 130. The distal portion 121 of the tubing connector 120 may contact a proximal seat 134 of the plenum 130, at the distal end of the proximal well 133. The tubing connector 120 defines a tube 123 therein adapted to allow a fluid to flow therethrough. The distal portion 121 of the tubing connector 120 and the proximal face 132 of the plenum 130 are adapted to be coupled together to allow a fluid to flow from within the tube 123 of the tubing connector 120 to an orifice 135 disposed within the plenum 130. It is to be understood that in the example microdose device 10 of FIG. 1A, the tube 123 of the tubing connector 120 and the orifice 135 of the plenum 130 are adapted to be fluidically connected together to form the fluidic path of the syringe connection assembly 11. The proximal seat 134 of the plenum 130 and the distal portion 121 of the tubing connector 120 may be coupled together such that the fluidic connection between the tube 123 and the orifice 135 is sealed such that the coupling prevents leakage of a fluid passing therethrough. In some embodiments, for example as in the example shown in FIG. 1A-1E, in which the distal portion 121 of the tubing connector 120 is adapted to be slidably coupled to the proximal face 132 of the plenum 130, an inner wall 136 (see e.g. FIG. 4C) of the proximal well 133 of the plenum 130 and the distal portion 121 of the tubing connector 120 may be coupled together with a friction fit such that the fluidic connection between the tube 123 and the orifice 135 prevents leakage of a fluid passing therethrough.

In some embodiments, the distal portion 121 of the tubing connector 120 and the proximal face 132 of the plenum 130 may be adhered together, for example using an adhesive such as Loctite 3979, or other suitable adhesive identifiable by skilled persons. The adhering of the distal portion 121 of the tubing connector 120 and the proximal face 132 of the plenum 130 may provide a sealed fluidic connection such that the adhering of the distal portion 121 of the tubing connector 120 and the proximal face 132 of the plenum 130 prevents leakage of a fluid passing therethrough.

In some embodiments, the distal portion 121 of the tubing connector 120 may be adapted to be coupled to the proximal face 132 of the plenum 130 by compatible screw threads. In such embodiments, the distal portion 121 of the tubing connector 120 may have a screw thread disposed thereon that is compatible with a screw thread disposed on the proximal face 132 of the plenum 130. Accordingly, in such embodiments, for example, the inner wall 136 (see e.g. FIG. 4C) of the proximal well 133 of the plenum 130 and the distal portion 121 of the tubing connector 120 may be adapted to form a sealed, coupling between the tube 123 and the orifice 135 that prevents leakage of a fluid passing therethrough when coupled together via the respective screw threads disposed thereon.

In some embodiments, the plenum 130 and the tubing connector 120 may be coupled together by an adhesive bond, a weld joint (e.g., spin welding, ultrasonic welding, laser welding, or heat staking), and the like. In some embodiments, the plenum 130 and the tubing connector 120 may be coupled together using any connection technique that enables the formation of the syringe connection assembly 11.

In some embodiments, the plenum 130 and the tubing connector 120 may be formed from any suitable material, e.g. a plastic material or other polymer, for example such as a cyclic olefin copolymer among others.

In some embodiments, the tubing connector 120 may be a commercially available component such as a female luer adapter available from Qosina Corp. (Ronkonkoma, New York; Part. No. 11203).

In some embodiments, when the tubing connector 120 is coupled to the plenum 130 to form a syringe connection assembly 11, the axial length from the proximal end 122 of the tubing connector 120 to a distal face 131 of the plenum 130 may be about 17 mm.

In some embodiments, the microdose device 10 may include a first gasket 140 disposed between and coupled to the syringe connection assembly 11 and the microneedle fluidic block assembly 150. For example, in some embodiments, such as illustrated in FIG. 1A, the first gasket 140 is adapted to be coupled to the distal face 131 of the plenum 130. A proximal face 142 of the first gasket 140 may be coupled to the plenum 130 within a distal seat 137 disposed within the distal face 131 of the plenum 130. The first gasket 140 may be sized such that the lateral edges 141 of the first gasket 140 are adapted to be coupled to an inner wall 138 (see e.g. FIG. 4A) surrounding the distal seat 137 disposed in the distal face 131 of the plenum 130. The proximal face 142 of the first gasket 140 may be adapted to adhere to the distal face 131 of the plenum 130. The first gasket 140 may be a pressure-sensitive adhesive (PSA) gasket. The first gasket 140 may have an adhesive disposed on the proximal face 142 of the first gasket 140 and/or the distal face 143 of the first gasket 140. The first gasket 140 has a hole 144 adapted to allow a fluid to flow through the first gasket 140 from the proximal face 142 of the first gasket 140 to the distal face 143 of the first gasket 140. When the first gasket 140 is coupled to the plenum 130, the hole 144 of the first gasket 140 may be fluidically connected to the orifice 135 of the plenum 130. In some embodiments, the device may not include a first gasket 140 disposed between and coupled to the syringe connection assembly 11 and the microneedle fluidic block assembly 150. In some embodiments, a distal portion of the syringe connection assembly 11 e.g. the distal face of the plenum 130 may be coupled to the microneedle fluidic block assembly 150, for example by over-molding a distal portion of the syringe connection assembly 11 e.g. the distal face of the plenum 130 to around a portion of the microneedle fluidic block assembly 150. In some embodiments, the over-molding of the distal portion of the syringe connection assembly 11 e.g. the distal face of the plenum 130 may provide a leak-proof provide a leak-proof fluidic connection between the syringe connection assembly 11 and the microneedle fluidic block assembly 150.

In some embodiments, the lateral diameter of the distal face 131 of the plenum 130 may be about 10.33 mm (see e.g., “(A)” indicated in FIG. 4C). In some embodiments, the lateral diameter of the distal seat 137 of the plenum 130 may be about 5.71 mm (see e.g., “(B)” indicated in FIG. 4C). In some embodiments, the axial depth of the proximal well 133 of the plenum 130 may be about 2.12 mm (see e.g., “(C)” indicated in FIG. 4C). In some embodiments, the lateral inner diameter within the inner walls 136 of the proximal well 133 of the plenum 130 may be about 5.45 mm (see e.g., “(D)” indicated in FIG. 4C and “(A)” indicated in FIG. 4E). In some embodiments, the axial depth of the distal seat 137 of the plenum 130 may be about 0.88 mm (see e.g., “(E)” indicated in FIG. 4C).

In some embodiments, the lateral diameter of the orifice 135 of the plenum 130 may be about 1.23 mm. (e.g., see “(B)” indicated in FIG. 4B). In some embodiments, the diameter of the hole 144 of the first gasket 140 may be about 1.23 mm.

In some embodiments, the axial length between the proximal face 132 of the plenum 130 and the distal face 131 of the plenum 130 may be about 4.00 mm (see e.g., “(A)” indicated in FIG. 4D). In some embodiments, the lateral outer diameter of an outer wall 139 of the proximal well 133 of the plenum 130 may be about 7.61 mm (see e.g., “(B)” indicated in FIG. 4D).

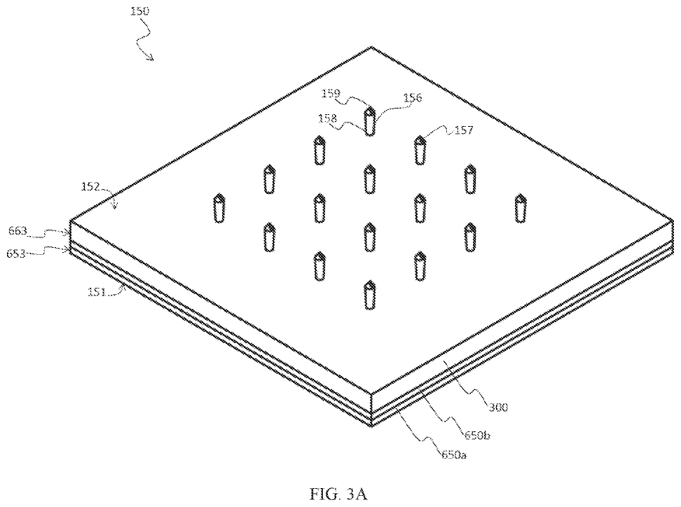

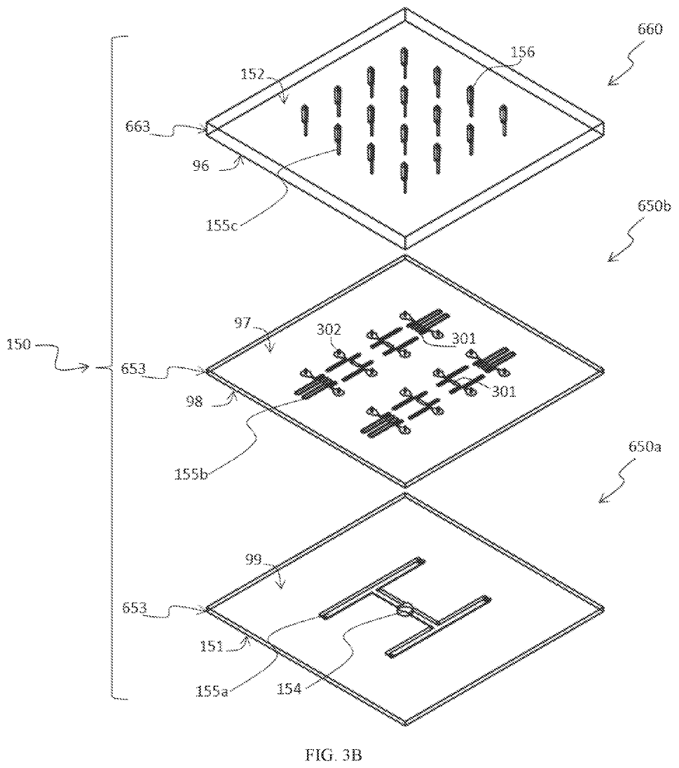

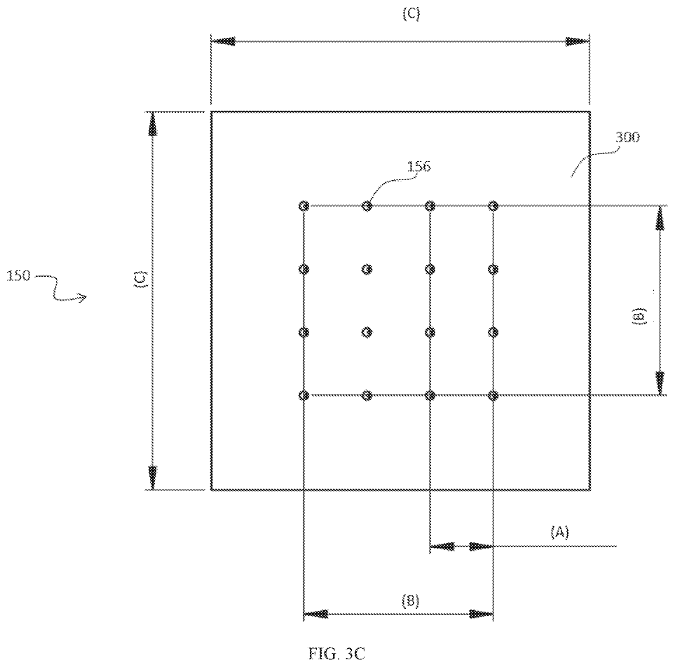

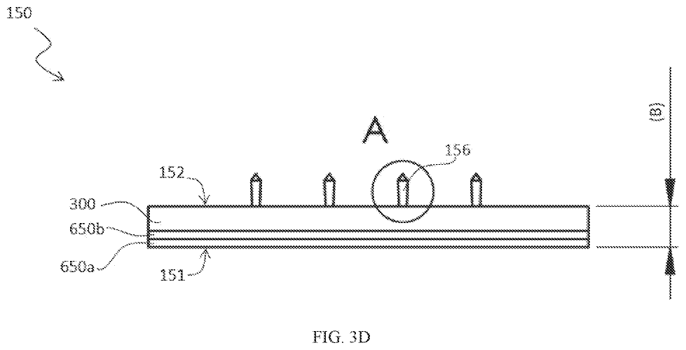

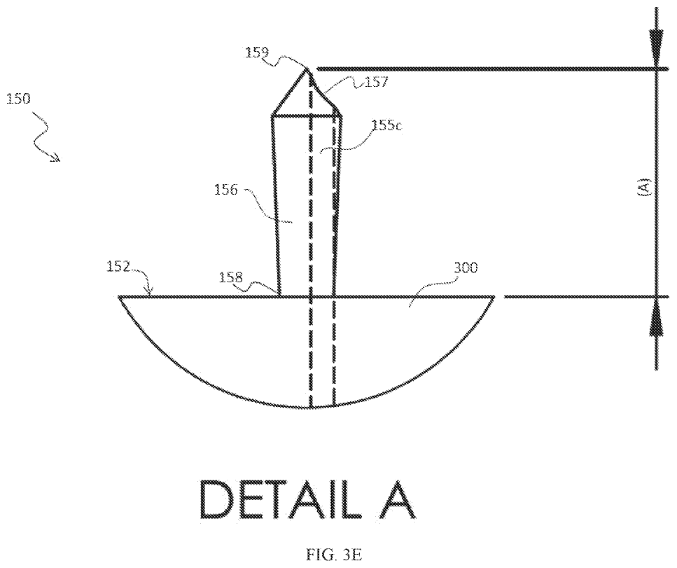

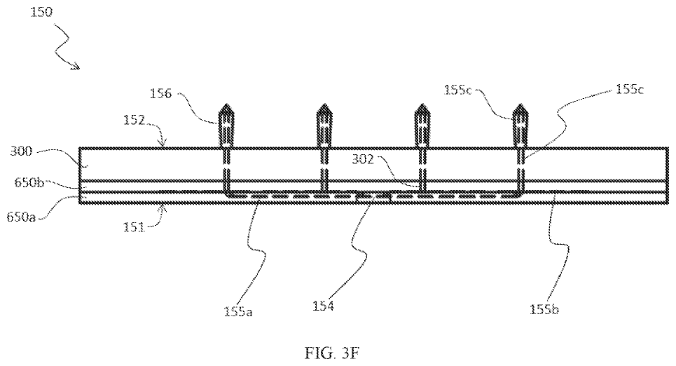

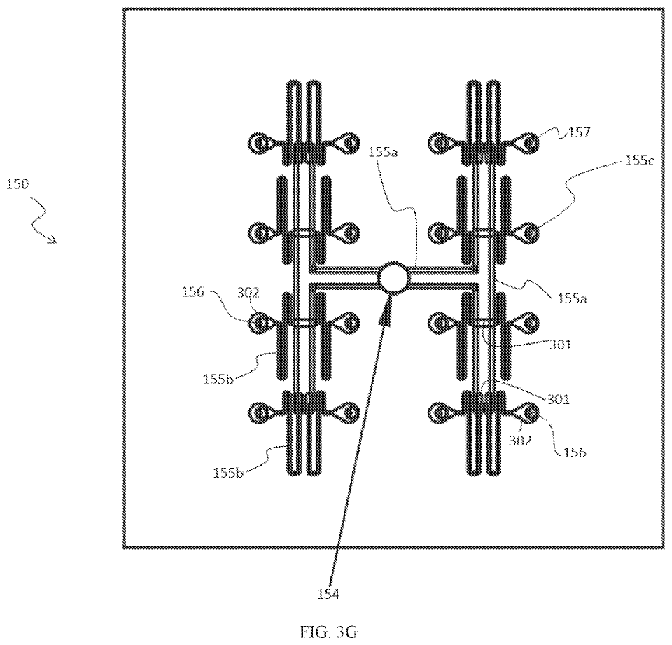

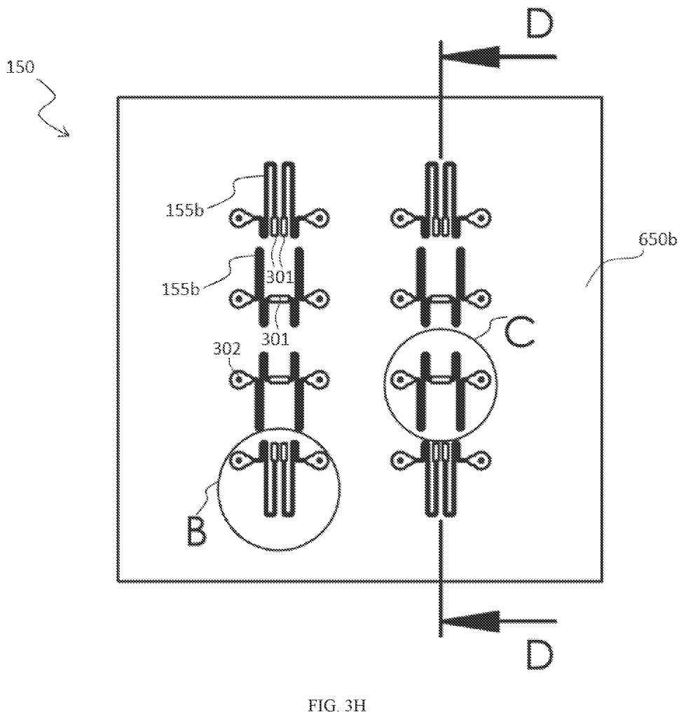

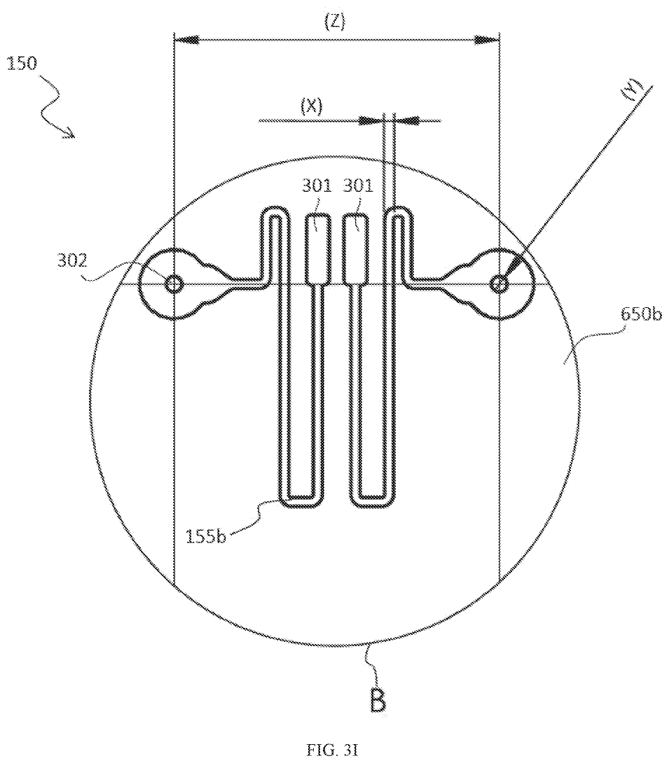

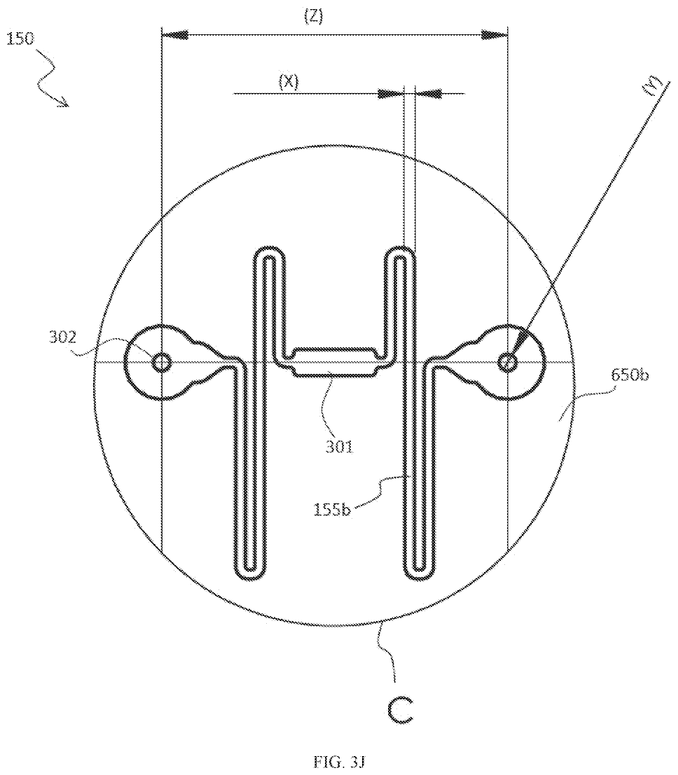

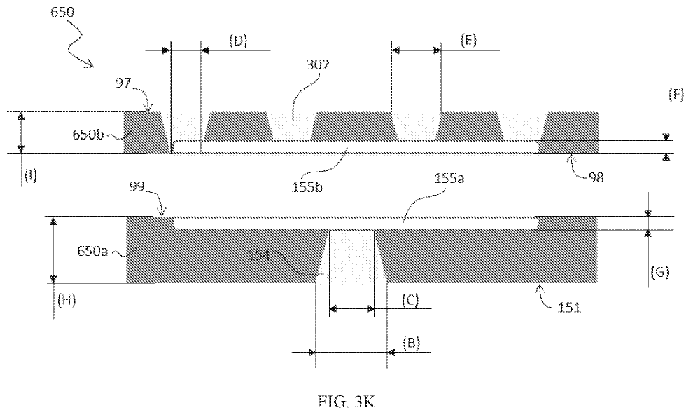

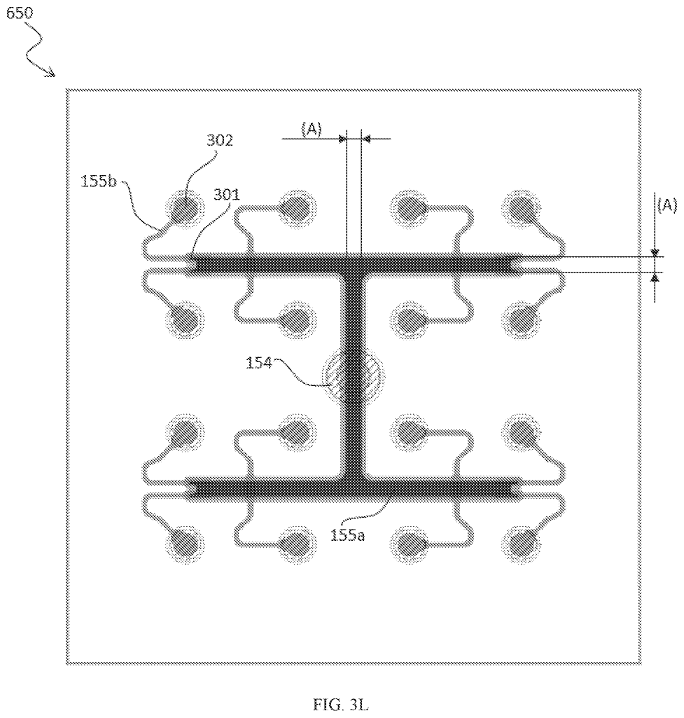

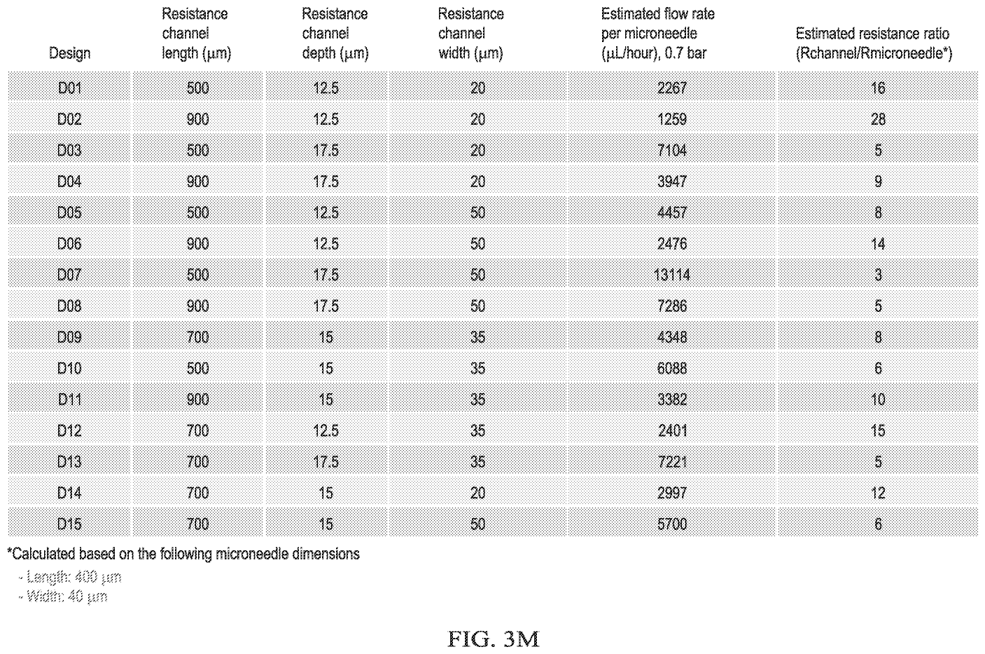

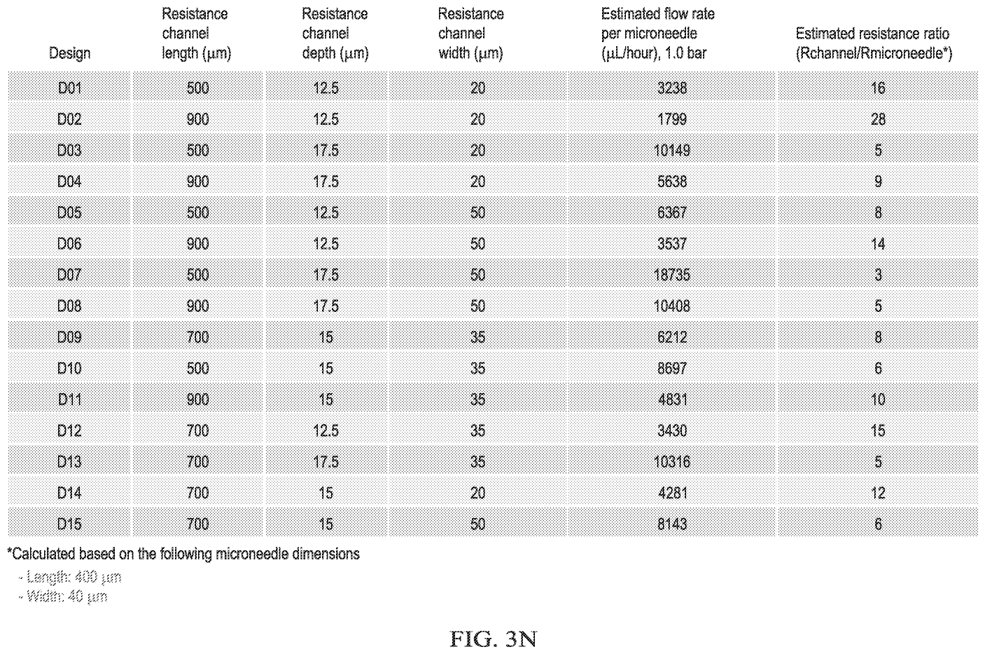

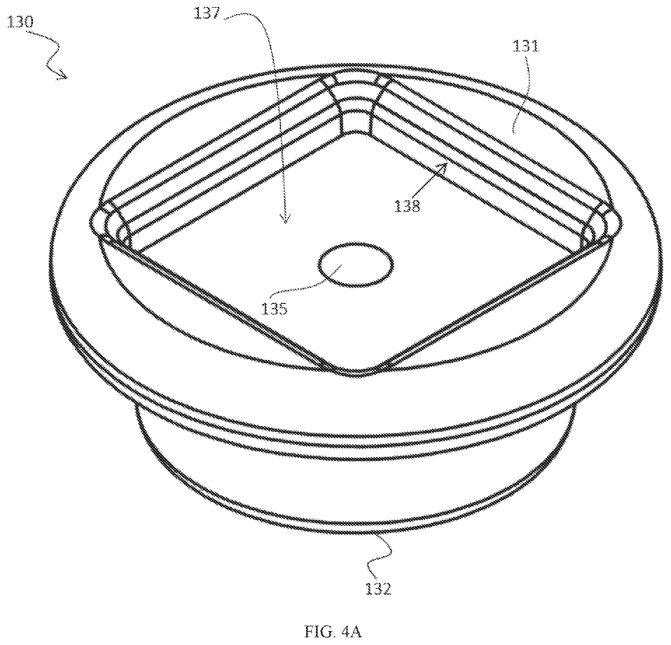

Turning now to FIG. 3A to FIG. 3J, the microneedle fluidic block assembly 150 of the first example microdose device 10 includes a microneedle array 660 comprising a plurality of microneedles 156 disposed on a distal face 152 of a base plate 300, the base plate 300 also having a proximal face 96 (see, e.g., FIG. 1A and FIG. 3A-FIG. 3J). Each of the microneedles 156 has a base 158 coupled to the distal face 152 of the base plate 300 and extending away distally from the base plate 300 to a distal end comprising a tip 159. The tip 159 may have a piercing or needle-like shape, such as a conical or pyramidal shape or a cylindrical shape transitioning to a conical or pyramidal shape.

The tip 159 of each microneedle 156 is disposed furthest away from the base plate 300 and may define the smallest dimension (e.g., diameter or cross-sectional width) of each microneedle 156. Additionally, each microneedle 156 may generally define any suitable axial length between its base 158 and its tip 159 that is sufficient to allow the microneedles 156 to penetrate the stratum corneum of a patient. In some embodiments, it may be desirable to limit the length of the microneedles 156 such that they do not penetrate through the inner surface of the epidermis and into the dermis, which may advantageously help minimize pain for the patient receiving the fluid.

In some embodiments, each microneedle 156 may, have an axial length of from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some embodiments, each microneedle 156 may have an axial length of less than about 1000 micrometers (μm) such as less than about 800 μm, or less than about 750 μm, or less than about 500 μm (e.g., a length ranging from about 200 μm to about 400 μm), or any other sub-ranges therebetween. In one example, the microneedles 156 may have an axial length from the base 158 to the tip 159 of about 400 μm (e.g., indicated by “(A)” in FIG. 3E).

The length of the microneedles 156 may be varied depending on the intended location at which the microdose device 10 is to be used on a patient. For example, the length of the microneedles 156 for a microdose device 10 to be used on a patient's leg may differ substantially from the length of the microneedles 156 for a microdose device 10 to be used on a patient's arm Each microneedle 156 may generally define any suitable aspect ratio (i.e., the axial length over a cross-sectional lateral width dimension of each microneedle 156). The aspect ratio may be greater than 2, such as greater than 3 or greater than 4. In instances in which the cross-sectional width dimension (e.g., diameter) varies over the length of each microneedle 156, the aspect ratio may be determined based on the average cross-sectional width dimension.

As used herein, an “aspect ratio” means the ratio of the axial length of a microneedle to the cross-sectional lateral dimension perpendicular to the length (e.g., width or diameter) of the microneedle. In instances in which the cross-sectional dimension (e.g., diameter of the protrusion having a conical shape) varies over the length, the aspect ratio is determined based on the average cross-sectional lateral dimension unless otherwise indicated.

In some embodiments, the plurality of microneedles 156 are adapted for penetrating the patient's skin, and delivering a fluid such as a liquid pharmaceutical composition to the patient's lymphatic system.

Example microneedle arrays include those described in WO2012/020332, WO20111070457, WO 2011/135532, US2011/0270221, US2013/0165861, US 2019/90143090, and U.S. provisional patent application Nos. 61/996,148 and 62/942,971, each of which is incorporated herein by reference in its entirety.

Generally, the microneedle fluidic block assembly 150 may have any suitable configuration known in the art for delivering a fluid through the patient's skin to the patient's lymphatic system. The fluidic distribution block 650, base plate 300 and microneedles 156 may generally be constructed from a rigid, semi-rigid or flexible sheet of material, such as a metal material, a ceramic material, a polymer (e.g., plastic) material and/or any other suitable material. For example, the fluidic distribution block 650, base plate 300 and microneedles 156 may be formed from silicon by way of reactive-ion etching, or in any other suitable manner.

In some embodiments, such as in FIG. 1A, the proximal face 151 of the microneedle fluidic block assembly 150 is adapted to be coupled to the distal face 143 of the first gasket 140. The proximal face 151 of the microneedle fluidic block assembly 150 may be adapted to be coupled to the distal face 143 of the first gasket 140, for example by adhering the proximal face 151 of the microneedle fluidic block assembly 150 to the distal face 143 of the first gasket 140.

In some embodiments, the plenum 130 is adapted to be slidably coupled to the microneedle fluidic block assembly 150 and configured to hold the microneedle fluidic block assembly 150. As shown for example in FIG. 1E, the base plate 300 of the microneedle fluidic block assembly 150 may be sized such that the lateral edges 663 of the base plate 300 are adapted to be coupled to the inner wall 138 of the distal seat 137 disposed in the distal face 131 of the plenum 130. The lateral edges 663 of the base plate 300 may be adhered to the inner wall 138 of the distal seat 137 disposed in the distal face 131 of the plenum 130, for example using an adhesive such as Loctite 3979, or other suitable adhesive identifiable by skilled persons. An adhesive may be applied, e.g. in a continuous bead, between the lateral edges 663 of the base plate 300 and the inner wall 138 of the distal seat 137 disposed in the distal face 131 of the plenum 130 (e.g., see adhesive 90 in FIG. 1E).

In some embodiments, the plenum 130 and the microneedle fluidic block assembly 150 may be coupled together for example, and without limitation, via an adhesive bond, a weld joint (e.g., spin welding, ultrasonic welding, laser welding, or heat staking), and the like.

In some embodiments, the microneedle fluidic block assembly 150 includes a fluidic distribution block 650 that includes a fluid distribution manifold that extends through the fluidic distribution block 650. The fluid distribution manifold is configured to provide a substantially uniform supply, e.g. a substantially equal flow rate, of a fluidic composition to the exit channels 155c in each microneedle 156. A distal face 97 of the fluidic distribution block 650 may be bonded to the proximal face 96 of the base plate 300, e.g. by an adhesive. The fluid distribution manifold may be configured for supplying a fluidic composition to the fluidic exit channels 155c in one or more microneedles 156, for example, as depicted in FIG. 3B.

In some embodiments, the fluid distribution manifold includes a proximal entrance 154 forming an opening in the proximal face 151 of the fluidic distribution block 650 (see, e.g., FIG. 3B). In some embodiments, the proximal entrance 154 is adapted to allow a fluid to flow from the hole 144 of the first gasket 140, when present, into supply channels 155a and resistance channels 155b (see below) of the fluid distribution manifold disposed within the fluidic distribution block 650.

In some embodiments, the first gasket 140 may be absent, such that the proximal face 151 of the fluidic distribution block 650 may be coupled to the distal end of the syringe connection assembly, e.g. the distal face 131 of the plenum 130, and wherein the proximal entrance 154 of the fluidic distribution block 650 is fluidically connected with the fluidic path of the syringe connection assembly 11. In some embodiments, for example, the proximal face 151 of the fluidic distribution block 650 and the distal face 131 of the plenum 130 may be adhered together, for example using an adhesive such as Loctite 3979, or other suitable adhesive. The adhering of the proximal face 151 of the fluidic distribution block 650 and the distal face 131 of the plenum 130 may further provide a sealed coupling between the proximal face 151 of the fluidic distribution block 650 and the distal face 131 of the plenum 130 adapted to prevent leakage of a fluid when the fluid passes from the fluidic path of the syringe connection assembly 11 into the proximal entrance 154 of the fluidic distribution block 650.

The fluid distribution manifold is adapted to receive a fluid through the proximal entrance 154 of the microneedle fluidic block 150, and distribute the fluid to the fluidic exit channels 155c in one or more microneedles 156 (see e.g. FIG. 3A-FIG. 3F).

In some embodiments, for example as shown in FIG. 1D, when the microneedle fluidic block assembly 150 is disposed within the distal seat 137 in the distal face 131 of the plenum 130, an axial distance (e.g., indicated by “(A)” in FIG. 1D) between the tip 159 of the microneedles 156 and the distal face 131 of the plenum 130 may be about 0.45 mm.

In some embodiments, the fluidic distribution block 650 may be formed by bonding a proximal layer 650a including the proximal entrance 154 formed through the proximal layer 650, to a distal layer 650b including the outlet apertures 302 formed therethrough. The supply channels 155a and/or resistance channels 155b may be formed on the distal face 99 of the proximal layer 650a and/or the proximal face 98 of the distal layer 650b.

In some embodiments, the proximal layer 650a and the distal layer 650b of the fluidic distribution block 650 may comprise a glass material. In some embodiments, the proximal layer 650a and the distal layer 650b of the fluidic distribution block 650 may comprise silicon. The proximal layer 650a and the distal layer 650b may be fabricated from different materials of any combination that enables the fluidic distribution block 650 to function as described herein. In some embodiments, the proximal layer 650a may comprise glass and the distal layer 650b may comprise silicon.

The entrance 154 may be formed in the proximal layer 650a by drilling, cutting, etching, and/or powder blasting, or any other manufacturing technique for forming a channel or aperture through the proximal layer 650a. In some embodiments, the supply channels 155a and the resistance channels 155b are formed in the distal face 99 of the proximal layer 650a and/or the proximal face 98 of the distal layer 650b using an etching technique. For example, in some embodiments, wet etching, or hydrofluoric acid etching, is used to form the supply channels 155a and the resistance channels 155b. In another suitable embodiment, Deep Reactive Ion Etching (DRIE or plasma etching) may be used to create deep, high density, and high aspect ratio supply channels 155a and resistance channels 155b in distal face 99 of the proximal layer 650a and/or the proximal face 98 of the distal layer 650b. Alternatively, the supply channels 155a and resistance channels 155b can be formed in distal face 99 of the proximal layer 650a and/or the proximal face 98 of the distal layer 650b using any fabrication process that enables the fluidic distribution block 650 to function as described herein. The outlet apertures 302 may be formed through the distal layer 650b by drilling, cutting, etching, and/or powder blasting, or any other manufacturing technique for forming a channel or aperture through the distal layer 650b. Suitable commercially available etching and lithography processes that may be used in producing the channels, microneedles, and so on, of the microdose device described herein are available, for example from Micronit Micro Technologies BV, Enschende, Netherlands.

In some embodiments, the proximal layer 650a and the distal layer 650b may be bonded together in face-to-face contact to seal the edges of the supply channels 155a and the resistance channels 155b of the fluid distribution manifold to provide a leak-proof fluidic connection between the supply channels 155a and the resistance channels 155b of the proximal layer 650a and the distal layer 650b. In some embodiments, direct bonding, or direct aligned bonding, may be used by creating a pre-bond between the proximal layer 650a and the distal layer 650b. The pre-bond can include applying a bonding agent to the distal face 99 of the proximal layer 650a and/or the proximal face 98 of the distal layer 650b before bringing the proximal layer 650a and the distal layer 650b into direct contact. The proximal layer 650a and the distal layer 650b may be aligned and brought into face-to-face contact and annealed at an elevated temperature. In some embodiments, anodic bonding may be used to bond the proximal layer 650a and the distal layer 650b together. For example, an electrical field may be applied across the bond interface at distal face 99 of the proximal layer 650a and/or the proximal face 98 of the distal layer 650b, while the proximal layer 650a and/or the distal layer 650b are heated. In an alternative embodiment, the proximal layer 650a and the distal layer 650b may be bonded together by using a laser-assisted bonding process, including applying localized heating to the distal face 99 of the proximal layer 650a and/or the proximal face 98 of the distal layer 650b to bond them together.

In some embodiments, as shown for example in FIG. 3A-FIG. 3F, the fluidic distribution block 650 may include a proximal layer 650a that includes supply channels 155a of the fluid distribution manifold and a distal layer 650b that includes resistance channels 155b of the fluid distribution manifold.

In various embodiments, the fluid distribution manifold includes a plurality of channels and/or apertures extending between the proximal face 151 and the distal face 97 of the fluidic distribution block 650. In some embodiments, each of the supply channels 155a is coupled in flow communication to a plurality of resistance channels 155b. In some embodiments, the resistance channels 155b extend away from the supply channels 155a and are configured to facilitate an increase in the resistance of the fluid distribution manifold to the flow of the fluid. Each resistance channel 155b may be coupled in flow communication to an outlet aperture 302. Each outlet aperture 302 may be aligned with an exit channel 155c of a microneedle 156 for distributing the fluid through the exit channels 155c. In some embodiments, the resistance channels 155b may be formed in any configuration that enables the fluidic distribution block 650 to function as described herein.

In some embodiments, the resistance channels 155b may have an axial and/or lateral internal diameter that is smaller than an axial and/or lateral internal diameter of the supply channels 155a. Moreover, the resistance channels 155b may be formed to create a tortuous flow path for the fluid, thereby facilitating an increase of the resistance of the fluid distribution manifold to the flow of the fluid.

Each microneedle 156 may define the one or more exit channels 155c in fluid connection with the fluid distribution manifold defined in the fluidic distribution block 650. In general, the exit channels 155c may be defined at any suitable location in each microneedle 156. For example, the exit channels 155c may be defined along an exterior surface of each microneedle 156. Alternatively and/or in addition, the exit channels 155c may be defined through the interior of the microneedles 156 such that each microneedle 156 forms a hollow shaft. The supply channels 155a, resistance channels 155b, and fluidic exit channels 155c may be configured to define any suitable cross-sectional shape. For example, each supply channel 155a, resistance channels 155b and/or fluidic exit channel 155c may define a semi-circular or circular shape, or a non-circular shape, such as a “v” shape or any other suitable cross-sectional shape. In some embodiments, the exit channel 155c of a microneedle 156 may terminate at an exit hole 157.

In some embodiments, for example as shown in FIG. 3G-FIG. 3J, each of the resistance channels 155b may include one or more inlet apertures 301 adapted to be coupled to and in fluidic connection with a supply channel 155a. Each of the resistance channels 155b may also include one or more outlet apertures 302 adapted to be coupled to and in fluidic connection with an exit channel 155c. Each exit channel 155c may extend through the base plate 300 as well as through the microneedle 156 (e.g., see FIG. 3F).

In some embodiments, for example as shown in FIG. 3B, the distal face 99 of the proximal layer 650a is adapted to be coupled to the proximal face 98 of the distal layer 650b and the distal face 97 of the proximal layer 650b is adapted to be coupled to the proximal face 96 of the base plate 300 such that the proximal entrance 154 of the microneedle fluidic block 150 is in flow communication through the fluid distribution manifold with the exit channels 155c of the microneedles 156. The proximal layer 650a, distal layer 650b and base plate 300 are adapted to form a sealed, leak-proof fluidic connection when coupled together. The proximal layer 650a and the distal layer 650b may be bonded together with an adhesive and/or the distal layer 650b and the base plate 300 may be bonded together with an adhesive or other bonding method described herein.

In some embodiments, for example as shown in FIG. 3A, when the proximal layer 650a, distal layer 650b and base plate 300 of the microneedle fluidic block assembly 150 are coupled together, the supply channels 155a, resistance channels 155b and exit channels 155c are adapted to be in fluidic connection and configured to allow a fluid to flow through the microneedle fluidic block assembly 150.

In some embodiments, the arrangement and dimensions of the supply channels 155a, resistance channels 155b and/or exit channels 155c of the microneedle fluidic block assembly 150 are configured to provide a suitable resistance against fluid movement such that the flow of fluid through each microneedle 156 is substantially the same, thereby substantially equally distributing the distal flow of fluid from the proximal entrance 154 to each of the microneedles 156 and to provide a suitable fluid flow rate through each of the microneedles 156.

In some embodiments, for example as shown in FIG. 3K, the proximal layer 650a may have an axial thickness of about 0.7 mm (e.g., see “(H)” in FIG. 3K). In some embodiments, for example as shown in FIG. 3K, the distal layer 650b may have an axial thickness of about 0.5 mm (e.g., see “(I)” in FIG. 3K).

As shown for example in FIG. 3K, in some embodiments, the proximal entrance 154 of the fluidic distribution block 650 may have an axially tapering or funnel-like shape. For example, in some embodiments, the proximal entrance 154 may have a lateral diameter of about 0.6 mm to 0.4 mm, e.g. about 0.50 mm, at the proximal face 151 of the proximal layer 650a of the fluidic distribution block 650 (e.g., see “(B)” in FIG. 3K) and may have a lateral diameter of about 0.15 mm to 0.35 mm, e.g. about 0.25 mm, at the distal face 99 of the proximal layer 650a of the fluidic distribution block 650 (e.g., see “(C)” in FIG. 3K). For example, in some embodiments, the outlet apertures 302 may have a lateral diameter of about 0.09 mm to 0.23 mm, e.g. about 0.16 mm, at the proximal face 98 of the distal layer 650b of the fluidic distribution block 650 (e.g., see “(D)” in FIG. 3K) and may have a lateral diameter of about 0.23 mm to 0.37 mm, e.g. about 0.3 mm, at the distal face 97 of the distal layer 650b of the fluidic distribution block 650 (e.g., see “(E)” in FIG. 3K).

In some embodiments, the supply channels may have an axial depth of about 40 μm (e.g., see “(G)” in FIG. 3K) and may have a lateral width of about 200 μm (e.g., see “(A)” in FIG. 3L).

In particular, in some embodiments, the dimensions of the resistance channels 155b may be varied to provide a range of fluid flow rates. It is to be understood that one or more of the length, axial depth and/or lateral width of a resistance channel 155b may be varied in any combinations. The distance along a resistance channel from an inlet aperture 301 to an outlet apertures 302 may define the length of a resistance channel 155b (e.g., see FIG. 3L). The depth of an example resistance channel 155b is shown in schematic form in FIG. 3K (see “(F)” in FIG. 3K). The width of an example resistance channel 155b is shown in schematic form in FIG. 3I and FIG. 3J (see “(X)” in FIG. 3I and FIG. 3J).

For example, in some embodiments, the length of a resistance channel 156 may be from about 400 m to about 1,000 μm, e.g. about 400 μm, 450 μm, 500 μm, 550 μm, 600 μm, 650 μm, 700 μm, 750 μm, 800 μm, 850 μm, 900 μm, 950 μm, or 1,000 μm. In some embodiments, the axial depth of a resistance channel 156 may be from about 10 μm to about 20 μm, e.g. about 10 μm, 11 μm, 12 μm, 13 μm, 14 μm, 15 μm, 16 μm, 17 μm, 18 μm, 19 μm, or 20 μm. In some embodiments, the lateral width of a resistance channel 156 may be from about 15 μm to about 70 μm, e.g. about 15 μm, 20 μm, 25 μm, 30 μm, 35 μm, 40 μm, 45 μm, 50 μm, 55 μm, 60 μm, 65 μm, or 70 μm. Various values of resistance channel length, depth and width may be used in any combinations.

For example, the Tables of FIG. 3K and FIG. 3L show various example combinations of resistance channel length, depth and width employed in example fluidic distribution blocks, as indicated for example microdose device designs D01 to D15. For each example design, the associated estimated fluid flow rate per microneedle is indicated, in response to forcing a fluid through the device using a syringe as described herein. For each of the example designs, the pressure of the fluid flowing through the microdose device is 0.7 bar (FIG. 3M) or 1.0 bar (FIG. 3N), and the estimated resistance ratio (Rchannel/Rmicroneedle) is indicated based on a microneedle having an exit channel having a length 400 μm and a width 40 μm. It is to be understood that the fluid flow rate per microneedle is multiplied by the number of microneedles per microdose device to provide the total fluid flow rate for the microdose device. For example, for a device having 16 microneedles, the estimated flow rates per microneedle shown in the Tables of FIG. 3M and FIG. 3N would be multiplied by 16. For example, in response to a axial fluid delivery force resulting in a pressure of 1.0 bar, a device having 16 microneedles and example dimensions as in design D07 as shown in the Table of FIG. 3N would provide a microdose device flow rate of 18,735 μL per hour per microneedle×16 microneedles=299,760 μL per hour, which equates to a total microdose flow rate of 83.27 μL per second for the example D07 design microdose device having 16 microneedles.

In some embodiments, for example as shown in FIG. 3I and FIG. 3J, the outlet apertures 302 may have a diameter of 0.04 mm (e.g., see “(Y)” in FIG. 3I and FIG. 3J). In the example resistance channels 155b shown in FIG. 3I and FIG. 3J, the outlet apertures 302 are placed apart at a distance of e.g. 0.9 mm, so as to be configured to couple to exit channels 155c of the example microneedle fluidic block assembly 150, such as shown in FIGS. 3B and 3C.

In some embodiments, the dimensions of the exit channels 155c may be selected to induce a capillary flow of a fluid delivered by the microdose device. Without limitation to theory, in some embodiments, the capillary pressure within an exit channel 155c may be inversely proportional to the cross-sectional dimension of the exit channel 155c and directly proportional to the surface energy of the subject fluid, multiplied by the cosine of the contact angle of the liquid at the interface defined between the liquid and the exterior channel. Thus, to facilitate capillary flow of the fluid through the microneedle fluidic block assembly 150, the cross-sectional width dimension of the exit channels 155c (e.g., the diameter of the exit channels 155c) may be selectively controlled, with smaller dimensions generally resulting in higher capillary pressures. For example, the cross-sectional width dimension of the exit channels 155c may be selected so that, with regard to the width of each exit channel 155c, the cross-sectional area of each exit channel 155c ranges from about 1,000 square microns (μm²) to about 125,000 μm², such as from about 1,250 μm² to about 60,000 μm², or from about 6,000 μm² to about 20,000 μm², or any other sub-ranges therebetween.

Further details of an example embodiment of the microneedle fluidic block assembly 150 are shown in FIG. 3C-FIG. 3J. In some embodiments, such as shown in the example microneedle fluidic block assembly 150 of FIG. 3C, the plurality of microneedles 156 may include an array of 16 microneedles 156 disposed on the distal face 152 of the base plate 300 in a square 4×4 arrangement. It is to be understood that other numbers of microneedles and/or other arrangements of the microneedles are contemplated, as described further herein.

The plurality of microneedles 156 may generally include any suitable number of microneedles 156 disposed on the base plate 300. For example, the number of microneedles 156 may range from about 10 microneedles per square centimeter (cm²) to about 1,500 microneedles per cm², such as from about 50 microneedles per cm² to about 1250 microneedles per cm², or from about 100 microneedles per cm² to about 500 microneedles per cm², or any other sub-ranges therebetween.

For example, in some embodiments, the plurality of microneedles may include from 2 to 100 microneedles, such as from 2 to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 microneedles.

The microneedles of plurality of microneedles described herein need not be identical to one another. A plurality of microneedles may have various lengths, outer diameters, inner diameters, cross-sectional shapes, nanotopography surfaces, and/or spacing.

The microneedles may be arranged as uniformly or non-uniformly or randomly spaced on the distal face 152 of the base plate 300, and may be disposed thereon in other arrangements, such as in a circle, triangle, cross, or other patterns. The microneedles 156 may generally be arranged on the base plate 300 in a variety of different patterns, and such patterns may be designed for any particular use. For example, in some embodiments, the microneedles 156 may be spaced apart in a uniform manner, such as in a rectangular or square grid or in concentric circles. In such embodiments, the spacing of the microneedles 156 may generally depend on numerous factors, including, but not limited to, the length and width of the microneedles 156, as well as the amount and type of fluid that is intended to be delivered through or along the microneedles 156.

In some embodiments, the spacing between each microneedles may be from about 1 μm to about 1500 μm, including each integer within the specified range. In some aspects, the spacing between each microneedle may be about 200 μm, about 300 m, about 400 μm, about 500 m, about 600 μm, about 700 μm, about 800 μm, about 900 m, about 1000 am, about 1100 am, about 1200 am, about 1300 am, about 1400 μm or about 1500 am. As used in this context, “about” means±50 am.

In the example shown in FIG. 3C, the microneedles 156 are uniformly spaced in a 4×4 square arrangement of four microneedles 156 in each of four parallel rows, wherein each row is spaced 0.9 mm apart (see “(A)” in FIG. 3C), and each microneedle 156 is spaced 0.9 mm apart from the nearest microneedle 156 e.g. in the same row (see “(A)” in FIG. 3C), and the total length of each row is 2.7 mm (see “(B)” and “(C)” in FIG. 3C). In some embodiments, microneedle density as referred to herein may be calculated by dividing the total number of microneedles by the total area of the base plate on which microneedles are disposed. For example, the total area of the example microneedle array of FIG. 3C is 2.7 mm×2.7 mm=7.29 square mm. Therefore, such an example microneedle array having a total of 16 microneedles disposed on an area of 7.29 square mm gives a microneedle density of about 2.2 microneedles per square mm. It is to be understood that in some embodiments, the microneedles 156 may be spaced closer together or further apart. In some embodiments, the microneedles 156 may be disposed on the base plate 300 at a density of about 0.01 to 7 microneedles per square mm. For example, in some embodiments, the microneedles 156 may be disposed on the base plate 300 at a density of about 0.01, 0.1, 0.5, 1, 1.5, 2.0, 2.5, 3.0, 3.5, 4.0, 4.5, 5.0, 5.5, 6.0, 6.5, or 7.0 microneedles per square mm. In some embodiments, the device may comprise one or more microneedles 156 disposed on the base plate 300. The example base plate 300 of FIG. 3C has a square shape with side lengths of 5.4 mm (see “(C)” in FIG. 3C). It is to be understood that in some embodiments, the base plate 300 may have a different shape and/or dimensions.

In some embodiments, such as shown in the example microneedle fluidic block assembly 150 of FIG. 3D, the distance between the proximal face 151 of fluidic distribution block 650 and the distal face 152 of the base plate 300 may be about 0.50 mm (e.g., see “(B)” in FIG. 3D).

In some embodiments, such as in FIG. 3E, showing a view of detail A of the example microneedle fluidic block assembly 150 of FIG. 3D, an axial distance between the base 158 of each microneedle 156, coupled to the distal face 152 of the base plate 300, and the tip 159 of each microneedle 156 may be about 0.4 mm.

FIG. 3F is a side view schematic of an example microneedle fluidic block assembly 150, showing a side view of combined proximal layer 650a, distal layer 650b and base plate 300 and a plurality of microneedles 156. In FIG. 3F, the supply channels 155a, as well as the resistance channels 155b, and the exit channels 155c are in fluidic connection, and shown as dashed lines. FIG. 3G is an axial plan view schematic through the combined proximal layer 650a, distal layer 650b, base plate 300 and plurality of microneedles 156 of the microneedle fluidic block assembly 150. FIG. 3H is an axial plan view schematic of the distal layer 650b of the fluidic distribution block 650, showing the resistance channels 155b.

In some embodiments, for example as shown in FIG. 3H, the resistance channels 155b may include inner resistance channels 155b (e.g., see circled and labeled “C” in FIG. 3H-FIG. 3J) located closer to the lateral center of the fluidic distribution block 650, and outer resistance channels 155b located further from the lateral center of the fluidic distribution block 650 (e.g., see circled and labeled “B” in FIG. 3H-FIG. 3J). In some embodiments, each exit channel 155c may be in fluidic connection with one resistance channel 155b. In some embodiments, two or more inner resistance channels 155b may be in fluidic connection with one inlet aperture 301 (see, e.g. FIG. 3H-FIG. 3J). In some embodiments, each outer resistance channel 155 may be in fluidic connection with one inlet aperture 301 (see, e.g. FIG. 3H-FIG. 3J).

In some embodiments, devices that comprise an array of microneedles adaptable for use with the microdose device herein are identifiable by skilled persons upon reading the present disclosure. Particular exemplary structures and devices are described in International Patent Application Publication Nos. WO 2014/188343, WO 2014/132239, WO 2014/132240, WO 2013/061208, WO 2012/046149, WO 2011/135531, WO 2011/135530, WO 2011/135533, WO 2014/132240, WO 2015/16821, and International Patent Applications PCT/US2015/028154 (published as WO 2015/168214 A1), PCT/US2015/028150 (published as WO 2015/168210 A1), PCT/US2015/028158 (published as WO2015/168215 A1), PCT/US2015/028162 (published as WO 2015/168217 A1), PCT/US2015/028164 (published as WO 2015/168219 A1), PCT/US2015/038231 (published as WO 2016/003856 A1), PCT/US2015/038232 (published as WO 2016/003857 A1), PCT/US2016/043623 (published as WO 2017/019526 A1), PCT/US2016/043656 (published as WO 2017/019535 A1), PCT/US2017/027879 (published as WO 2017/189258 A1), PCT/US2017/027891 (published as WO 2017/189259 A1), PCT/US2017/064604 (published as WO 2018/111607 A1), PCT/US2017/064609 (published as WO 2018/111609 A1), PCT/US2017/064614 (published as WO 2018/111611 A1), PCT/US2017/064642 (published as WO 2018/111616 A1), PCT/US2017/064657 (published as WO 2018/111620 A1), and PCT/US2017/064668 (published as WO 2018/111621 A1), all of which are incorporated by reference herein in their entirety.

Such devices include the SOFUSA® drug delivery platform (Sorrento Therapeutics, Inc., San Diego). For example, such devices include SOFUSA® DoseConnect™ devices (Sorrento Therapeutics, Inc., San Diego).

The disclosures of the specifications, claims and drawings of the following patent applications and patents related to devices, including lymphatic delivery devices, methods of providing the same, and methods of using the same for lymphatic administration are incorporated by reference in their entireties: U.S. Pat. Nos. 9,962,536 and 9,550,053, U.S. application Ser. Nos. 15/305,193, 15/305,206, 15/305,201, 15/744,346, 14/354,223, and International Patent Application No.'s PCT/US2017/027879, PCT/US2017/027891, PCT/US2016/043656, PCT/US2017/064604, PCT/US2017/064609, PCT/US2017/064642, PCT/US2017/064614, PCT/US2017/064657, PCT/US2017/064668, and U.S. Provisional Patent Application No. 62/678,601, filed May 31, 2018, U.S. Provisional Patent Application No. 62/678,592, filed May 31, 2018, and U.S. Provisional Patent Application No. 62/678,584, filed May 31, 2018, and International Application No. PCT/US2019/034736.

Accordingly, in some embodiments of the microdose device 10, for example as shown in FIG. 1A and FIG. 1B, the tubing connector 120, plenum 130, first gasket 140, and microneedle fluidic block assembly 150 are adapted to be coupled together to allow a fluid to flow through the microdose device 10 in a generally proximal to distal direction from within the tube 123 of the tubing connector 120, through the orifice 135 of the plenum 130, through the hole 144 of the first gasket 140, and through the entrance 154 of the microneedle fluidic block assembly 150, and the supply channels 155a, resistance channels 155b and outlet apertures 302 of the fluid distribution manifold and exit channels 155c of the microneedles 156.

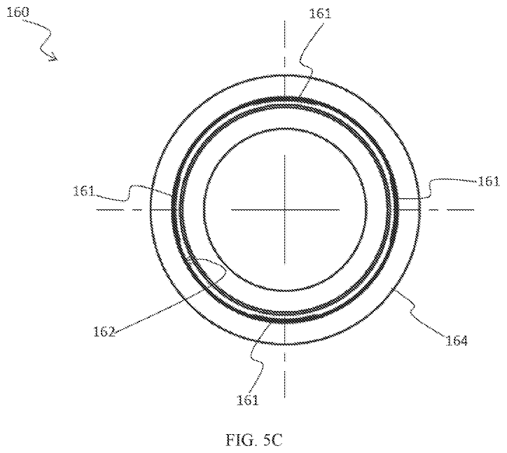

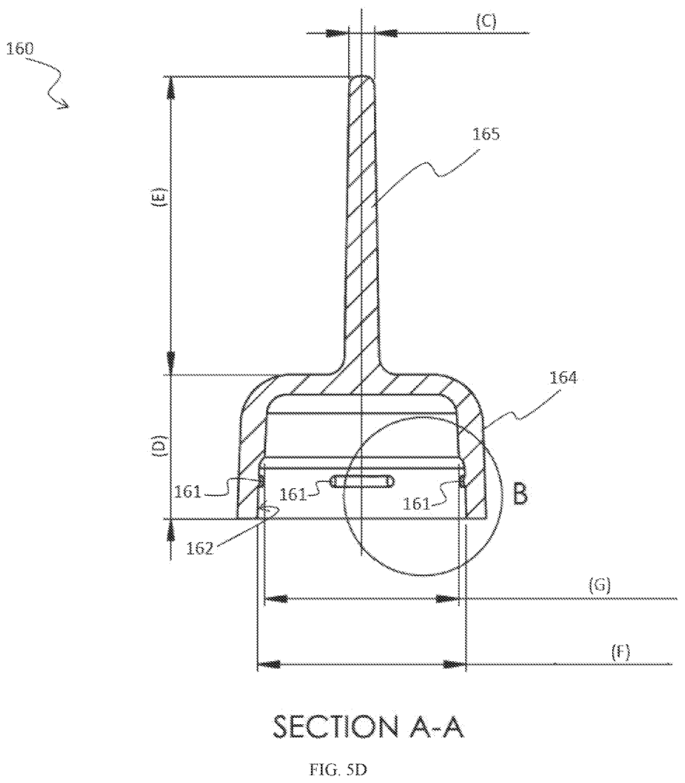

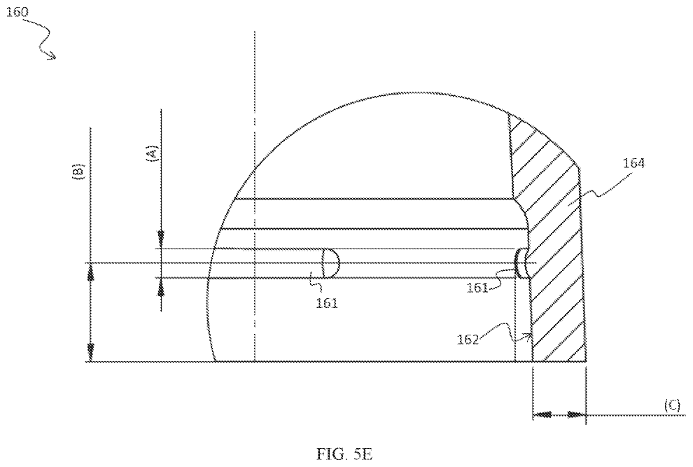

In some embodiments, for example as shown in FIG. 1A and FIG. 1B, the microdose device 10 may include a protective cap 160. The protective cap may be configured to provide a covering adapted to protect the physical integrity and/or sterility of the microdose device, in particular the physical integrity and/or sterility of the microneedle fluidic block assembly 150 and/or other components of the microdose device, until use. For example, the microdose device may be sterilized using methods known in the art, such as irradiation or chemical sterilization methods. The protective cap may be adapted to be coupled to the syringe In some embodiments, the protective cap 160 is adapted to be slidably coupled with the plenum 130 until the microdose device is to be used to administer a fluid composition to a patient. In some embodiments, for example as shown in FIG. 1D, the protective cap 160 may have one or more ridges 161 (e.g. four ridges 161 as shown in FIG. 5C) disposed on the inner surface 162 of the protective cap 160 that are adapted to engage with the plenum 130 such that ridges provide a friction fit adapted to provide a resistance against unintentional disengaging of the protective cap 160 from the plenum 130 such that the ridges 161 allow the protective cap to remain covering the plurality of microneedles 156 until the protective cap 160 is intentionally removed by a user. Typically, a proximal portion 163 of the protective cap 160 may include a cover 164 that is slidably coupled to the plenum 130. The protective cap 160 may have a tab 165 disposed thereon, for example at the distal end 166 of the protective cap 160, the tab 165 adapted to be grasped by user. The user may slidably attach the protective cap 160 to the plenum 130 by applying an axial force in a proximal direction until the ridges 161 of the protective cap 160 engage via a friction fit with the plenum 130, and the protective cap 160 may be slidably removed from the plenum 130 by application of an axial force in a distal direction to disengage the ridges 161 from the plenum 130 when a user intends to use the microdose device 10 to administer a fluid from the microdose device 10 to a patient.

In some embodiments, the axial length (from proximal end 163 to distal end 166) of the protective cap 160 may be about 22 mm (see, e.g. “(B)” indicated in FIG. 5B). In some embodiments, the lateral diameter of the cover 164 of the protective cap 160 may be about 12.5 mm (see, e.g. “(C)” indicated in FIG. 5B).

In some embodiments, the tab 165 may have a lateral thickness of about 1.3 mm (see e.g. “(C)” indicated in FIG. 5D). In some embodiments, the cover 164 may have an axial depth of about 7.1 mm (see e.g. “(D)” indicated in FIG. 5D). In some embodiments, the tab 165 may have an axial length of about 15 mm (see e.g. “(E)” indicated in FIG. 5D). In some embodiments, the cover 164 may have an inner diameter of about 10.4 mm (see e.g. “(F)” indicated in FIG. 5D). In some embodiments, each of the ridges 161 may extend laterally about 0.6 mm into the space inside the cover 164, such that when two ridges 161 are disposed on opposite sides of the inner surface 162 of the cover 164, the diameter between the two ridges may be about 9.8 mm (see e.g. “(G)” indicated in FIG. 5D). In some embodiments, the ridges 161 may have an axial thickness of about 0.6 mm (see e.g. “(A)” indicated in FIG. 5E) In some embodiments, the ridges 161 may be disposed on the inner surface 162 of the cover 164 at a distance of about 1.9 mm from the distal end 163 of the protective cap 160 (see e.g. “(B)” indicated in FIG. 5E). In some embodiments, the wall 169 of the cover 164 may have a thickness of about 1 mm (see e.g. “(C)” indicated in FIG. 5E). The protective cap 160 may be formed from any suitable material, e.g. a plastic material or other polymer, for example such as a cyclic olefin copolymer among others, having suitable flexibility to allow the cover 164 to flex enough to allow the ridges 161 to engage or disengage from the plenum 130 when user force is applied, as described above.

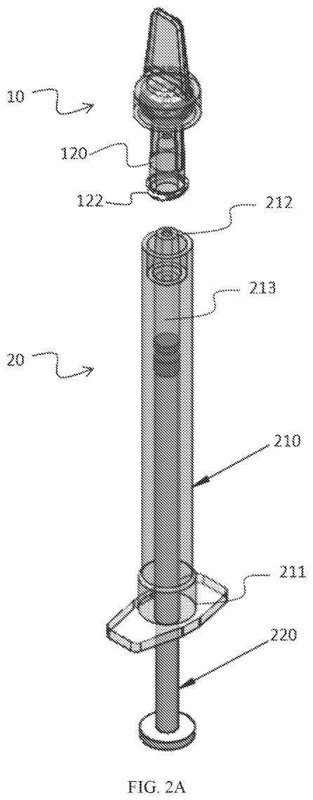

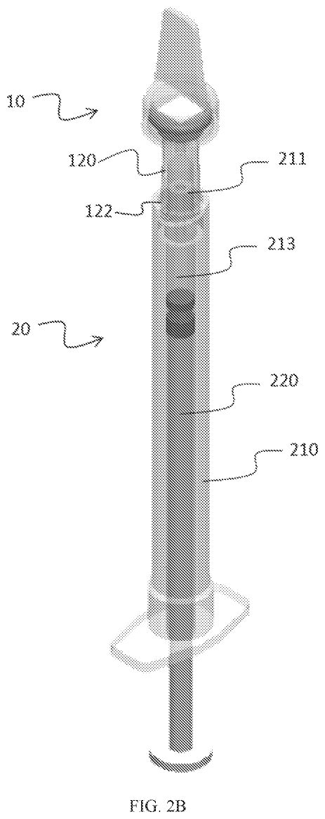

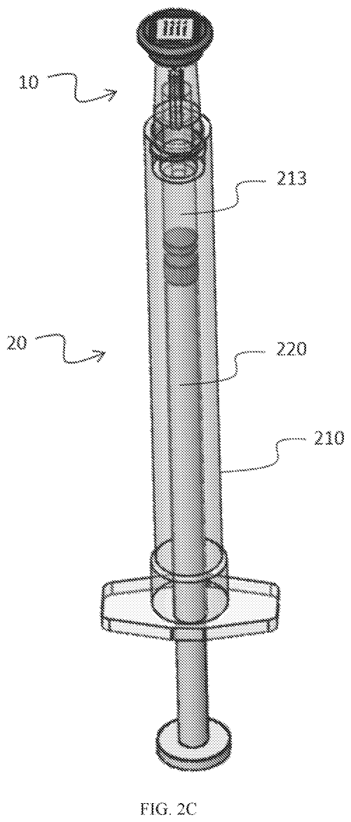

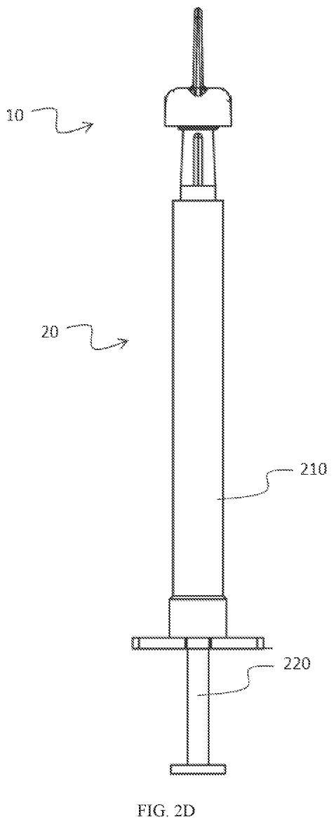

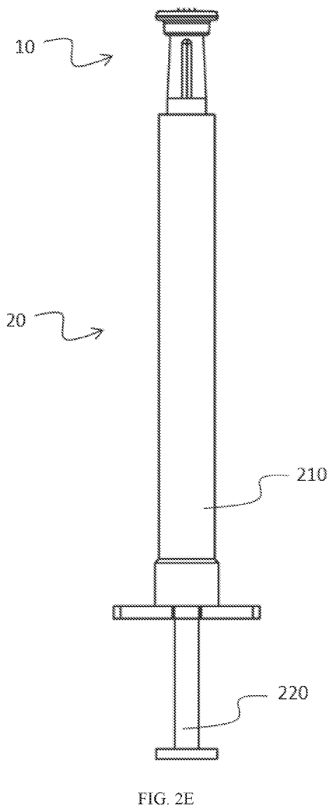

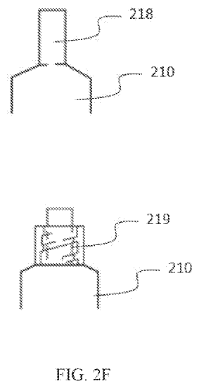

In some embodiments, the microdose device of the present disclosure configured for delivering a fluidic composition across a dermal barrier of a patient is adapted to be used in conjunction with a syringe. For example, FIG. 2A-FIG. 2E shows views of an example microdose device 10 and an example syringe 20, such as a 1 mL syringe. The syringe 20 has a barrel 210 having a bore defined therein, and a plunger 220 slidably coupled within the bore 213 of the syringe barrel 210. The barrel 210 has a proximal end 211 and a distal end 212. In response to an axial force applied to the plunger 220 in a distal direction, the plunger 220 moves axially within the bore 213 of the syringe barrel 210 towards the distal end 212 of the barrel 210 and causes fluid within the bore 213 to exit the distal end 212 of the syringe barrel 210. A proximal portion 122 of the syringe connection assembly 11, e.g., a proximal portion 122 of the example tubing connector 120 is adapted to be coupled to the distal end 212 of the syringe barrel 210, such that the bore 213 of the syringe barrel 210 and the fluidic path defined within the syringe connection assembly 11, e.g. the tube 123 of the example tubing connector 120, are in fluidic connection. The proximal portion 122 of the example tubing connector 120 and the distal end 212 of the syringe barrel 210 may be adapted to form a sealed, leak-proof fluidic connection when coupled together. The proximal portion 122 of the tubing connector 120 and the distal end 212 of the syringe barrel 210 may be adapted to maintain a sealed, leak-proof fluidic connection when fluid moves from the bore 213 of the syringe barrel 210 into the fluidic path defined within the syringe connection assembly 11, e.g. the tube 123 of the tubing connector 120.

Any suitable syringe 20 having a distal end 212 of its barrel 210 adapted to be coupled to the proximal portion 122 of the tubing connector 120 and capable of controllably ejecting a volume of a fluidic composition of from 1 μl to 500 μl may be used in conjunction with the microdose device of the present disclosure, such as syringes having a fluid volume capacity of the bore 213 of e.g., 1-10 μl, 10-50 μl, 50-100 μl, 100-500 μl, 500-1000 μl, 1.0-1.5 ml, or up to 2 ml, 5 ml, or 10 ml, for example.

In some embodiments, the syringe may be adapted to controllably eject the volume of a fluidic composition of from 1 μl to 500 μL over a period of time from about 0.1 second to about 5 minutes, such as up to 1, 2, 3, 4, 5, 10, 15, 20, 25, 30, 35, 40, 45, 50, 55, or 60 seconds, or up to 90, 120, 180, 210, 240, 270, or 300 seconds. The rate of ejection of the fluid from the syringe may be constant or variable, such as an increasing rate, or a decreasing rate, or a pulsatile rate, or any combinations thereof.

In some embodiments, the syringe 20 may further comprise a fluid within the bore 213, such as any fluid described herein. A syringe coupled to a microdose device may be provided to a user such that the syringe bore is pre-loaded with a fluid, or a user may fill a syringe with a fluid and attach the syringe to a microdose device.

Accordingly, in some embodiments, when the microdose device 10 of the present disclosure is coupled to a syringe barrel 210 of a syringe 20, in response to an axial force applied to the plunger 220 in a distal direction, a fluid in the bore 213 of the barrel 210 of the syringe 20 flows from the syringe 20, and the microdose device is configured such that the fluid flows through the microdose device 10 and exits the fluidic exit channels 155c of the plurality of microneedles 156 of the microdose device 10.

In some embodiments, the proximal portion 122 of the syringe connection assembly 11, e.g. the proximal portion 122 of the example tubing connector 120 and the distal end 212 of the syringe barrel 210 may be adapted to be slidably coupled together. In some embodiments, the proximal portion 122 of the proximal portion 122 of the syringe connection assembly 11, e.g. the proximal portion 122 of the tubing connector 120, may be adapted to be coupled to the distal end 212 of the syringe barrel 210 by compatible a screw threads. In such embodiments, the proximal portion 122 of the syringe connection assembly 11, e.g. the proximal portion 122 of the tubing connector 120, may have a screw thread disposed thereon (e.g., see an example screw thread 124 in FIG. 1A and FIG. 1C) that is compatible with a screw thread disposed on the distal end 212 of the syringe barrel 210.

In some embodiments, suitable syringes include, without limitation, BD Luer slip-tip or BD Luer-Lok® (Becton, Dickinson And Company Corporation New Jersey) syringes. For example, BD Luer slip-tip syringes, and other similar syringes, have a distal end 212 adapted for a friction-fit connection 218 that allows the proximal portion 122 of the tubing connector 120 to be slidably coupled to the distal end 212 of the syringe barrel 210. In another example, BD Luer-Lok® syringes, and other similar syringes, have a screw thread 219 disposed on the distal end 212 of the syringe barrel 210 (see, e.g. FIG. 2F). In several embodiments, the microdose device is adapted to be attached to existing syringes, such as a standard 0.5 mL or 1.0 mL volume syringe, e.g. 0.5 mL or 1.0 mL BD Luer, BD Luer-Lok® syringes.

Suitable syringes are commercially available from suppliers such as Becton, Dickinson And Company, or Hamilton, among others identifiable by skilled persons upon reading the present disclosure. Suitable syringes may be sterile packaged, or may be sterilized, such as using an autoclave, or using chemical sterilization methods or other methods known in the art.

In use, in some embodiments, the microdose device coupled to a syringe may be configured to be hand-held and/or may be coupled to a syringe pump device adapted to controllably advance the plunger through the barrel. Such syringe pump devices include, without limitation, commercially available syringe pumps such as those from Harvard Apparatus, among others identifiable by skilled persons upon reading the present disclosure.

In some embodiments, desired fluid delivery rates as used herein may be initiated by driving the fluidic composition described herein with the application of pressure or other driving means, including pumps, elastomer membranes, gas pressure, piezoelectric, electromotive, electromagnetic, peristaltic or osmotic pumping, or use of rate control membranes or combinations thereof.

In some embodiments, the microdose device described herein may be coupled to a syringe as described herein and may be configured to be held in a hand of a user and used in a simple and straightforward manner to manually administer a bolus injection of a fluidic composition (for example, without limitation, having a total volume of up to 500 μL, e.g. 100 μL) across a dermal barrier of a patient in a short period of time (for example, without limitation, up to 300 seconds, e.g. 10 seconds). In contrast, existing devices such as SOFUSA® DoseConnect™ devices (Sorrento Therapeutics, Inc., San Diego) are typically configured to deliver an infusion of a fluidic composition across the dermal barrier of a patient over a longer period of time. In addition, in some embodiments, the administering of a fluidic composition using the microdose device described herein may be performed without the aid of one or more additional components such as those described for use with the SOFUSA® DoseConnect™ devices (Sorrento Therapeutics, Inc., San Diego), for example as described in U.S. Prov. App. No. 62/942,971, such as one or more of collet assembly, a controller assembly, an applicator, or an attachment band. As described for example in U.S. Prov. App. No. 62/942,971, a collet assembly is configured to contact a surface of the patient's skin sufficient for penetration of the plurality of microneedles into the surface of the patient's skin and across the dermal barrier; a controller assembly is configured to control the flow of the fluidic composition during delivery of the fluidic composition through the plurality of microneedles; an applicator is configured to facilitate the transition of the microdose device from a non-activated configuration to an activated configuration; and an attachment band assembly is configured to facilitate contact with a surface of the subject's skin sufficient for penetration of the plurality of protrusions into the surface of the subject's skin and across the dermal barrier. For example, as evidenced by example results described in Example 5 and Example 1 of the present disclosure, in use, the microdose device described herein may be configured to be held in a hand of a user and operable in a simple and straightforward manner, e.g. without the aid of one or more of a collet assembly, a controller assembly, an applicator, or an attachment band, to achieve penetration of the plurality of protrusions into the surface of the subject's skin and across the dermal barrier and to deliver a fluidic composition across a dermal barrier of a patient, e.g. to the lymphatic system of a patient.

In some embodiments, the microdose device is adapted to have a leak-proof fluidic connection between the bore of the syringe and the exit channels of the microneedles, such that in use, when an axial force is applied to the plunger of the syringe, fluid flows through the microdose device and exits the exit channels of the microneedles without leakage or loss of the fluid from any of the couplings between the components of the microdose device or between the microdose device and the syringe. As described in Example 4, the microdose device of the present disclosure is adapted to perform in a leak-proof manner when used in the methods described herein.

FIG. 6A to FIG. 8E of the present disclosure illustrate a second example microdose device and components thereof.

In FIG. 6A, a perspective exploded view schematic is shown of an example second set of components adapted to be combined to form a microdose device 10 of the present disclosure, as shown in FIG. 6B. The components shown in FIG. 6A include a syringe connection assembly 11 comprising a tubing connector 120 and a plenum 130. The components of the example microdose device 10 shown in FIG. 1 also include a first gasket 140, a microneedle fluidic block 150 comprising a fluidic block 650, a second gasket 640, and a microneedle array 660. The components of the example microdose device 10 shown in FIG. 6A also include a protective cap 160.

As would be understood from the example drawings in FIG. 6A and FIG. 6B, in some embodiments, a microdose device of the present disclosure, such as the example microdose device 10 shown in FIG. 6B, may be assembled from the example components shown in FIG. 6A as described herein.

The second example microdose device 10 illustrated in FIG. 6A to FIG. 10F differs from the first example microdose device 10 illustrated in FIG. 1A to FIG. 5E for example in that the microneedle fluidic block 150 of the second example microdose device 10 includes a second gasket 640 disposed between the fluidic block 650 and the microneedle array 660. Configuration and assembly together of the other components of the example second example microdose device 10 illustrated in FIG. 6A-FIG. 10F, including the tubing connector 120, plenum 130, first gasket 140, and protective cap 160, is similar to that as described herein for the first example microdose device illustrated in FIG. 1A to FIG. 5E.

As shown for example in FIG. 6A, the fluidic distribution block 650 has a proximal face 151 and a distal face 97. In the second example microdose device 10 illustrated in FIG. 6A, the distal face 97 of the fluidic distribution block 650 is adapted to be coupled to the proximal face 641 of the second gasket 640. The distal face 642 of the second gasket 640 is adapted to be coupled to the proximal face 96 of the base plate 300 of the microneedle array 660.

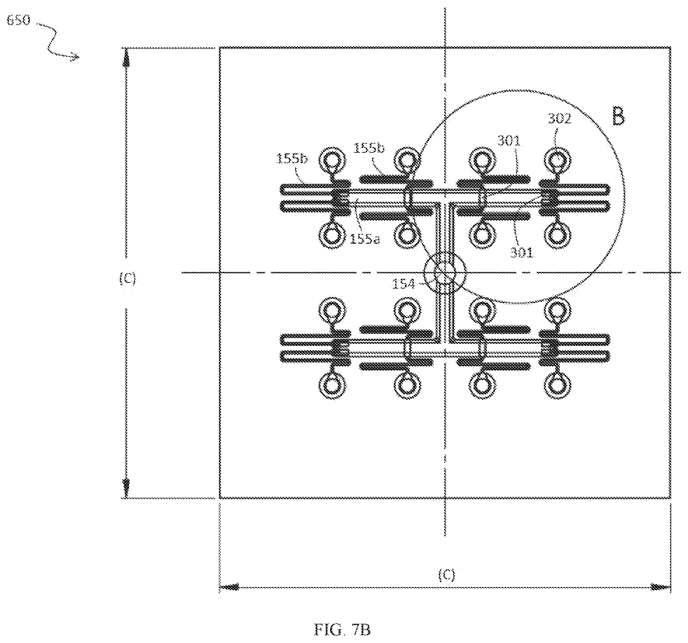

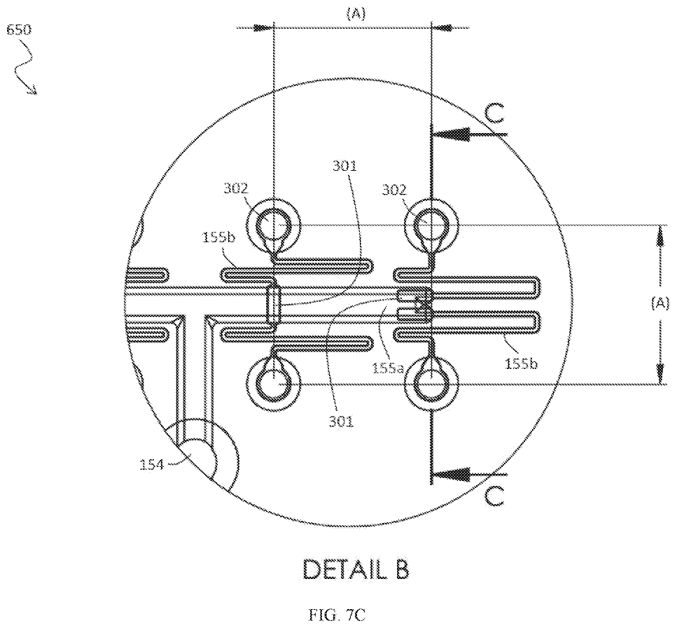

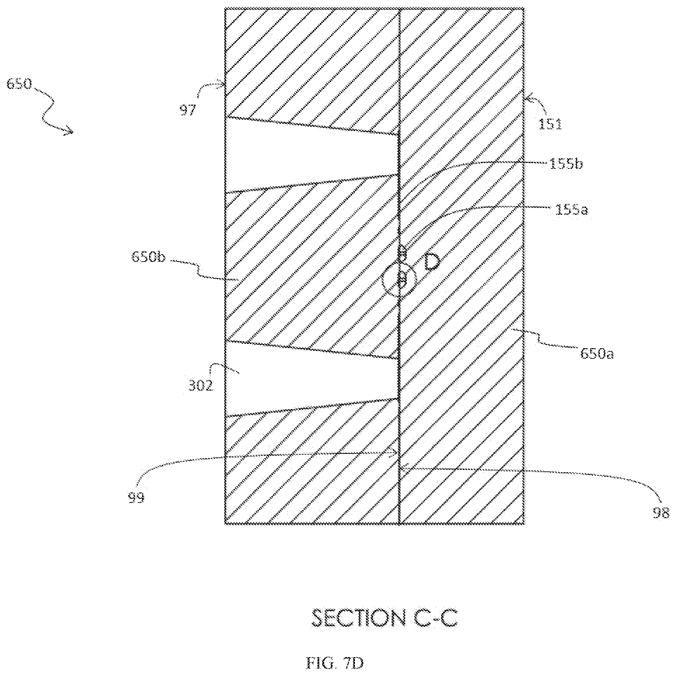

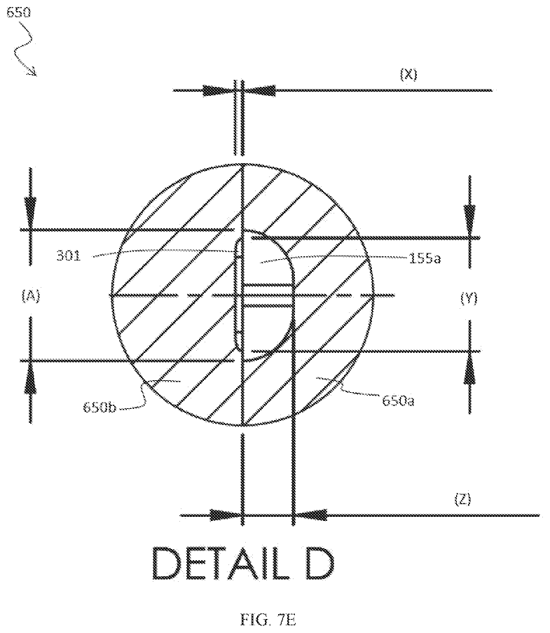

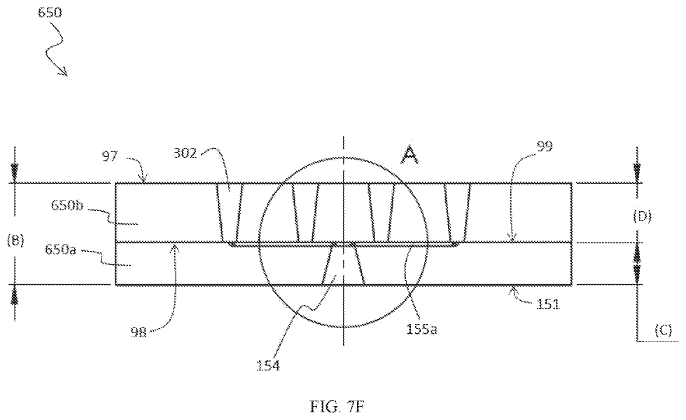

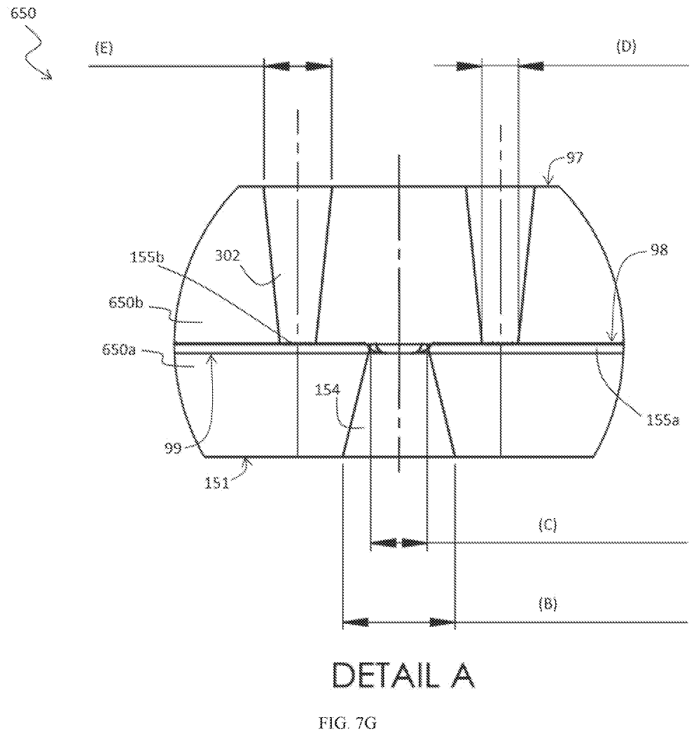

As shown for example in FIG. 6A, FIG. 6D and FIG. 6E, the fluidic distribution block 650 (including a proximal layer 650a and a distal layer 650b of the fluidic distribution block 650; see e.g. FIG. 7A) may be sized such that the lateral edges 653 of the fluidic distribution block 650 are adapted to be coupled to the inner wall 138 of the distal seat 137 disposed in the distal face 131 of the plenum 130. Furthermore, the base plate 300 of the microneedle array 660 may be sized such that the lateral edges 663 of the base plate 300 of the microneedle array 660 are also adapted to be coupled to the inner wall 138 of the distal seat 137 disposed in the distal face 131 of the plenum 130.

The lateral edges 653 of the fluidic distribution block 650 and/or the lateral edges 663 of the base plate 300 of the microneedle array 660 may be adhered to the inner wall 138 of the distal seat 137 disposed in the distal face 131 of the plenum 130, for example using an adhesive such as Loctite 3979, or other suitable adhesive identifiable by skilled persons. An adhesive may be applied, e.g. in a continuous bead, between the inner wall 138 of the distal seat 137 disposed in the distal face 131 of the plenum 130 and the lateral edges 653 of the fluidic distribution block 650 and/or the lateral edges 663 of the base plate 300 of the microneedle array 660 (e.g., see adhesive 90 in FIG. 6E).

The example fluidic distribution block 650 is shown in more detail in FIG. 6E and FIG. 7A-FIG. 7G.

The fluidic distribution block 650 has a proximal entrance 154 forming an opening in the proximal face 151 of the fluidic distribution block 650 (see, e.g., FIG. 6E). In some embodiments, the proximal entrance 154 is adapted to allow a fluid to flow from the hole 144 of the first gasket 140 into the fluid distribution manifold including the supply channels 155a, resistance channels 155b and outlet apertures 302 disposed within the fluidic block 650.

In some embodiments, for example as shown in FIG. 7A-FIG. 7G, the fluidic distribution block 650 may include a proximal layer 650a that includes supply channels 155a of the fluid distribution manifold, and a distal layer 650b that includes resistance channels 155b of the fluid distribution manifold and a plurality of outlet apertures 302, each outlet aperture 302 extending axially through the distal layer 650b to the distal face 97 of the fluidic distribution block 650. Alternatively, in some embodiments, the fluidic distribution block 650 may include a proximal layer 650a that includes resistance channels 155b, and a distal layer 650b that includes supply channels 155a and a plurality of outlet apertures 302, each outlet apertures 302 extending axially through the distal layer 650b to the distal face 97 of the fluidic distribution block 650.

The distal face 99 of the proximal layer 650a may be coupled, e.g. adhered, to the proximal face 98 of the distal layer 650b, such that the proximal entrance 154, supply channels 155a, resistance channels 155b and outlet apertures 302 are adapted to be in fluidic connection and configured to allow a fluid to flow through the fluid distribution manifold of the fluidic distribution block 650.

It is to be understood that any suitable configuration of the fluid distribution manifold may be employed in the fluidic block described herein, wherein the proximal entrance 154, supply channels 155a, resistance channels 155b and outlet apertures 302 are adapted to be in fluidic connection and configured to allow a fluid to flow through the fluidic distribution block.

The proximal layer 650a and distal layer 650b are adapted to form a sealed, leak-proof fluidic connection when coupled together. The proximal layer 650a and distal layer 650b may be bonded together using an adhesive.

In some embodiments, for example as shown in FIG. 7A-FIG. 7C, each of the resistance channels 155b may include one or more inlet apertures 301 adapted to be coupled to and in fluidic connection with a supply channel 155a.

The example fluidic block 650 of FIG. 7B has a square shape with side lengths of 5.4 mm (see “(C)” in FIG. 7B). It is to be understood that in some embodiments, the fluidic block 650 may have a different shape and/or dimensions.

In the example resistance channels 155b shown in FIG. 7B and FIG. 7C, the outlet apertures 302 may be placed apart at a distance of e.g. 0.9 mm (see e.g. as indicated by “(A)” in FIG. 7C), so as to be configured to be coupled to and in fluidic connection with the exit channels 155c.

In some embodiments, for example as shown in FIG. 7E, the inlet apertures 301 of the resistance channels 155b may have an axial depth (e.g., see “(X)” in FIG. 7E) having dimensions as described herein, such as in FIG. 3M and FIG. 3N, and may have a lateral width (e.g., see “(Y)” in FIG. 7E) having dimensions as described herein, such as in FIG. 3M and FIG. 3N. In some embodiments, for example as shown in FIG. 7E, the supply channel 155a may have an axial depth (e.g., see “(Z)” in FIG. 7E) having dimensions as described herein, and may have a lateral width (e.g., see “(A)” in FIG. 7E) having dimensions as described herein.

In some embodiments, for example as shown in FIG. 7F, the fluidic distribution block 650 may have an axial thickness of about 1.2 mm (e.g., see “(B)” in FIG. 7F). In some embodiments, for example as shown in FIG. 7F, the proximal layer 650a may have an axial thickness of about 0.5 mm (e.g., see “(C)” in FIG. 7F). In some embodiments, for example as shown in FIG. 7F, the distal layer 650b may have an axial thickness of about 0.7 mm (e.g., see “(D)” in FIG. 7F).

As shown for example in FIG. 7G, in some embodiments, the proximal entrance 154 of the fluidic distribution block 650 may have an axially tapering or funnel-like shape. For example, in some embodiments, the proximal entrance 154 may have a lateral diameter at the proximal face 151 of the proximal layer 650a of the fluidic distribution block 650 (e.g., see “(B)” in FIG. 7G) having dimensions as described herein, and may have a lateral diameter at the distal face 99 of the proximal layer 650a of the fluidic distribution block 650 (e.g., see “(C)” in FIG. 7G) having dimensions as described herein. In some embodiments, the outlet apertures 302 may have a lateral diameter at the proximal face 98 of the distal layer 650b of the fluidic distribution block 650 (e.g., see “(D)” in FIG. 7G) having dimensions as described herein, and may have a lateral diameter at the distal face 97 of the distal layer 650b of the fluidic distribution block 650 (e.g., see “(E)” in FIG. 7G) having dimensions as described herein.

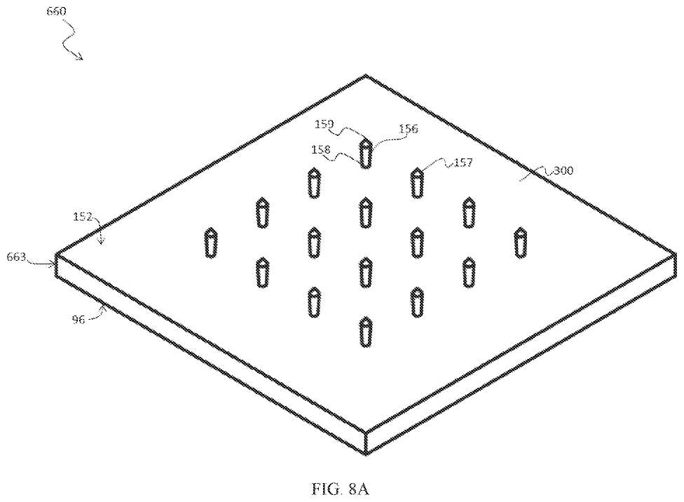

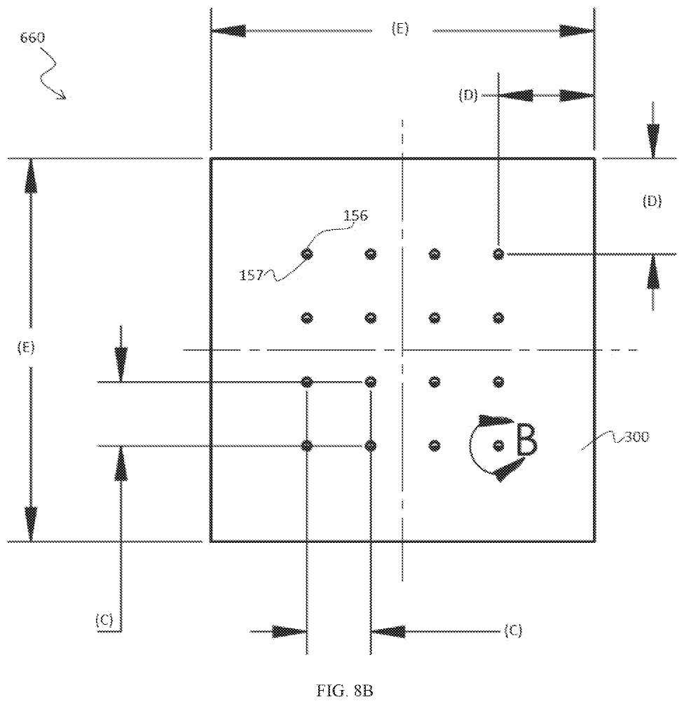

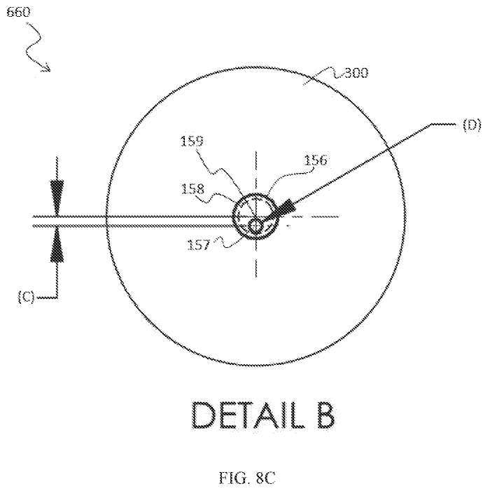

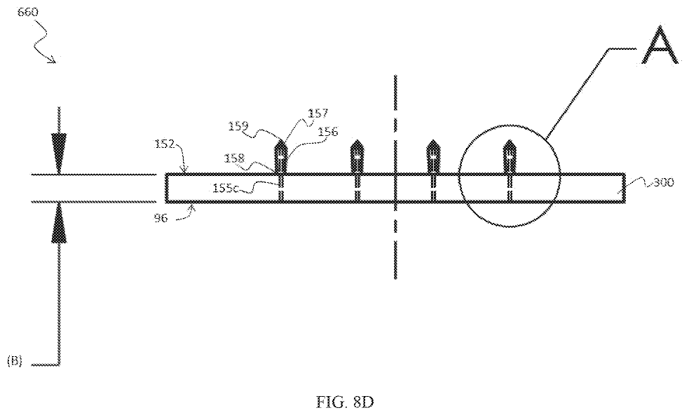

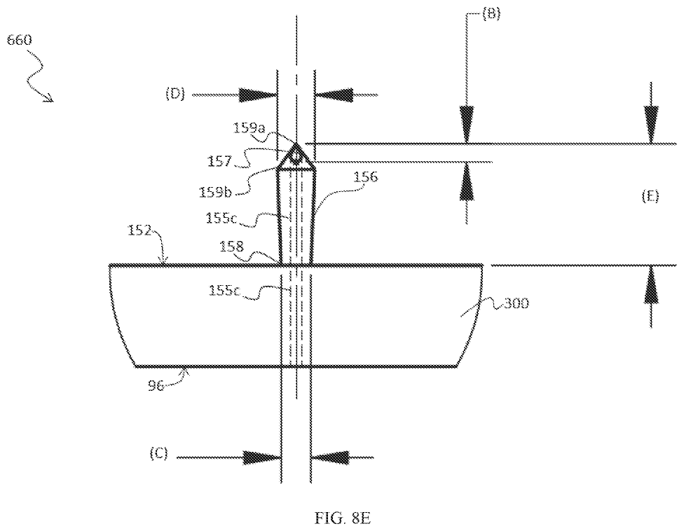

Further details of an example microneedle array 660 are shown in FIG. 8A-FIG. 8E. The microneedle array 660 includes a base plate 300 having a proximal face 96 and a distal face 152. The microneedle array 660 also has a plurality of microneedles 156 disposed on the distal face 152 of the base plate 300. Each of the microneedles 156 has a base 158 coupled to the distal face 152 of the base plate 300 and extends away distally from the base plate 300 to a distal end comprising a tip 159. The tip 159 may have a piercing or needle-like shape, such as a conical or pyramidal shape or a cylindrical shape transitioning to a conical or pyramidal shape. In some embodiments, the exit channel 155 of a microneedle 156 may terminate at an exit hole 157.

Similar to the first example microdose device 10 (e.g., see FIG. 3C), in the example second microdose device 10, the example microneedle array 660 shown in FIG. 8B has an array of 16 microneedles 156 disposed on the distal face 152 of the base plate 300 in a square 4×4 arrangement. It is to be understood that other numbers of microneedles and/or other arrangements of the microneedles are contemplated. In the example shown in FIG. 8B, the microneedles 156 are uniformly spaced in a 4×4 square arrangement of four microneedles 156 in each of four parallel rows, wherein each row is spaced 0.9 mm apart (see “(C)” in FIG. 8B), and each microneedle 156 is spaced 0.9 mm apart from the nearest microneedle 156 in the same row (see “(C)” in FIG. 8B), and the microneedles 156 closest to the lateral edges 663 of the microneedle array 660 are about 1.35 mm from the lateral edge 663 (see “(D)” in FIG. 8B). It is to be understood that in some embodiments, the microneedles 156 may be spaced closer together or further apart. The example base plate 300 of FIG. 8B has a square shape with side lengths of 5.4 mm (see “(E)” in FIG. 8B). It is to be understood that in some embodiments, the base plate 300 may have a different shape and/or dimensions.

Further details of the example microneedle array 660 of FIG. 8B is shown in FIG. 8C-FIG. 8E. In some embodiments, such as for example in FIG. 8C, the exit hole 157 may be disposed within the tip 159 of the microneedle 156 such that it is positioned off-center. For example, in FIG. 8C, the lateral center of the diameter of the exit hole 159 is positioned about 0.025 mm from the lateral center of the diameter of the microneedle 156 (e.g., see “(C)” in FIG. 8C). In some embodiments, the lateral diameter of the exit hole 157 may be about 0.035 mm (e.g., see “(D)” in FIG. 8C). In some embodiments, for example as shown in the example microneedle 156 in FIG. 8C, the microneedle base 158 (shown as a dashed line) has a smaller lateral diameter than the widest part of the microneedle tip 159, shown as a solid circle around the dashed circle of the base 158 (see also FIG. 8E).

In some embodiments, an outlet aperture 302 of the fluidic distribution block 650 is configured to be in fluidic connection with an exit hole 157 of a microneedle 156 via an exit channel 155c extending from the proximal face 96 of the base plate 300 of the microneedle array 660 to the exit hole 157, for example as shown in FIG. 8D. In some embodiments, for example such as shown in FIG. 8D, the axial thickness of the base plate 300 may be about 0.325 mm (see e.g. “(B)” indicated in FIG. 8D). As shown in further detail in FIG. 8E, in some embodiments, the axial distance from the distal end of the tip 159a to the proximal end of the exit hole 157 may be about 0.079 mm (e.g., see “(B)” in FIG. 8E). In some embodiments, the microneedle base 158 may have a smaller lateral diameter than the widest part of the microneedle tip 159b. For example, the microneedle base 158 may have a lateral diameter of about 0.08 mm (e.g., see “(C)” in FIG. 8E) and the widest part of the microneedle tip 159b may have a lateral diameter of about 0.14 mm (e.g., see “(D)” in FIG. 8E). In some embodiments, the microneedles 156 may have an axial length of about 0.39 mm (e.g., see “(E)” in FIG. 8E).

In some embodiments, the fluidic distribution block 650 and/or the microneedle array 660 may be formed from any suitable material, e.g. a suitable polymer or plastic material, for example and without limitation a polymethyl methacrylate, or a silica or glass material or the like, for example and without limitation a borosilicate glass (e.g. MEMpax®, Schott, Germany), among others.

In some embodiments, the proximal face 96 of the base plate 300 of the microneedle array 660 may be coupled to the distal face 642 of the second gasket 640 and the distal face 97 of the fluidic distribution block 650 may be coupled to the proximal face 641 of the second gasket 640.

The second gasket 640 may be a pressure-sensitive adhesive (PSA) gasket. The second gasket 640 may have an adhesive disposed on the proximal face 641 of the second gasket 640 and/or the distal face 642 of the second gasket 640. The second gasket 640 has a plurality of holes 646 adapted to allow a fluid to flow through the second gasket 640 from the proximal face 641 of the second gasket 640 to the distal face 642 of the second gasket 640. When the second gasket 640 is coupled to the base plate 300 of the microneedle array 660 and the fluidic distribution block 650, the holes 646 are adapted to allow a fluid to flow from the plurality of outlet apertures 302 through the holes 646 of the second gasket 640 into the exit channels 155c of the microneedle array 660.

Accordingly, in some embodiments, when the microdose device 10 of the present disclosure is coupled to a syringe 20, in response to an axial force applied to the plunger 220 in a distal direction, a fluid in the bore 213 of the barrel 210 of the syringe 20 flows from the syringe 20, and the microdose device is configured such that the fluid flows through the microdose device 10 and out of the exit channels 155c of the plurality of microneedles 156 of the microneedle array 660.

In some embodiments, the microneedle array may be a draped microneedle array. An example draped micro-needle array 900 is shown in FIG. 9A to FIG. 9E. For example, as shown in FIG. 9A to FIG. 9B, in some embodiments, the draped microneedle array 900 may include a microneedle array 660, a third gasket 1000, and a film 1100. The film 1100 may have a nanopatterned layer disposed thereon, such that the draped microneedle array includes a nanopatterned layer, also referred to herein as a nanotopography, the film draped at least partially across the plurality of microneedles 156 and optionally also draped at least partially across the base plate 300 of the microneedle array 660.

In some embodiments, for example as shown in FIG. 9B, the distal face 152 of the base plate 300 of the microneedle array 660 is adapted to be coupled to the proximal face 1001 of the third gasket 1000 and the distal face 1002 of the third gasket 1000 is adapted to be coupled to the proximal face 1101 of the film 1100.

The third gasket 1000 may be a pressure-sensitive adhesive (PSA) gasket. In some embodiments, the third gasket 1000 may comprise a PSA layer provided between the nanopatterned layer and the surface of the plurality of microneedles, providing support. The PSA layer may be formed from an adhesive material (e.g., ARcare® 93445).

The third gasket 1000 may have an adhesive disposed on the proximal face 1001 of the third gasket 1000 and/or the distal face 1002 of the third gasket 1000. The third gasket 1000 has a plurality of holes 1046 adapted to allow the plurality of microneedles 156 of the microneedle array 660 to be disposed through the plurality of holes 1046 when the distal face 152 of the base plate 300 of the microneedle array 660 is coupled to the proximal face 1001 of the third gasket 1000.

In some embodiments, when the distal face 152 of the base plate 300 of the microneedle array 660 is coupled to the proximal face 1001 of the third gasket 1000, the film 1100 may be coupled to the distal face 152 of the base plate 300 of the microneedle array 660 and the distal face 1002 of the third gasket 1000 such that the film 1100 forms a drape over the plurality of microneedles 156, for example as shown in FIG. 9A to FIG. 9D and in further detail in FIG. 9E. The film 1100 may include film exit holes 1103 in fluidic connection with the exit holes 157 of the microneedles 156 and configured to allow a fluid to flow therethrough, such that the draped microneedle array 900 is adapted to allow a fluid to flow from the exit channels 155c of the microneedle array 660 and through the exit holes 157 of the microneedles 156 and also through the film exit holes 1103.

In some embodiments, the nanopatterned layer may comprise a plurality of nanostructures and covering a surface of the plurality of microneedles 156. In some embodiments, the nanostructures comprise a height and a cross-sectional dimension. In some embodiments, at least a portion of the nanostructures have center-to-center spacing of from about 50 nanometers to about 1 micrometer. In some embodiments, at least a portion of the nanostructures have a height of from about 10 nanometers to about 20 micrometers. In some embodiments, at least a portion of the nanostructures have an aspect ratio of the height to the cross-sectional dimension from about 0.15 to about 30. In some embodiments, the nanostructures constitute a nanopattern having a fractal dimension of greater than about 1. In some embodiments, at least a portion of the nanostructures have a surface comprising a plurality of nanostructures having an average surface roughness ranging from about 10 nm to about 200 nm. In some embodiments, at least a portion of the nanostructures have an effective compression modulus ranging from about 4 MPa to about 320 MPa. In some embodiments, the microneedle array 660 comprises a nanopatterned layer comprising a plurality of nanostructures having one or more of the above described characteristics.

In some embodiments, the nanopatterned layer further comprises a plurality of additional nanostructures having a cross-sectional dimension less than the cross-sectional dimension of the nanostructures.

In some embodiments, the nanopatterned layer may be fabricated from a polymeric film, or the like, and coupled to the fluid distribution assembly using an additional adhesive layer. In other embodiments, the film may include an embossed or nano-imprinted, polymeric (e.g., plastic) film, or a polyether ether ketone (PEEK) film, or any other suitable material, such as a polypropylene film.

A further example of a fluidic distribution block 650 of the present disclosure is shown FIG. 10A to FIG. 10H.

The example fluidic distribution block 650 shown in FIG. 10A to FIG. 10H has an alternative configuration of the supply channels 155a and resistance channels 155b as compared to the example fluidic block 650 illustrated in FIG. 7A to FIG. 7G, such that in the example fluidic distribution block 650 illustrated in FIG. 10A to FIG. 10H the proximal layer 650a includes the resistance channels 155b, and the distal layer 650b includes the supply channels 155a.

The example fluidic distribution block 650 of FIG. 10B has a square shape with side lengths of 5.4 mm (see “(C)” in FIG. 10B). It is to be understood that in some embodiments, the fluidic block 650 may have a different shape and/or dimensions.

In the example resistance channels 155b shown in FIG. 10B and FIG. 10C, the outlet apertures 302 may be placed apart at a distance of e.g. 0.9 mm (see e.g. as indicated by “(C)” in FIG. 10C), so as to be configured to be coupled to and in fluidic connection with the exit channels 155c, such as shown in FIG. 10A-FIG. 10C.

In some embodiments, for example as shown in FIG. 10E and FIG. 10F, the inlet apertures 301 may have an axial diameter of about 0.0037 mm (e.g., see “(X)” in FIG. 10F) and may have a lateral diameter of about 0.0284 mm (e.g., see “(Y)” in FIG. 10F). In some embodiments, for example as shown in FIG. 10E, the supply channel 155a may have an axial depth (e.g., see “(Z)” in FIG. 10E) having dimensions as described herein, and may have a lateral width (e.g., see “(A)” in FIG. 10E) having dimensions as described herein.

In some embodiments, for example as shown in FIG. 10G, the fluidic distribution block 650 may have an axial thickness of about 1.2 mm (e.g., see “(B)” in FIG. 10G). In some embodiments, for example as shown in FIG. 10G, the proximal layer 650a may have an axial thickness of about 0.7 mm (e.g., see “(C)” in FIG. 10G). In some embodiments, for example as shown in FIG. 10G, the distal layer 650b may have an axial thickness of about 0.5 mm (e.g., see “(D)” in FIG. 10G).

As shown for example in FIG. 10H, in some embodiments, the proximal entrance 154 of the fluidic block 650 may have an axially tapering or funnel-like shape. For example, in some embodiments, the proximal entrance 154 may have a lateral diameter at the proximal face 151 of the proximal layer 650a of the fluidic distribution block 650 (e.g., see “(B)” in FIG. 10H) having dimensions as described herein, and may have a lateral diameter at the distal face 99 of the proximal layer 650a of the fluidic distribution block 650 (e.g., see “(C)” in FIG. 10H) having dimensions as described herein. In some embodiments, the outlet apertures 302 may have a lateral diameter at the proximal face 98 of the distal layer 650b of the fluidic distribution block 650 (e.g., see “(D)” in FIG. 10H) having dimensions as described herein, and may have a lateral diameter at the distal face 97 of the distal layer 650b of the fluidic distribution block 650 (e.g., see “(E)” in FIG. 10H) having dimensions as described herein.

### II. Methods of Using a Microdose Device

In some embodiments, a method for using the fluid delivery device described herein (“microdose device”) is provided. In some embodiments, a method for delivering a fluidic composition across a dermal barrier of a patient is provided. In some embodiments, the method comprises: inserting a plurality of the microneedles of the microdose device described herein across the dermal barrier of the patient, and transporting the fluidic composition through the fluidic exit channels of the plurality of microneedles to a location below the dermal barrier.

In some embodiments, a method for delivering a fluidic composition across a dermal barrier of a patient is provided, the method comprising: penetrating the dermal barrier with a plurality of microneedles of the microdose device described herein, the microneedles optionally comprising a nanopatterned layer comprising nanostructures overlaid thereon, and transporting the fluidic composition through the fluidic exit channels of the plurality of microneedles to a location below the dermal barrier, wherein the number of microneedles in the plurality of microneedles is from 2 to 100 microneedles, such as up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 microneedles. In some embodiments, the microdose device may have one or more microneedles.

In some embodiments, the total volume of fluid delivered from the microdose device to a patient may be up to 2, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μL. In some embodiments, the total volume of fluid delivered from the microdose device to a patient may be over a time period of up to 1, 5, 10, 15, 20, 25, 30, 35, 40, 45, 50, 55, or 60 seconds, or up to 90, 120, 180, 210, 240, 270, or 300 seconds.

In some embodiments, a fluid volume from 1 μL to 500 μL may be delivered to a patient over a period of time from about 0.1 seconds to about 5 minutes, such as up to 1, 2, 3, 4, 5, 10, 15, 20, 25, 30, 35, 40, 45, 50, 55, or 60 seconds, or up to 90, 120, 180, 210, 240, 270, or 300 seconds. In some embodiments, the rate of fluid delivery per second per microneedle may be up to about 20 μL, 19 μL, 18 μL, 17 μL, 16 μL, 15 μL, 14 μL, 13 μL, 12 μL, 11 μL, 10 μL, 9 μL, 8 μL, 7 μL, 6 μL, 5 μL, 4 μL, 3 μL, 2 μL, 1 μL, 0.5 μL, 0.1 μL, or 0.01 μL, for example at a pressure of up to about 10 bar, 9 bar, 8 bar, 7 bar, 6 bar, 5 bar, 4 bar, 3 bar, 2 bar, 1 bar, 0.8 bar, 0.7 bar, 0.6 bar, 0.5 bar, 0.4 bar, 0.3 bar, 0.2 bar, or 0.1 bar. In some embodiments, the rate of delivery of the fluid from the microdose device to the patient may be constant or variable, such as an increasing rate, or a decreasing rate, or a pulsatile rate, or any combinations thereof.

In some embodiments, a fluid volume of up to 1 μL, 5 μL, 10 μL, 20 μL, 30 μL, 40 μL, 50 μL, 60 μL, 70 μL, 80 μL, 90 μL, 100 μL, 110 μL, 120 μL, 130 μL, 140 μL, 150 μL, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μL may be delivered to a patient over a period of time of less than 5 minutes, such as up to 1, 2, 3, 4, 5, 10, 15, 20, 25, 30, 35, 40, 45, 50, 55, or 60 seconds, or up to 90, 120, 180, 210, 240, 270, or 300 seconds.

In some embodiments, when the microdose device 10 of the present disclosure is coupled to a syringe barrel 210, following penetration of the dermal barrier of the patient by the plurality of microneedles, in response to an axial force applied to the plunger 220 in a distal direction, a fluid in the bore 213 of the barrel 210 of the syringe 20 flows from the syringe 20, through the microdose device 10 and exits the fluidic exit channels 155c of the plurality of microneedles 156 of the microdose device 10, thereby transporting the fluidic composition through the fluidic exit channels of the plurality of microneedles to a location below the dermal barrier of the patient.

In some embodiments, the method is performed by a medical professional, such as a physician or a nurse.

In some embodiments, the method further includes transporting the fluidic composition to the lymphatic system of the patient. In some embodiments, the method further includes transporting the fluidic composition to the blood circulatory system of the patient.

In use, when the microdose device of the present disclosure is coupled to a syringe for administering a fluid to a patient, in some embodiments, the plurality of microneedles may be placed on the skin of the patient proximate to a first position under the skin of the patient, wherein the first position is proximate to lymph vessels and/or lymph capillaries in the patient's lymphatic system. The plurality of microneedles may then be inserted the into the patient to a depth whereby at least the epidermis is penetrated and an end of at least one of the microneedles is proximate to the first position. A volume of the fluid may then be delivered from the microdose device via the plurality of microneedles to the first position in response to applying an axial force to the plunger of the syringe.

In some embodiments, the method includes placing the microdose device in direct contact with the skin of the patient. In some embodiments, an intervening layer or structure may be placed between the skin of the patient and the microdose device. For example, surgical tape or gauze may be used to reduce possible skin irritation between the microdose device and the skin of the patient. In some embodiments of the method, the microneedles will penetrate the epidermis or dermis of the patient in order to deliver the medicament to the patient. The delivery of the fluidic composition can be to the blood circulatory system, the lymphatic system, the interstitium, subcutaneous, intramuscular, intradermal or a combination thereof. In some embodiments, the fluidic composition is delivered directly to the lymphatic system of the patient. In some embodiments, the fluidic composition is delivered to the superficial vessels of the lymphatic system.

In some embodiments, the method includes placing the microdose device on an area of the patient's skin, in which a dense network of lymphatic capillaries and/or blood capillaries is present. Multiple microdose devices may be placed on one or more locations within the area, or the same microdose device may be sequentially placed on one or more locations within the area. In some embodiments, the method may include placing one or more e.g. 1, 2, 3, 4, 5, or more microdose devices on the subject's skin. These microdose devices may be placed spatially separate or in close proximity or juxtaposed with one another. The one or more microdose devices may be the same device sequentially placed onto the skin at the same or different locations, or may be different microdose devices placed onto the skin at the same or different locations.

In some embodiments, at least a portion of or all of the fluidic composition may be directly delivered or administered to an initial depth in the skin comprising the nonviable epidermis and/or the viable epidermis. In some embodiments, a portion of the fluidic composition may also be directly delivered to the viable dermis in addition to the epidermis. The range of delivery depth will depend on the medical condition being treated and the skin physiology of a given subject. This initial depth of delivery may be defined as a location within the skin, wherein a therapeutic agent first comes into contact as described herein. Without being bound by any theory, it is thought that the administered agent may move (e.g., diffuse) from the initial site of delivery (e.g., the non-viable epidermis, the viable epidermis, the viable dermis, or the interstitium) to a deeper position within the viable skin. For example, a portion of or all of an administered agent may be delivered to the non-viable epidermis and then continue to move (e.g., diffuse) into the viable epidermis and past the basal layer of the viable epidermis and enter into the viable dermis. Alternatively, a portion of or all of an administered agent may be delivered to the viable epidermis (i.e., immediately below the stratum corneum) and then continue to move (e.g., diffuse) past the basal layer of the viable epidermis and enter into the viable dermis. Lastly, a portion of or all of an administered agent may be delivered to the viable dermis. The movement of the one or more active agents throughout the skin is multifactorial and, for example, depends on the liquid carrier composition (e.g., viscosity thereof), rate of administration, delivery structures, etc. This movement through the epidermis and into the dermis may be further defined as a transport phenomenon and quantified by mass transfer rate(s) and/or fluid mechanics (e.g., mass flow rate(s)).

Thus, in some embodiments described herein, the agent may be delivered to a depth in the epidermis wherein the agent moves past the basal layer of the viable epidermis and into the viable dermis. In some embodiments, the agent is then absorbed by one or more susceptible lymphatic capillary plexus then delivered to one or more lymph nodes and/or lymph vessels.

Because the thickness of the skin can vary from patient to patient based on numerous factors, including, but not limited to, medical condition, diet, gender, age, body mass index, and body part, the depth below the skin surface to deliver the fluidic composition may vary. In some aspects, the delivery depth is from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some aspects, the delivery depth is about 50 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, or about 1000 μm. As used in this context, “about” means±50 μm.

In some embodiments described herein, the therapeutic agent is delivered to the viable skin, wherein the distribution of depths in the viable skin for delivery of the agent is immediately past the stratum corneum of the epidermis but above the subcutaneous tissue, which results in uptake of the agent by the lymphatic vasculature of the patient. In some aspects, the depth in the viable skin for delivering one or more agents ranges from about 1 μm to about 4,500 μm beyond the stratum corneum, but still within the viable skin above the subcutaneous tissue.

Non-limiting tests for assessing initial delivery depth in the skin may be invasive (e.g., a biopsy) or non-invasive (e.g., imaging). Conventional non-invasive optical methodologies may be used to assess delivery depth of an agent into the skin including remittance spectroscopy, fluorescence spectroscopy, photothermal spectroscopy, or optical coherence tomography (OCT). Imaging using methods may be conducted in real-time to assess the initial delivery depths. Alternatively, invasive skin biopsies may be taken immediately after administration of an agent, followed by standard histological and staining methodologies to determine delivery depth of an agent. For examples of optical imaging methods useful for determining skin penetration depth of administered agents, see, Sennhenn et al., Skin Pharmacol. 6(2) 152-160 (1993), Gotter et al., Skin Pharmacol. Physiol. 21 156-165 (2008), or Mogensen et al., Semin. Cutan. Med. Surg 28 196-202 (2009), each of which are incorporated by reference herein for their teachings thereof.

In some embodiments, the fluidic composition is delivered to the interstitium of the patient, e.g., to a space between the skin and one or more internal structures, such as an organ, muscle, or vessel (artery, vein, or lymph vessel), or any other spaces within or between tissues or parts of an organ. In some embodiments, the fluidic composition is delivered to both the interstitium and the lymphatic system.

In some embodiments, the method further comprises increasing permeability of the lymphatic vasculature wherein the nanostructures are in contact with, or proximate to, epithelial cells of the subject, thereby opening intercellular junctions between the epithelial cells and facilitating the flow of the fluidic composition during transport to the location below the dermal barrier.

In some embodiments described herein, the microdose device as described herein functions as a permeability enhancer and may increase the delivery of the fluidic composition through the epidermis. This delivery may occur through modulating transcellular transport mechanisms (e.g., active or passive mechanisms) or through paracellular permeation. Without being bound by any theory, the nanostructures of the nanopatterned layer may increase the permeability of one or more layers of the viable epidermis, including the epidermal basement membrane by modifying cell/cell tight junctions allowing for paracellular or modifying cellular active transport pathways (e.g., transcellular transport) allowing for diffusion or movement and/or active transport of an administered agent through the viable epidermis and into the underlying viable dermis. This effect may be due to modulation of gene expression of the cell/cell tight junction proteins. As previously mentioned, tight junctions are found within the viable skin and in particular the viable epidermis. The opening of the tight junctions may provide a paracellular route for improved delivery of any agent, such as those that have previously been blocked from delivery through the skin.

Interaction between individual cells and structures of the nanotopography may increase the permeability of an epithelial tissue (e.g., the epidermis) and induce the passage of an agent through a barrier cell and encourage transcellular transport. For instance, interaction with keratinocytes of the viable epidermis may encourage the partitioning of an agent into the keratinocytes (e.g., transcellular transport), followed by diffusion through the cells and across the lipid bilayer again. In addition, interaction of the nanotopography structure and the corneocytes of the stratum corneum may induce changes within the barrier lipids or corneodesmosomes resulting in diffusion of the agent through the stratum corneum into the underlying viable epidermal layers. While an agent may cross a barrier according to paracellular and transcellular routes, the predominant transport path may vary depending upon the nature of the agent.

In some embodiments, the microdose device may interact with one or more components of the epithelial tissue to increase porosity of the tissue making it susceptible to paracellular and/or transcellular transport mechanisms. Epithelial tissue is one of the primary tissue types of the body. Epithelial tissues that may be rendered more porous may include both simple and stratified epithelium, including both keratinized epithelium and transitional epithelium. In addition, epithelial tissue encompassed herein may include any cell types of an epithelial layer including, without limitation, keratinocytes, endothelial cells, lymphatic endothelial cells, squamous cells, columnar cells, cuboidal cells and pseudostratified cells. Any method for measuring porosity may be used including, but not limited to, any epithelial permeability assay. For example, a whole mount permeability assay may be used to measure epithelial (e.g., skin) porosity or barrier function in vivo see, for example, Indra and Leid., Methods Mol Biol. (763) 73-81, which is incorporated by reference herein for its teachings thereof.

In some embodiments, the structural changes induced by the presence of a nanotopography (the nanopatterned layer having a plurality of nanostructures) on a barrier cell are temporary and reversible, including reversible increase in the porosity of epithelial tissues by changing junctional stability and dynamics, which, without being bound by any theory, may result in a temporary increase in the paracellular and transcellular transport of an administered agent through the epidermis and into the viable dermis. Thus, in some aspects, the increase in permeability of the epidermis or an epithelial tissue elicited by the nanotopography, such as promotion of paracellular or transcellular diffusion or movement of one or more agents, returns to a normal physiological state that was present before contacting the epithelial tissue with a nanotopography following the removal of the nanotopography. In this way, the normal barrier function of the barrier cell(s) (e.g., epidermal cell(s)) is restored and no further diffusion or movement of molecules occurs beyond the normal physiological diffusion or movement of molecules within the tissue of a patient.

These reversible structural changes induced by the nanotopography may function to limit secondary skin infections, absorption of harmful toxins, and limit irritation of the dermis. Also, the progressive reversal of epidermal permeability from the top layer of the epidermis to the basal layer may promote the downward movement of one or more agents through the epidermis and into the dermis and prevent back flow or back diffusion of the one or more agents back into the epidermis.

In some embodiments, a method for administering a fluidic composition to the lymphatic system of a patient is provided, comprising applying the microdose device described herein to deliver the fluidic composition to the lymphatic system. Delivery to the lymphatic system encompasses, e.g., delivery to a target in the lymphatic system or delivery through the lymphatic system to the systemic circulation or to a non-lymphatic target, for example which may include without limitation a circulating cells, an organ, a tissue, and so on.

The fluidic composition may comprise one or more agents to be delivered to a therapeutic target. In some embodiments, the therapeutic target is a lymph node, a lymph vessel, an organ that is part of the lymphatic system or a combination thereof. In some embodiments, the therapeutic target is a lymph node. In some embodiments, the therapeutic target is a specific lymph node as described elsewhere herein.

In some embodiments, delivery of the therapeutic agent to the lymphatic system is delivery into the vessels of the lymphatic vasculature, the lymph nodes as described elsewhere herein, or both. In some embodiments, delivery is to the superficial lymph vessels. In yet another aspect, delivery is to one or more lymph nodes.

Because lymph nodes often occur in a group as opposed to being present as a single isolated node, the term “lymph node” as used herein can be singular or plural and refer to either a single isolated lymph node or a group of lymph nodes in a small physical location. For example, a reference to the inguinal lymph node or inguinal lymph nodes refers to the group of lymph nodes that are recognized by a person skilled in the art as a group of lymph nodes located in the hip/groin area or femoral triangle in a patient. It also refers to both the superficial and deep lymph nodes unless specifically stated otherwise.

In some embodiments, the lymph node is selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, the medicament is delivered to the interstitium of the patient, e.g., to a space between the skin and one or more internal structures, such as an organ, muscle, or vessel (artery, vein, or lymph vessel), or any other spaces within or between tissues or parts of an organ. In still yet another embodiment, the medicament is delivered to both the interstitium and the lymphatic system. In embodiments where the therapeutic agent is delivered to the interstitium of the patient, it may not be necessary to locate the lymph nodes or lymphatic vasculature of the patient before administering the therapeutic agent.

In some embodiments, disclosed herein is a method for administering a therapeutic agent to the lymphatic system of a patient. The method generally comprises placing a microdose device described herein comprising a plurality of microneedles on the skin of the patient at a first location proximate to a first position under the skin of the patient, wherein the first position is proximate to lymph vessels and/or lymph capillaries, optionally wherein the microneedles of the microdose device have a surface comprising nanotopography, inserting the plurality of microneedles of the microdose device into the patient to a depth whereby at least the epidermis is penetrated and an end of at least one of the protrusions is proximate to the first position, and administering via the microneedles of the microdose device a dose of the therapeutic agent into the first position.

In some embodiments, a dose of the therapeutic agent may be a therapeutically effective amount. In some embodiments, one dose of the therapeutic agent may not be a therapeutically effective amount, and so more than one dose may be administered to the patient. In some embodiments, the combined amount of the doses is therapeutically effective. In some embodiments, at least two doses can be administered to the patient. The at least two doses can be administered sequentially or simultaneously.

The one or more doses that are therapeutically effective may be smaller doses than a therapeutically effective dose if the agent is administered by a different route (e.g., intravenous, subcutaneous, intramuscular, intradermal or parenteral delivery routes, etc.).

In some embodiment, disclosed herein is a method for increasing the bioavailability of a therapeutic agent in a patient, the method comprising placing at least one microdose device described herein on the skin surface of the subject; and administering a therapeutic agent with the at least one microdose device to the subject.

In some embodiments, the method of delivering a therapeutic agent to a patient as described herein may result in an equivalent blood serum absorption rate of a therapeutic agent as compared to intravenous, subcutaneous, intramuscular, intradermal or parenteral delivery routes while retaining relatively higher rates of lymphatic delivery as described herein. Without being bound by any theory, the rate of delivery and increased bioavailability may be due to the lymphatic circulation of one or more agents through the thoracic duct or the right lymphatic duct and into the blood circulation. Standard highly accurate and precise methodologies for measuring blood serum concentration and therapeutic monitoring at desired time points may be used that are well known in the art, such as radioimmunoassays, high-performance liquid chromatography (HPLC), fluorescence polarization immunoassay (FPIA), enzyme immunoassay (EMIT) or enzyme-linked immunosorbant assays (ELISA). For calculating the absorption rate using the methods described above, the drug concentration at several time points may be measured starting immediately following administration and incrementally thereafter until a Cₘₐₓ value is established and the associated absorption rate calculated.

The terms “medicament”, “medication”, “medicine”, “therapeutic agent” and “drug” are used interchangeably herein and describe a pharmaceutical composition or product intended for the treatment of a medical condition having at least one symptom. The pharmaceutical composition or product will have a physiological effect on the patient when it is introduced into the body of a patient. The pharmaceutical composition can be in any suitable formulation unless a specific formulation type is required or disclosed. In some instances, the medicament will be approved by the US FDA while in other instances it may be experimental (e.g., in clinical or pre-clinical trials) or approved for use in a country other than the United States (e.g., approved for use in China or Europe). In instances where these terms are used, it is understood that they refer to both singular and plural instances. In some embodiments herein, two or more medicaments may be used in a form of combination therapy. In all cases, the selection of the proper medicament (singular or plural) will be based on the medical condition of the patient and the assessment of the medical professional administering, supervising and/or directing the treatment of the patient. Combination therapies are sometimes more effective than a single agent and used for many different medical conditions. It is understood that combination therapies are encompassed herein and envisioned with the subject matter disclosed.

An “effective amount” or a “therapeutically effective dose” in reference to a medicament is an amount sufficient to treat, ameliorate, or reduce the intensity of at least one symptom associated with the medical condition. In some aspects of this disclosure, an effective amount of a medicament is an amount sufficient to effect a beneficial or desired clinical result including alleviation or reduction in one or more symptoms of a medical condition. In some embodiments, an effective amount of the medicament is an amount sufficient to alleviate all symptoms of a medical condition. In some aspects, a dose of the therapeutic agent will be administered that is not therapeutically effective by itself. In these aspects, multiple doses may be administered to the patient either sequentially (using the same microdose device or different microdose devices) or simultaneously such that the combination of the individual doses is therapeutically effective. For simultaneous administration, additional medical microdose devices comprising a plurality of protrusions or an entirely different route of administration may be used.

The term “patient” as used herein refers to a warm blooded animal such as a mammal which is the subject of a medical treatment for a medical condition that causes at least one symptom. It is understood that at least humans, dogs, cats, and horses are within the scope of the meaning of the term. In some embodiments, the patient is a human.

As used herein, the term “treat” or “treatment”, or a derivative thereof, contemplates partial or complete amelioration of at least one symptom associated with the medical condition of the patient, including but not limited to slowing or arresting the worsening of a symptom that would occur in the absence of treatment. “Preventing” a symptom or medical condition from occurring is considered a form of treatment. “Reducing” the incidence of a symptom or medical condition is considered a form of treatment.

As used herein, “bioavailability” means the total amount of a given dosage of the administered agent that reaches the blood compartment measured as a ratio of (AUC/dose) for a given route of administration/(AUC/dose) for intravenous administration with the area under the curve (AUC) in a plot of concentration vs. time.

Cₘₐₓ refers to the maximum concentration that a medicament achieves in the plasma or tissue of a patient after the medicament has been administered while Ct refers to the concentration that a medicament achieves at a specific time (t) following administration. Unless otherwise stated, all discussion herein is in regard to pharmacokinetic parameters in plasma.

The AUCₜ refers to the area under the plasma concentration time curve from time zero to time t following administration of the medicament.

The AUC_{∞} refers to the area under the plasma concentration time curve from time zero to infinity (infinity meaning that the plasma concentration of the medicament is below detectable levels).

Tₘₐₓ is the time required for the concentration of a medicament to reach its maximum blood plasma concentration in a patient following administration. Some forms of administration of a medicament will reach their Tₘₐₓ slowly (e.g., tablets and capsules taken orally) while other forms of administration will reach their Tₘₐₓ almost immediately (e.g., subcutaneous and intravenous administration).

“Steady state” refers to the situation where the overall intake of a drug is approximately in dynamic equilibrium with its elimination.

A discussion of various pharmacokinetic parameters and the methods of measuring and calculating them can be found in Clinical Pharmacokinetics and Pharmacodynamics: Concepts and Applications, M. Rowland and T. N. Tozer, (Lippincott, Williams & Wilkins, 2010) which is incorporated by reference for its teachings thereof.

The lymphatic system plays an important role in transporting body fluids and particulate materials throughout the body. The lymphatic system comprises several lymph organs (e.g., the spleen and thymus) in addition to lymph nodes, lymph vessels and lymph capillaries. The vessels transport lymph fluid around the body in a single direction in either the superficial vessels or the deep vessels (i.e., the lymphatic vasculature). Drainage begins in blind capillaries which gradually develop into vessels. These vessels then travel through several lymph nodes. The lymph nodes contain both T and B lymphocytes in addition to other cells associated with the immune system. Antigens and other foreign particles are filtered out in the lymph nodes. The lymph vessels eventually end in either the right lymphatic duct which drains into the right internal jugular vein or the thoracic duct which drains into the subclavian vein. It is a one-way system where the lymph fluid (also referred to a lymph) is eventually returned to the circulatory system of the patient.

In some embodiments described herein, the therapeutic agent may be delivered in a liquid carrier solution. In one aspect, the tonicity of the liquid carrier may be hypertonic to the fluids within the blood capillaries or lymphatic capillaries. In another aspect, the tonicity of a liquid carrier solution may be hypotonic to the fluids within the blood capillaries or lymphatic capillaries. In another aspect, the tonicity of a liquid carrier solution may be isotonic to the fluids within the blood capillaries or lymphatic capillaries. The liquid carrier solution may further comprise at least one or more pharmaceutically acceptable excipients, diluent, cosolvent, particulates, or colloids. Pharmaceutically acceptable excipients for use in liquid carrier solutions are known, see, for example, Pharmaceutics: Basic Principles and Application to Pharmacy Practice (Alekha Dash et al. eds., 1st ed. 2013), which is incorporated by reference herein for its teachings thereof.

In some embodiments described herein, the therapeutic agent is present in a liquid carrier as a substantially dissolved solution, a suspension, or a colloidal suspension. Any suitable liquid carrier solution may be utilized that meets at least the United States Pharmacopeia (USP) specifications, and the tonicity of such solutions may be modified as is known, see, for example, Remington: The Science and Practice of Pharmacy (Lloyd V. Allen Jr. ed., 22nd ed. 2012. Exemplary non-limiting liquid carrier solutions may be aqueous, semi-aqueous, or nonaqueous depending on the bioactive agent(s) being administered. For example, an aqueous liquid carrier may comprise water and any one of or a combination of a water-miscible vehicles, ethyl alcohol, liquid (low molecular weight) polyethylene glycol, and the like. Non-aqueous carriers may comprise a fixed oil, such as corn oil, cottonseed oil, peanut oil, or sesame oil, and the like. Suitable liquid carrier solutions may further comprise any one of a preservative, antioxidant, complexation enhancing agent, a buffering agent, an acidifying agent, saline, an electrolyte, a viscosity enhancing agent, a viscosity reducing agent, an alkalizing agent, an antimicrobial agent, an antifungal agent, a solubility enhancing agent or a combination thereof.

### III. Methods of Administering a Coronavirus Vaccine

In some embodiments, the fluidic composition may comprise a coronavirus vaccine. In particular, in some embodiments, the fluidic composition comprises a SARS-CoV-2 vaccine.

More than 180 vaccine candidates, based on several different platforms, are currently in development against SARS-CoV-2 (for a review, see e.g., Krammer, F. SARS-CoV-2 vaccines in development. Nature 586, 516-527 (2020). https://doi.org/10.1038/s41586-020-2798-3).

The World Health Organization (WHO) maintains a working document that includes most of the vaccines in development and is available at https://www.who.int/publications/m/item/draft-landscape-of-covid-19-candidate-vaccines. The current title of the document is Draft Landscape of COVID-19 Candidate Vaccines. https://www.who.int/publications/rn/iterm/draft-landscape-of-covid-19-candidate-vaccines (WHO, accessed Nov. 12, 2020) (also referred to herein as “DRAFT landscape of COVID-19 candidate vaccines”).

The vaccine platforms can be divided into ‘traditional’ approaches (inactivated or live-virus vaccines), recombinant protein vaccines and vectored vaccines, and nucleic acid (RNA and DNA) vaccines.

Inactivated vaccines are typically produced by growing SARS-CoV-2 in cell culture, e.g. on Vero cells, followed by chemical inactivation of the virus.

Live attenuated vaccines are typically produced by generating a genetically weakened version of the virus that replicates to a limited extent, causing no disease but inducing immune responses that are similar to that induced by natural infection. Attenuation can be achieved for example by adapting the virus to unfavorable conditions (for example, growth at lower temperature, growth in non-human cells) or by rational genetic modification of the virus (for example, by codon de-optimization or by deleting genes that are responsible for counteracting innate immune recognition).

Recombinant protein vaccines can be divided into recombinant spike-protein-based vaccines, recombinant RBD-based vaccines, and virus-like particle (VLP)-based vaccines. These recombinant proteins can be expressed in different expression systems including insect cells, mammalian cells, yeast, bacteria, and plants. Yields, and the type and extent of post-translational modifications, vary depending on the expression system. For recombinant spike-protein-based vaccines in particular, modifications such as deletion of the polybasic cleavage site, inclusion of stabilizing mutations, and inclusion of trimerization domains as well as the mode of purification (soluble protein versus membrane extraction) may vary.

Replication-incompetent vector vaccines are typically based on another virus that has been engineered to express the spike protein and has been disabled from replication in vivo by the deletion of parts of its genome. The majority of these approaches are based on adenovirus (AdV) vectors, although modified vaccinia Ankara (MVA), human parainfluenza virus vectors, influenza virus, adeno-associated virus and Sendai virus are used as well.

Replication-competent vectors are typically derived from attenuated or vaccine strains of viruses that have been engineered to express a transgene, e.g. the spike protein. For example, engineered influenza virus, measles virus, vesicular stomatitis virus (VSV), horsepox and Newcastle disease virus (NDV) may be used. In some cases, animal viruses that do not replicate efficiently and cause no disease in humans are used.

Some SARS-CoV-2 vaccine candidates that are currently under development use viral vectors that display the spike protein on their surface and are then inactivated before use. Examples of inactivated virus vectors include NDV-based vaccines that display the spike protein on their surface-which can be produced in a similar manner to influenza virus vaccines—as well as rabies vectors.

DNA vaccines are typically based on plasmid DNA that can be produced at large scale in bacteria. Typically, these plasmids contain mammalian expression promoters and the gene that encodes the spike protein, which is expressed in the patient upon delivery.

RNA vaccines are a relatively recent development. Similar to DNA vaccines, the genetic information for the antigen is delivered and the antigen is then expressed in the cells of the patient. Either mRNA or a self-replicating RNA can be used.

In some embodiments, a coronavirus vaccine of the present disclosure includes without limitation any of the vaccine candidates referred to in the DRAFT landscape of COVID-19 candidate vaccines, or otherwise identifiable by skilled persons upon reading the present disclosure.

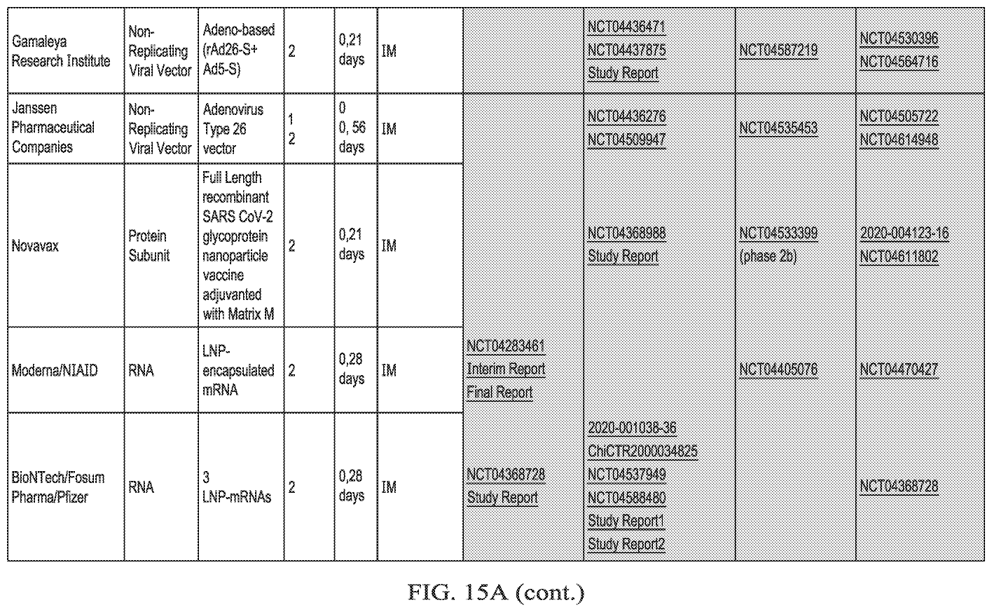

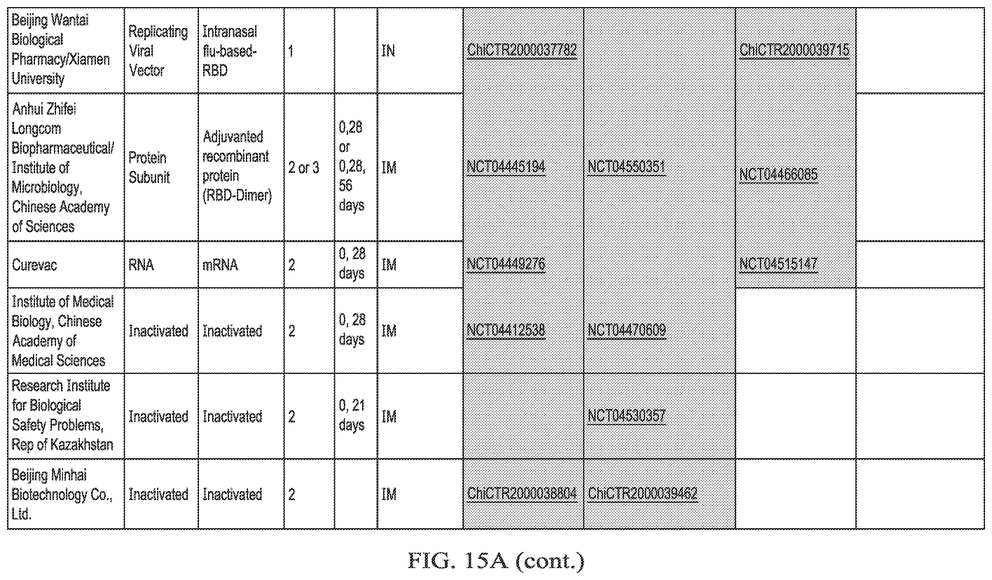

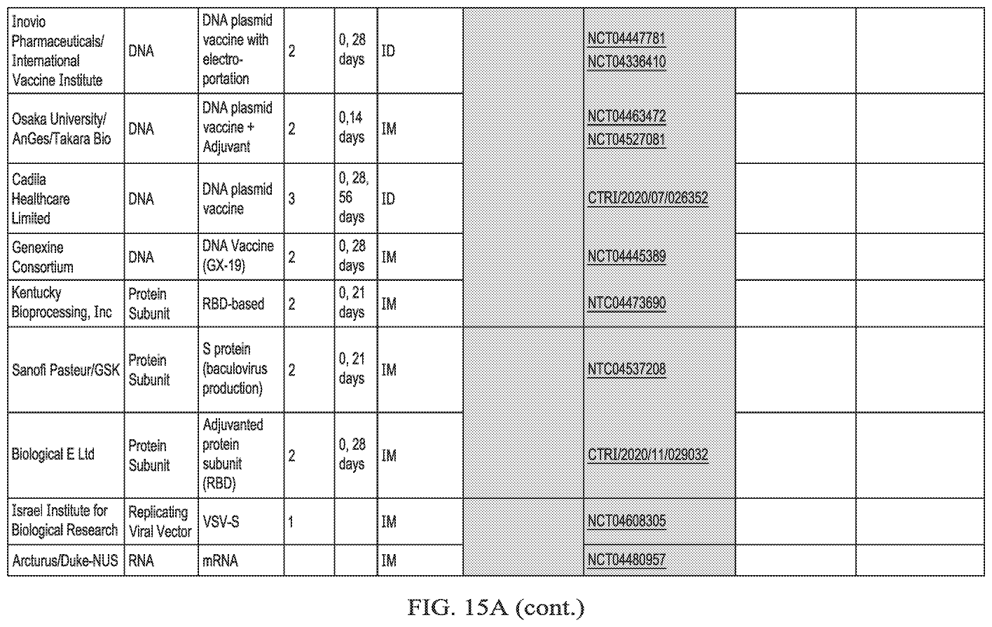

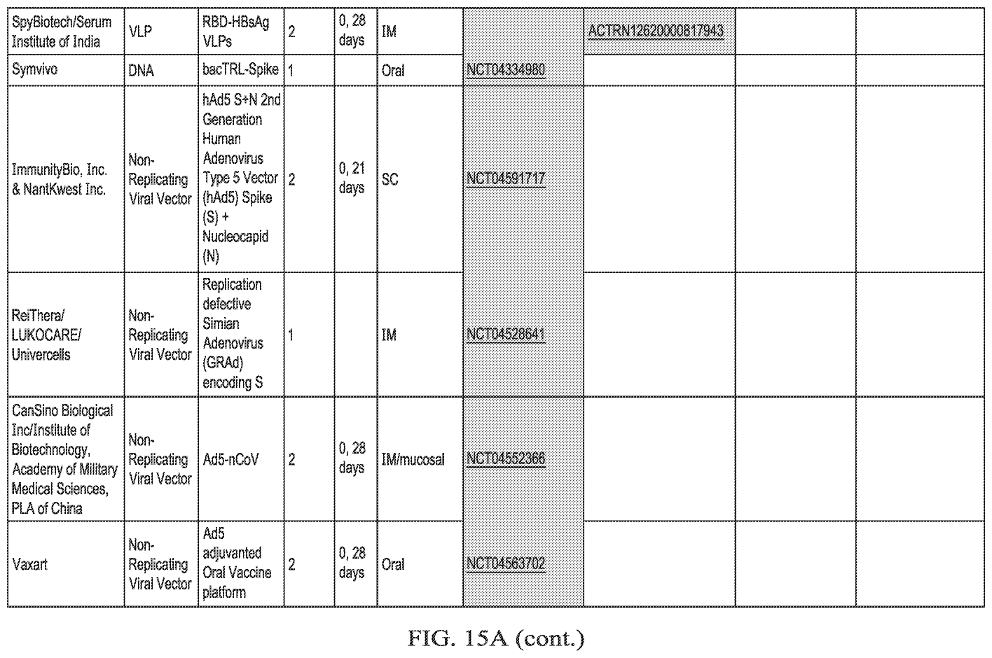

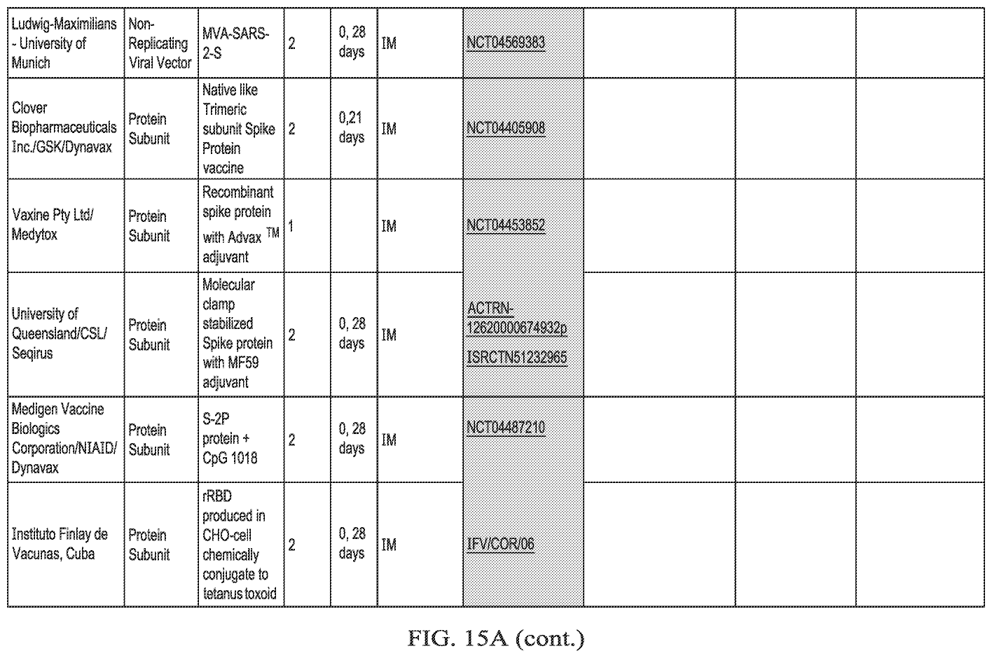

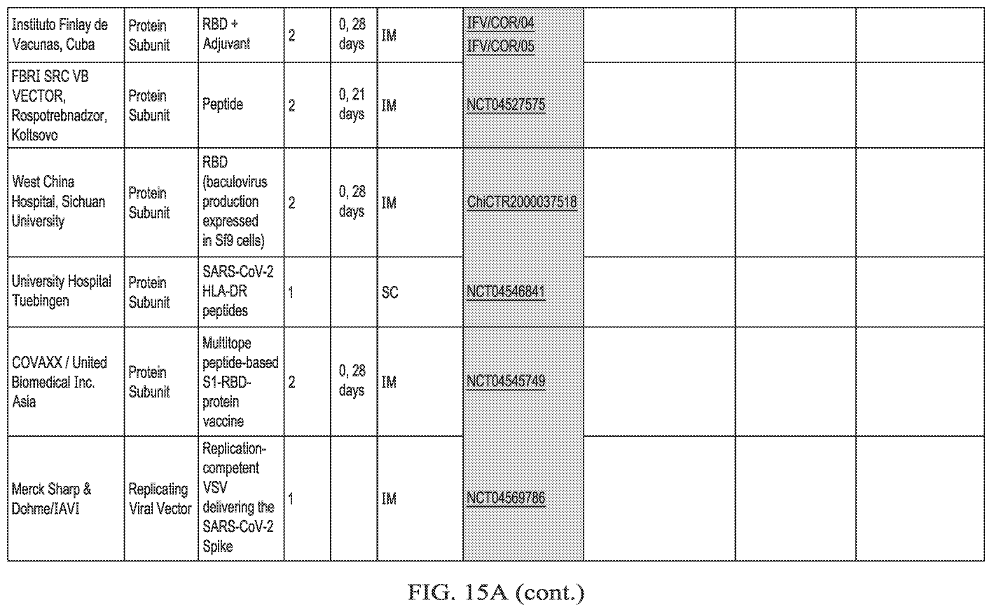

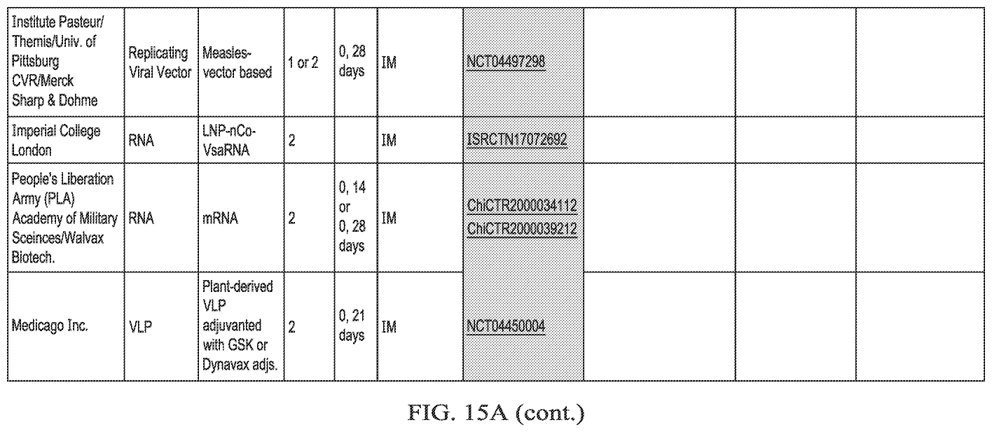

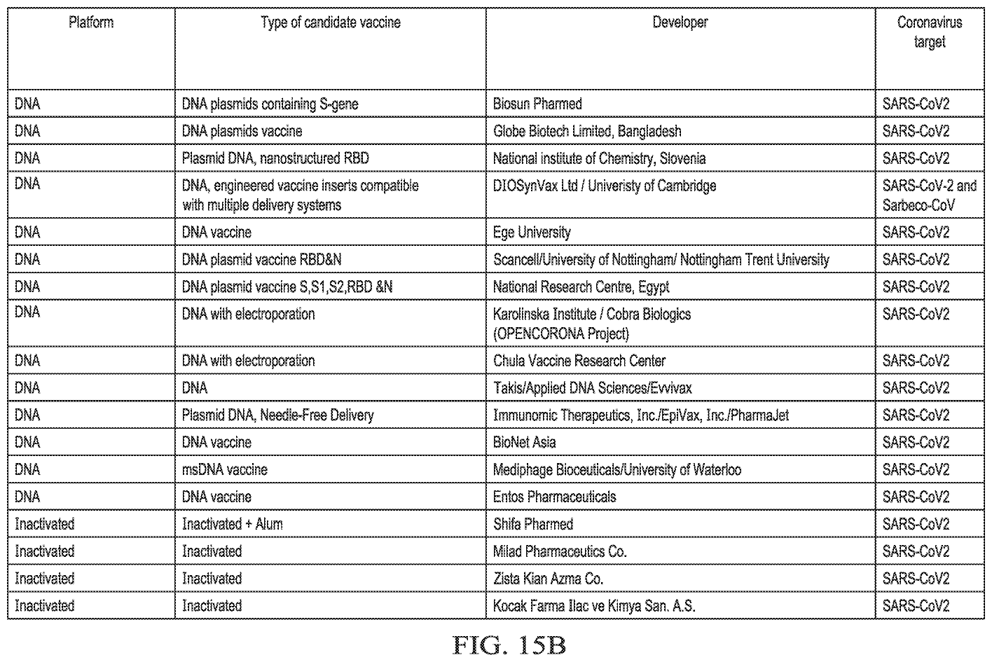

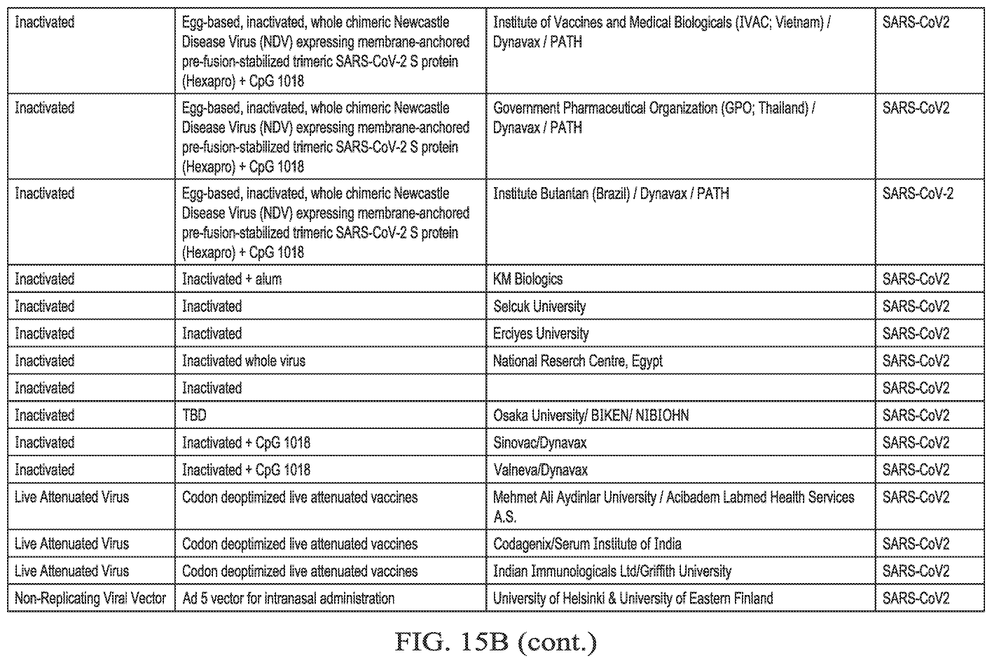

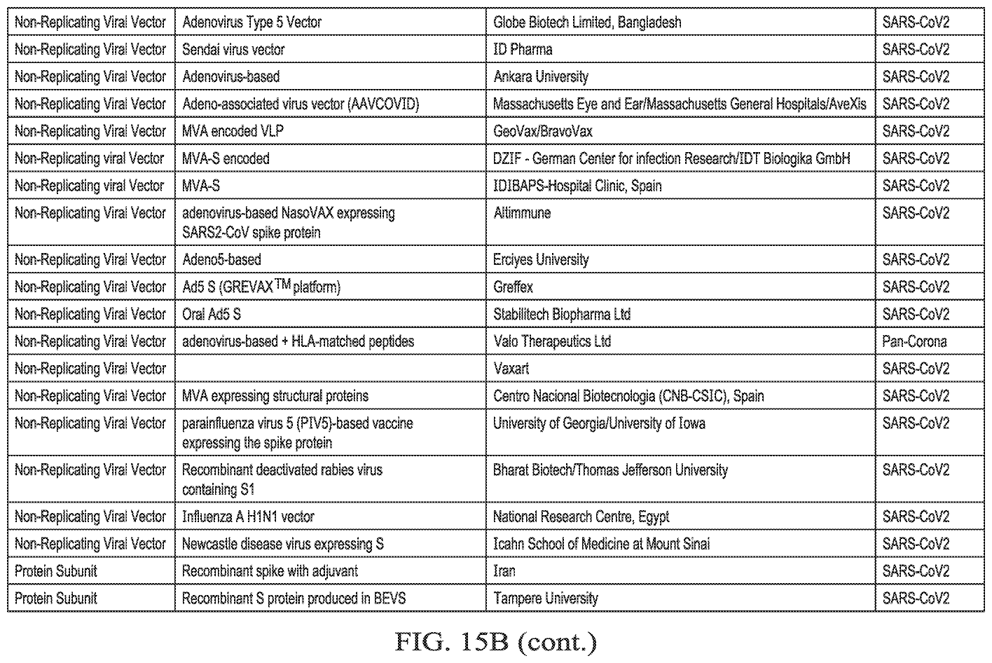

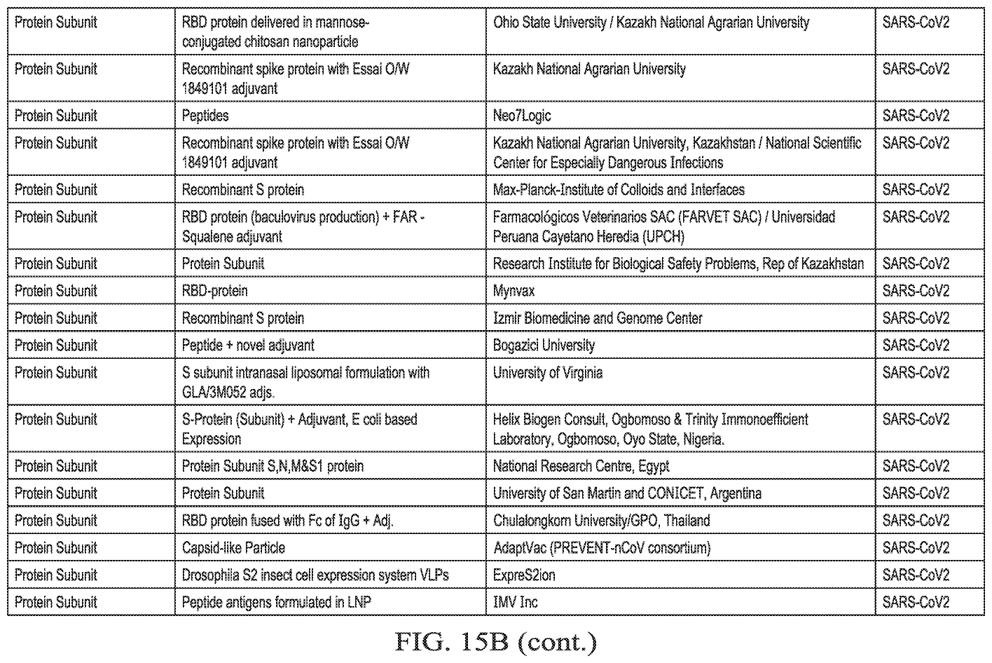

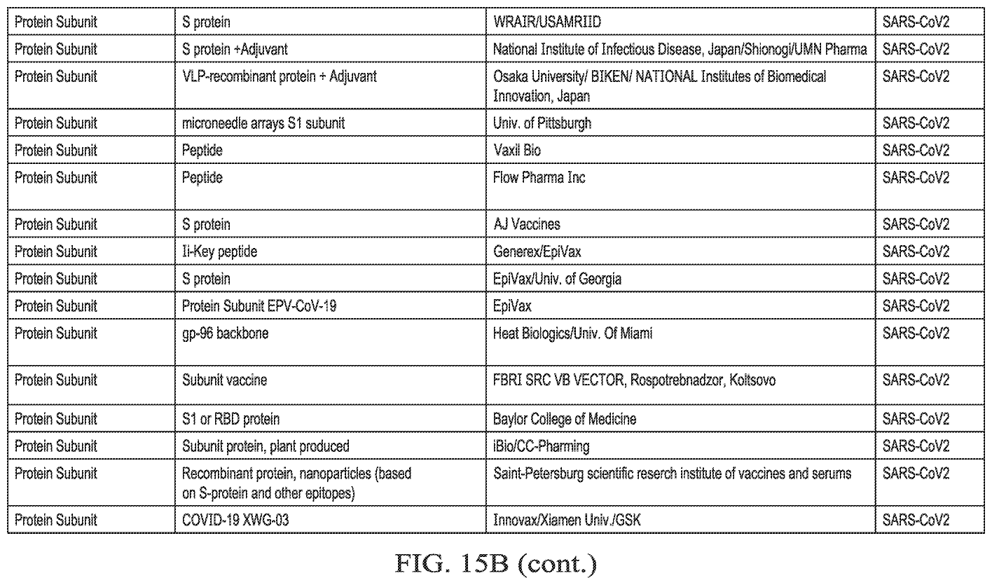

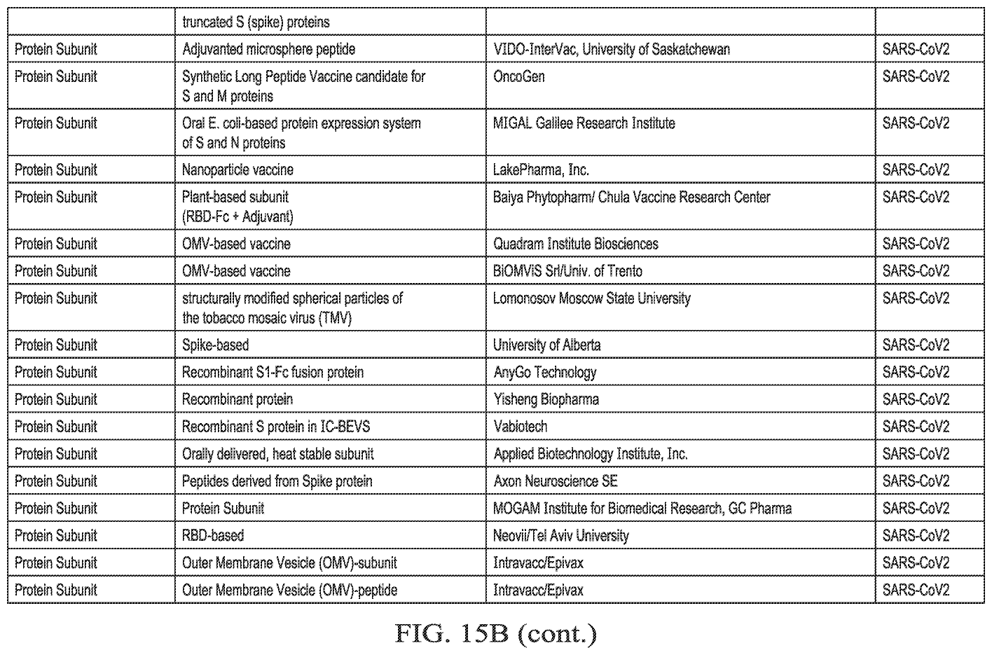

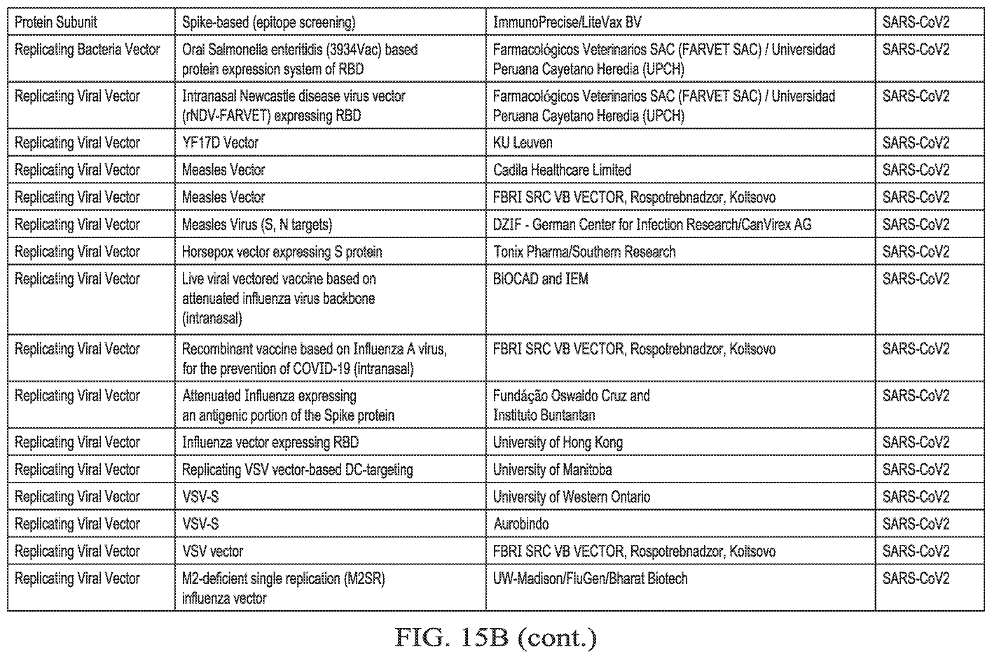

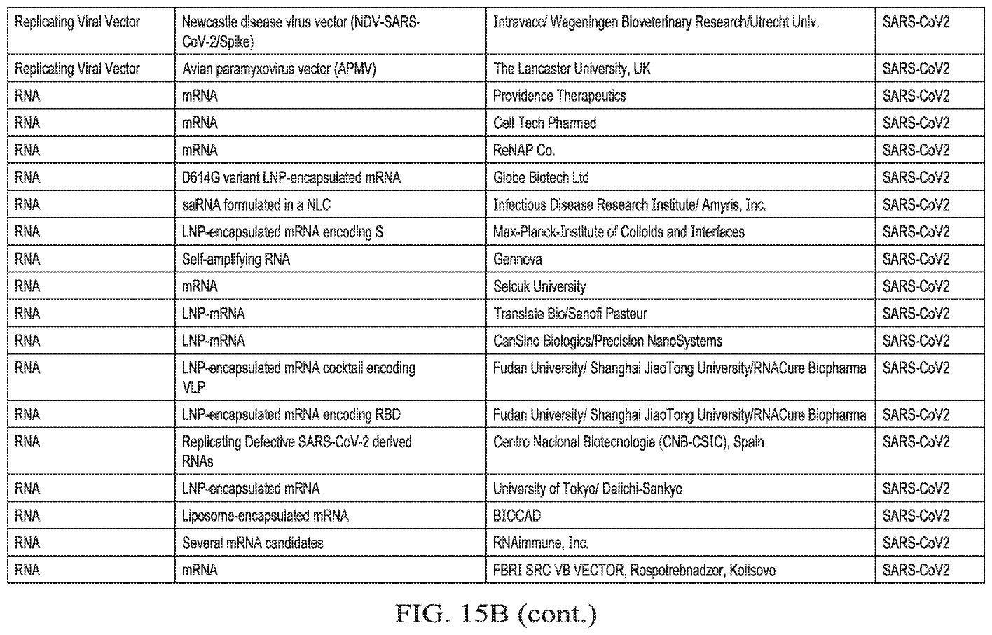

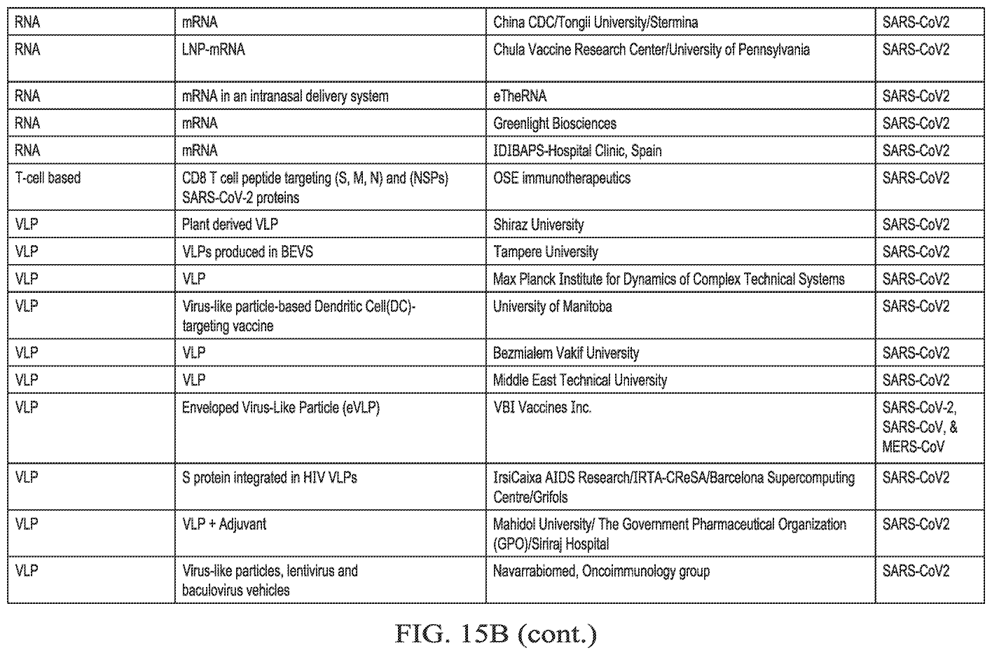

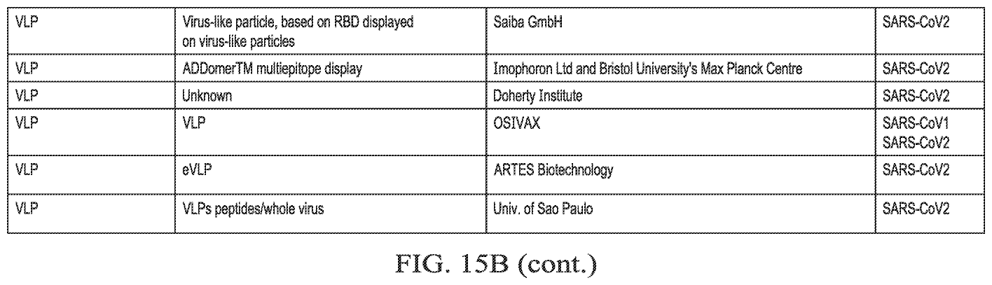

For example, a coronavirus vaccine of the present disclosure includes without limitation a vaccine candidate currently under clinical evaluation listed in the Table in FIG. 15A, or a vaccine candidate currently under preclinical evaluation listed in the Table in FIG. 151B (both Tables are from the WHO's draft landscape of COVID-19 candidate vaccines, accessed Nov. 12, 2020). Details listed in the Table of FIG. 15A include the vaccine developer/manufacturer, vaccine platform, type of candidate vaccine, the number of doses given to the patient in clinical trials, the timing of doses in clinical trials, the route of administration used in the clinical trials, and the clinical stage (phase 1, 1/2, 2, or 3, with clinical trial identifiers provided). Listed in the Table of FIG. 15B are the vaccine platform, the type of candidate vaccine, the developer, and the coronavirus target.

In some embodiments, the coronavirus vaccine may include without limitation a recombinant fusion protein comprising a coronavirus spike S1 protein or a fragment thereof linked to an immunoglobulin Fc region or a fragment thereof (also referred to herein as “rS1-Fc”), and nucleic acids (DNA or mRNA) encoding the rS1-Fc fusion proteins, and expression vectors comprising the nucleic acids, and compositions of any thereof, such as those described in U.S. Provisional Application No. 62/993,527, filed Mar. 23, 2020 and U.S. Provisional Application No. 63/045,685 Filed Jun. 29, 2020, and also described in Herrmann A, Maruyama J, Yue C, et al. A Targeted Vaccine against COVID-19: S1-Fc Vaccine Targeting the Antigen-Presenting Cell Compartment Elicits Protection against SARS-CoV-2 Infection. bioRxiv; 2020. DOI: 10.1101/2020.06.29.178616, the disclosures of which are incorporated herein in their entireties.

In some embodiments, the coronavirus spike S1 protein or a fragment thereof may be derived from SARS-CoV-2. In some embodiments, for example, as described in U.S. Provisional Application No.'s 62/993,527 and 63/045,685, the rS1-Fc vaccines described therein are expected to elicit a post-immunization response to SARS-CoV-2 in a patient administered with the rS1-Fc vaccine. In some embodiments, MHC Class I/II antigen presentation (e.g., presentation of a SARS-CoV-2 spike S1 protein fragment of the rS1-Fc fusion protein) by dendritic cells in the patient, generates both cytotoxic CD8-positive Tc cell responses and helper Th CD4-positive responses. As a result, CD4-positive T-cells are expected to activate B-cells to produce neutralizing antibodies against the SARS-CoV-2 spike S1 protein, and CD8-positive cytotoxic T-cells kill cells infected with SARS-CoV-2.

In some embodiments, rS1-Fc immunization elicits early seroconversion, facilitating anti-S1-specific IgG production protecting against live SARS-CoV-2 challenge. For example as described in U.S. Provisional Application No. 63/045,685, mice immunized by intramuscular injection with linearized dsDNA encoding S1-Fc mounted a significant and robust CD4⁺IFNγ⁺ Th1 polarization in vivo in a dose-dependent manner. Moreover, S1-antigen specific CD8⁺ T cells isolated from spleen accumulated upon immunization at increased dose. Furthermore, high dose immunization favored CD8⁺IFNγ⁺ effector T cell in vivo education in a dose-dependent manner. Thus, dose-dependent adaptive immune responses upon administration of S1-Fc dsDNA indicate that considerably elevated dosing with S1-Fc dsDNA, which is expected to continuously produce and systemically release the S1 antigen, is required to elicit a desired adaptive T cells immune response. Complete seroconversion was detectable at day 10 upon initial immunization with both 50 μg and 20 μg of administered S1-Fc dsDNA, mounting similar levels of S1-specific serum IgG antibodies. In addition, mice immunized by intramuscular injection with recombinant rS1-Fc protein facilitated accelerated seroconversion detectable at day 7, with considerable increases in levels of S1-specific serum IgG antibodies over time. Murine blood serum seropositive for anti-S1 IgG significantly reduced the interaction of the viral S i-domain and host receptor ACE2. Collected blood serum seropositive for anti-S1 IgG elicited protection against live SARS-CoV-2 infection in a stringent experimental virus challenge assay. Routing rS1-Fc administration via intravenous injection resulted in a similar protection efficacy.

The immunogenicity of a coronavirus vaccine may be assessed in preclinical studies, e.g. in mice, e.g. in C57BL/6 mice and Balb/c mice. Th1, Th2, Th17, and T-reg cytokine patterns may be evaluated e.g. using ELISA methods, flow cytometry methods, and other methods known in the art to observe whether the coronavirus vaccine will induce a more effective Th1 immune response (e.g., IFN-γ, and IL-12) with an absent, low or very low increase in IL-17 and IL-4 and an absent, low or very low increase in TGF-β.

In some embodiments, a coronavirus vaccine (e.g. an rS1-Fc vaccine, or some others described herein) may allow selective uptake into APCs, induce cross-presentation of coronavirus antigen proteins (e.g. spike S1 protein) or fragments thereof and elicit a robust anti-SARS-CoV-2 response in context of Th1/Th2 and Th17/T-reg balances, which may allow an immune response in a patient providing effective vaccination of a patient, treatment of a coronavirus infection in a patient and minimization or prevention of adverse immune-related effects in the patient.

In previous studies, efforts to develop respiratory virus vaccines to protect against Respiratory Syncytial Virus (RSV) and SARS-related disease have demonstrated the potential clinical benefits of eliciting a Th1 adaptive immune response over the disease-exacerbating effects of a Th2 polarized response. Immunization studies in mice with four candidate SARS vaccines (VLP, whole virus, and an rDNA-produced Spike protein) led to pulmonary immunopathology upon challenge with SARS virus, an effect that was signified by Th2 polarization in mice immunized with each candidate vaccine. In other previous studies, use of APC-engaging antigens in the development of tumor vaccines, such as fusing the ectodomain of the XCL1 ligand with XCR1 receptors on the surface of DCs, has been shown to engage dendritic cells in the periphery of immunized mice and elicit a predominantly Th1-polarized response. Direct engagement of DCs in this manner leads to cross presentation of tumor antigens, expression of inflammatory cytokines including IL-12 and IFNγ, recruitment of NK cells, and emergence of a Th1 cytotoxic T cell response.

In some embodiments, it is expected that administration of the rS1-Fc vaccine described herein may result in an increased Th1 polarization response and low or absent mixed Th1/Th2 or predominantly Th2 responses.

In some embodiments, it is expected that the rS1-Fc vaccine described herein may enhance APC-specific targeting and enhance Th1 immunization and prevent tolerance induction.

In some embodiments, administering a coronavirus vaccine, e.g. an rS1-Fc vaccine, or other coronavirus vaccines described herein, to a patient delivered using the microdose devices and methods as described herein may provide one or more therapeutic advantages as compared to delivering a coronavirus vaccine to a patient using other routes of administration such as intramuscular, intravenous, or intradermal delivery.

Without limitation to theory, in some embodiments, the microdose devices and methods of the present disclosure are configured to deliver a fluid composition comprising a therapeutic agent (e.g. a coronavirus vaccine) just above the epidermal ridge/dermal papillae and access the apical dermis which contains 10-fold more dendritic cells than the entire blood volume of an average individual (X-N Wang et al., A Three-Dimensional Atlas of Human Dermal Leukocytes, Lymphatics, and Blood Vessels. Investigative Society of Investigative Dermatology, 2014. Volume 134, Issue 4, Pages 965-974. doi.org/10.1038/jid.2013.481). Without limitation to theory, dermal dendritic cells (but not macrophages) migrate into lymphatic vessels which is expected to improve immunity.

In some embodiments, the microdose device of the present disclosure may deliver from about 10 to 40 times higher (e.g., about 15 times higher) therapeutic agent (e.g. a coronavirus vaccine) concentrations to lymph nodes as compared to other routes of administration such as intravenous, subcutaneous, intramuscular, or intradermal injections.

In some embodiments, it is expected that lymphatic delivery of a therapeutic agent (e.g. a coronavirus vaccine) using the microdose device of the present disclosure may provide improvements in immunity against a coronavirus infection, including increases in coronavirus antigen-specific IgG levels and/or T-cell responses, as compared to other routes of administration such as such as intravenous, subcutaneous, intramuscular, or intradermal injections. Because the microdose device described herein is adapted to deliver fluidic compositions to the lymphatic system of a patient (e.g. see Example 1), similar improvements in immunity against a coronavirus infection in patients are expected following delivery of a coronavirus vaccine using the microdose device as were observed following delivery of a coronavirus vaccine using the SOFUSA® DoseConnect™ device as described in Example 3 of the present disclosure.

In some embodiments, lymphatic delivery of a coronavirus vaccine using the microdose devices and methods described herein may result in increased levels of Th1 (e.g. CD4⁺IFNγ⁺) T-cells and Th2 (e.g. CD4⁺ IL-4⁺) T-cells in a patient as compared to other routes of administration such as such as intravenous, subcutaneous, intramuscular, or intradermal injections of the same coronavirus vaccine given to a patient at an equivalent dose. For example, in Example 3 of the present disclosure, lymphatic delivery of rS1-Fc using a SOFUSA® DoseConnect™ device produced significantly higher levels of both Th1 (CD4⁺IFNγ⁺) and Th2 (CD4⁺ IL-4⁺) T-cells, as compared to intramuscular or intradermal injection of rS1-Fc. Furthermore, in some embodiments, lymphatic delivery of a coronavirus vaccine using the microdose devices and methods described herein may provide increased ratio of Th1 response to Th2 response. For example, in Example 3 of the present disclosure, lymphatic delivery of rS1-Fc using a SOFUSA® DoseConnect™ device produced a significant increase in Th1 response as compared to Th2 response, whereas there were no significant differences between the increase in Th1 and Th2 responses following intramuscular or intradermal injections of rS1-Fc vaccine.

In some embodiments, lymphatic delivery of a coronavirus vaccine (e.g. a rS1-Fc vaccine) using the microdose devices and methods described herein may provide up to 1.5-fold, 2-fold, 3-fold, 4-fold, 5-fold, 6-fold, 7-fold, 8-fold, 9-fold, or 10-fold increase in Th1 (e.g. CD4⁺IFNγ⁺) T-cells and/or Th2 (e.g. CD4⁺ IL-4⁺) T-cells in a patient as compared to other routes of administration such as such as intravenous, subcutaneous, intramuscular, or intradermal injections of the same coronavirus vaccine given to a patient at the same dose.

In some embodiments, lymphatic delivery of a coronavirus vaccine (e.g. a rS1-Fc vaccine) using the microdose devices and methods described herein may provide an increased ratio of Th1 response to Th2 response (e.g. an increased ratio of CD4⁺IFNγ⁺ T-cells to CD4⁺ IL-4⁺ T-cells) in a patient (e.g., up to 1.5-fold, 2-fold, 3-fold, 4-fold, 5-fold, 6-fold, 7-fold, 8-fold, 9-fold, or 10-fold increase in the ratio of Th1 response to Th2 response) as compared to the ratio of Th1 response to Th2 response in a patient following delivery of the same coronavirus vaccine given to a patient at the same dose using other routes of administration such as such as intravenous, subcutaneous, intramuscular, or intradermal injections. For example, in some embodiments, lymphatic delivery of a coronavirus vaccine may result in an increase in Th1 T-cells that is up to 1.5-fold, 2-fold, 3-fold, 4-fold, 5-fold, 6-fold, 7-fold, 8-fold, 9-fold, or 10-fold higher than an increase in Th2 T-cells.

In some embodiments, lymphatic delivery of a coronavirus vaccine using the microdose devices and methods described herein may provide increased levels of CD8⁺ (e.g. CD8⁺IFNγ⁺) T-cells in a patient as compared to other routes of administration such as intravenous, subcutaneous, intramuscular, or intradermal injections of the same coronavirus vaccine given to a patient at the samedose. For example, in Example 3 of the present disclosure, lymphatic delivery of rS1-Fc using a SOFUSA® DoseConnect™ device produced significantly higher levels of CD8⁺IFNγ⁺ T-cells than following intramuscular or intradermal injection of rS1-Fc vaccine.

In some embodiments, lymphatic delivery of a coronavirus vaccine (e.g. a rS1-Fc vaccine) using the microdose devices and methods described herein may provide up to 1.5-fold, 2-fold, 3-fold, 4-fold, 5-fold, 6-fold, 7-fold, 8-fold, 9-fold, or 10-fold increase in CD8⁺IFNγ⁺ T-cells in a patient as compared to other routes of administration such as such as intravenous, subcutaneous, intramuscular, or intradermal injections of the same coronavirus vaccine given to a patient at the same dose.

In some embodiments, the therapeutic agent (e.g. the coronavirus vaccine) may be delivered to the patient at a concentration of up to 10 mg/mL, such as up to 10 mg/mL, 9 mg/mL, 8 mg/mL, 7 mg/mL, 6 mg/mL, 5 mg/mL, 4 mg/mL, 3 mg/mL, 2 mg/mL, 1 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, 0.1 mg/mL, 0.05 mg/mL, or 0.01 mg/mL. In some embodiments, the therapeutic agent (e.g. the coronavirus vaccine) may be delivered to the patient at a dose of up to 5 mg in a fluid composition volume of up to 500 μL. In some embodiments, the microdose devices and methods described herein may be used to deliver a fluid composition having a therapeutic agent at a dose of up to 5 mg in a fluid composition volume of up to 500 μL. In some embodiments, the microdose devices and methods described herein may be used to deliver a coronavirus vaccine to a patient at a dose of up to 5 mg in a fluid composition volume of up to 500 μL.

In some embodiments, a coronavirus vaccine may be administered to a patient using any of the devices and methods described in International Patent Application Publication Nos. WO 2014/188343, WO 2014/132239, WO 2014/132240, WO 2013/061208, WO 2012/046149, WO 2011/135531, WO 2011/135530, WO 2011/135533, WO 2014/132240, WO 2015/16821, and International Patent Applications PCT/US2015/028154 (published as WO 2015/168214 A1), PCT/US2015/028150 (published as WO 2015/168210 A1), PCT/US2015/028158 (published as WO2015/168215 A1), PCT/US2015/028162 (published as WO 2015/168217 A1), PCT/US2015/028164 (published as WO 2015/168219 A1), PCT/US2015/038231 (published as WO 2016/003856 A1), PCT/US2015/038232 (published as WO 2016/003857 A1), PCT/US2016/043623 (published as WO 2017/019526 A1), PCT/US2016/043656 (published as WO 2017/019535 A1), PCT/US2017/027879 (published as WO 2017/189258 A1), PCT/US2017/027891 (published as WO 2017/189259 A1), PCT/US2017/064604 (published as WO 2018/111607 A1), PCT/US2017/064609 (published as WO 2018/111609 A1), PCT/US2017/064614 (published as WO 2018/111611 A1), PCT/US2017/064642 (published as WO 2018/111616 A1), PCT/US2017/064657 (published as WO 2018/111620 A1), and PCT/US2017/064668 (published as WO 2018/111621 A1), U.S. Pat. Nos. 9,962,536 and 9,550,053, U.S. application Ser. Nos. 15/305,193, 15/305,206, 15/305,201, 15/744,346, 14/354,223, and International Patent Application No.'s PCT/US2017/027879, PCT/US2017/027891, PCT/US2016/043656, PCT/US2017/064604, PCT/US2017/064609, PCT/US2017/064642, PCT/US2017/064614, PCT/US2017/064657, PCT/US2017/064668, and U.S. Provisional Patent Application No. 62/678,601, filed May 31, 2018, U.S. Provisional Patent Application No. 62/678,592, filed May 31, 2018, and U.S. Provisional Patent Application No. 62/678,584, filed May 31, 2018, and International Application No. PCT/US2019/034736, all of which are incorporated by reference herein in their entirety. Such devices include the SOFUSA® drug delivery platform (Sorrento Therapeutics, Inc., San Diego). For example, such devices include SOFUSA® DoseConnect™ devices (Sorrento Therapeutics, Inc., San Diego).

### EXAMPLES

The devices and methods herein disclosed are further illustrated in the following examples, which are provided by way of illustration and are not intended to be limiting.

### Example 1. Lymphatic Delivery of Indocyanine Green (ICG)

This Example describes experiments to verify delivery of a fluid composition using a microdose device as described herein coupled to a syringe. The example microdose device used in this Example has a 4×4 microneedle array.

C57BL/6 mice were anesthetized with isofluorane and the dorsal region was shaved and covered with depilatory cream (Nair Sensitive) for 8 minutes. The cream was then wiped off with warm, wet paper towels, followed by alcohol wipes.

A syringe was filled with 0.5 mg/ml indocyanine green (ICG) and the syringe was coupled to a microdose device as described herein via the syringe connection assembly of the microdose device as described herein.

While a user held the microdose device coupled to the syringe freely in their hand, the microdose device was placed onto the skin of an anesthetized mouse such that the microneedles penetrated the surface of the skin. 50 μL of IGC was injected for about 2 minutes by manually depressing the plunger of the syringe while the microneedles were below the surface of the skin of the mouse.

Lymphatic imaging was performed using non-invasive near-infrared fluorescence (NIRF) imaging as described in Sevick-Muraca E M, Kwon S, Rasmussen J C. Emerging lymphatic imaging technologies for mouse and man. J Clin Invest. 2014; 124:905-14.

Fluorescence imaging of IGC in lymph nodes was observed about 20 seconds post-injection, as shown in FIG. 11.

### Example 2. Verification of Lymphatic Delivery of Indocyanine Green (ICG) Before Lymphatic Administration of rS1-Fc Vaccine Using a SOFUSA® DoseConnect™ Device

This example describes experiments to verify lymph node delivery with ICG prior to rS1-Fc vaccine injection in C57BL/6 mice (average weight 26 g).

ICG was first administered to verify delivery to the right brachial lymph node before switching over to the rS1-Fc vaccine (see Example 3).

A SOFUSA® DoseConnect™ device with a 10×10 microneedle array was used in this Example.

Twenty-four hours prior to SOFUSA® DoseConnect™ administration, mice were anesthetized with isofluorane and the dorsal region was shaved and covered with depilatory cream (Nair Sensitive) for 8 minutes. The cream was then wiped off with warm, wet paper towels, followed by alcohol wipes. SOFUSA® DoseConnect™ was then applied to the dorsal region using a plastic shell with a skin adhesive. A hand-held applicator was then placed over the plastic shell to insert the microneedles into the skin. The operation of the device was as follows. The applicator strikes the microneedles with a post traveling at a velocity of 6 m/s. There is a total of 100 microneedles over the area of 66 mm². With the microneedles inserted in the skin, the syringe pump was started to deliver indocyanine green (ICG).

0.5 mg/ml ICG was infused at a rate of 75 μl per hour on the right dorso-lateral side of isoflurane anesthetized healthy mice. Lymphatic imaging was performed using non-invasive near-infrared fluorescence (NIRF) imaging as described in Sevick-Muraca E M, Kwon S, Rasmussen J C. Emerging lymphatic imaging technologies for mouse and man. J Clin Invest. 2014; 124:905-14.

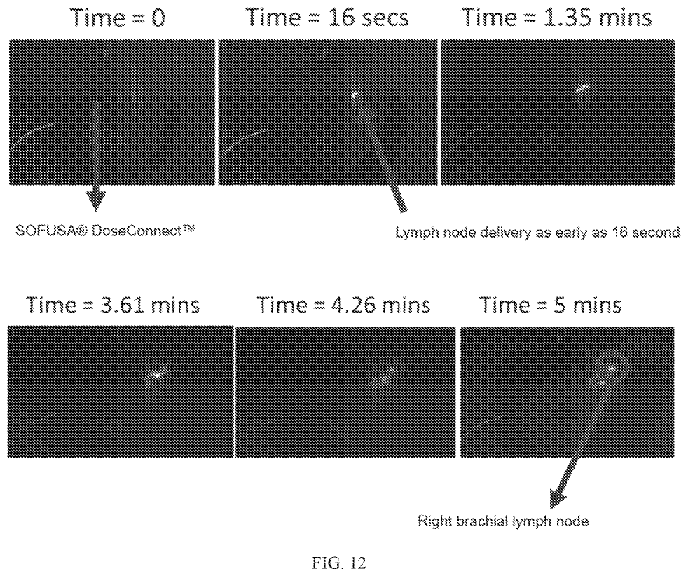

As shown in FIG. 12, lymph node delivery was verified, and observed as early as 16 seconds after initiation of administration of ICG via SOFUSA® DoseConnect™.

### Example 3. IgG Response and T-Cell Response Following Lymphatic, Intramuscular, or Intradermal Administration of rS1-Fc Vaccine

This Example describes experiments providing pre-clinical data in mice following lymphatic delivery of a 100 μg dose of an example coronavirus vaccine, rS1-Fc vaccine (Sorrento Therapeutics, Inc.) using a SOFUSA® DoseConnect™ device with a 10×10 microneedle array.

Following verification of lymphatic delivery using ICG (see Example 2), the same SOFUSA® DoseConnect™ device was left in position with the microneedles inserted in the skin of the mice and used to administer 100 μg of the rS1-Fc vaccine (2 mg/mL in sterile water) at 75 μL per hour over 40 minutes.

The data are compared to results obtained following intramuscular or intradermal injection of mice with 100 μg of the rS1-Fc vaccine. Intradermal injection were performed using multiple 5-10 Mantoux injections proximal to brachial lymph nodes.

Administration to mice of 100 μg of the rS1-Fc vaccine via SOFUSA® DoseConnect™ was performed at day 0 (first immunization) and day 21 (second immunization) and serum was collected 3 days before the first immunization and on day 3, 7, 10, 14, 21, 28, 35 and 42 days following the first immunization.

Administration to mice of 100 μg of the rS1-Fc vaccine via intramuscular injection was performed at day 0 (first immunization) and day 21 (second immunization) and serum was collected 3 days before the first immunization and on day 3, 7, 10, 14, 17, 24, 28, 31, 35 and 47 days following the first immunization.

Administration to mice of 100 μg of the rS1-Fc vaccine via intradermal injection was performed at day 0 (first immunization) and day 21 (second immunization) and serum was collected 3 days before the first immunization and on day 3, 4, 7, 11, 14, 18, 25, 28, 32, 35, 39, and 41 days following the first immunization.

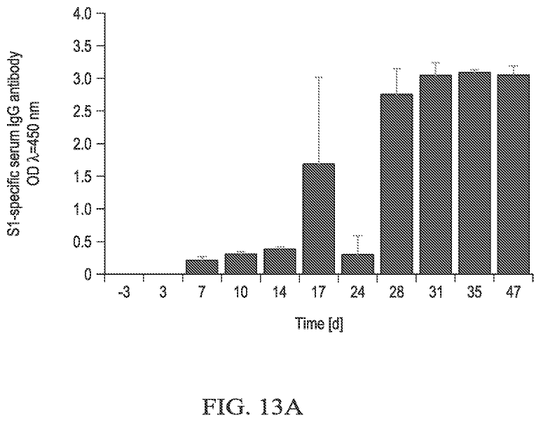

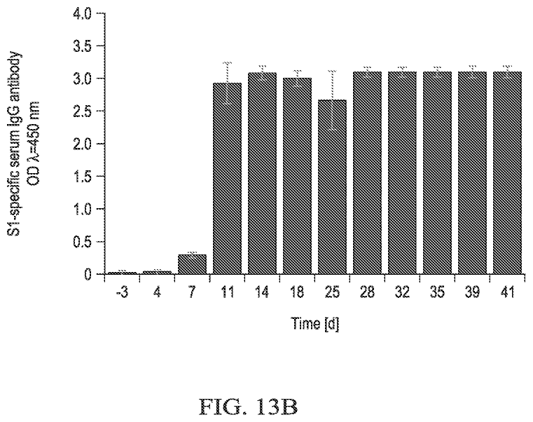

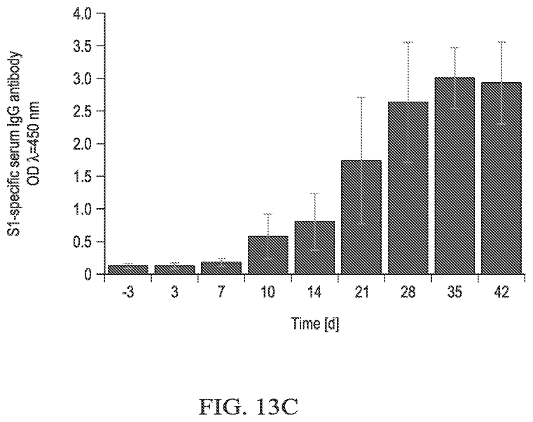

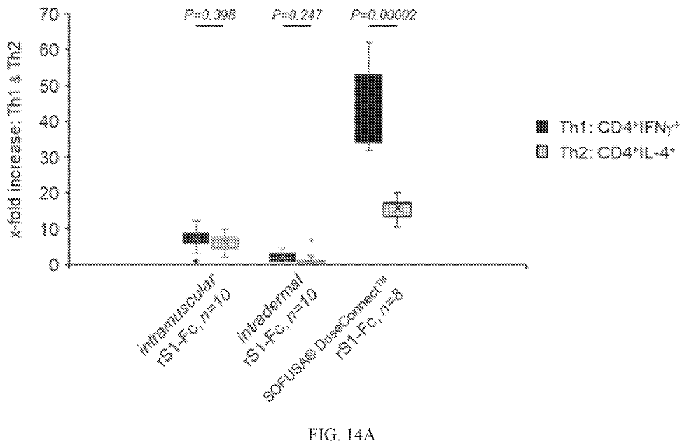

S1-specific serum IgG antibody optical density (OD, 450 nm) was determined at each of the serum collection time points. Results of the IgG response following administration of 100 μg of the rS1-Fc vaccine via SOFUSA® DoseConnect™ or via intramuscular or intradermal injection are shown in FIG. 13A-13C. Similar levels of S1-specific serum IgG antibody were detected by about 28 days after the first immunization by SOFUSA® DoseConnect™ or via intramuscular or intradermal injection.

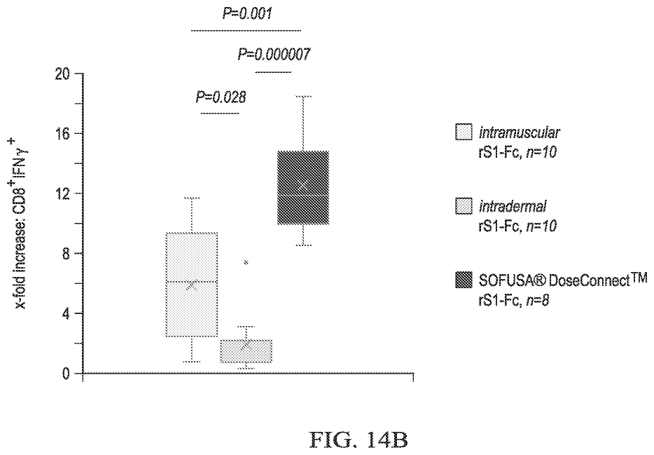

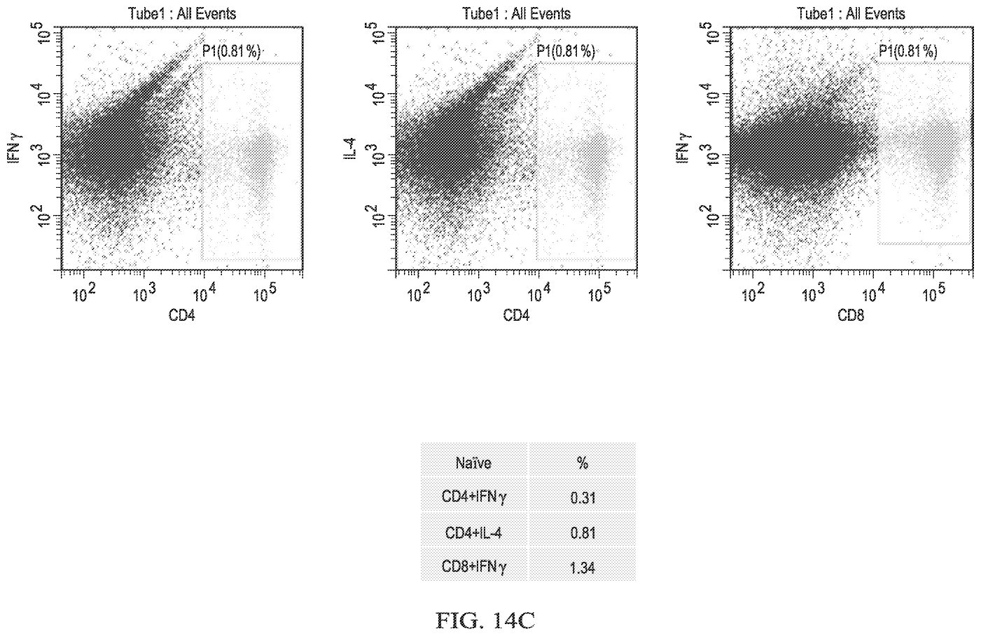

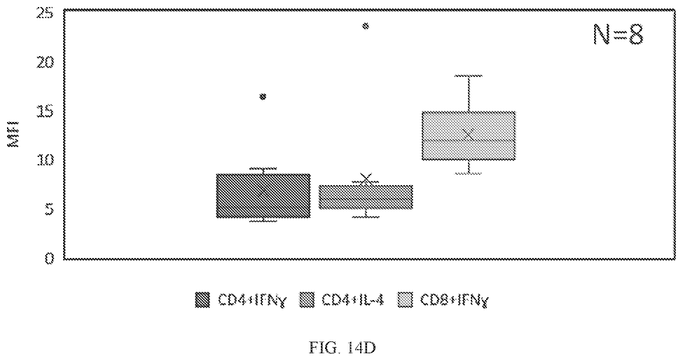

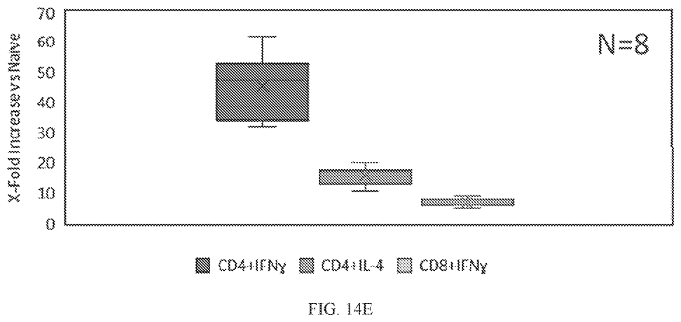

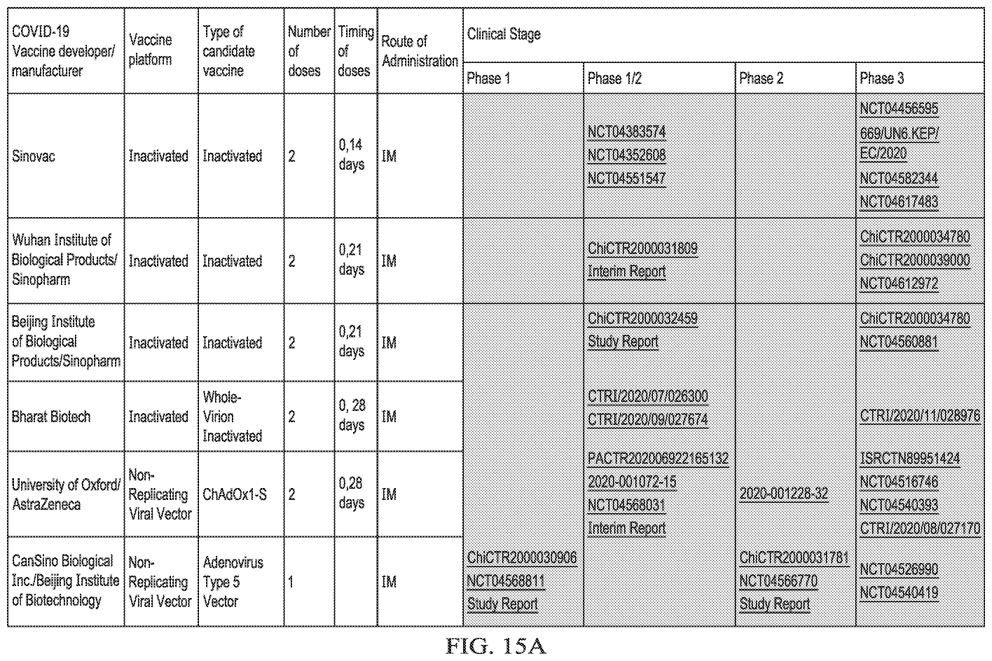

FIG. 14A is a graph showing box and whisker plots reporting fold increases in T-cell responses (Th1 and Th2) in mice following lymphatic administration of rS1-Fc vaccine via SOFUSA® DoseConnect™, or intramuscular or intradermal injection. Administration of rS1-Fc vaccine via SOFUSA® DoseConnect™ produced the largest increase in both Th1 (CD4⁺IFNγ⁺) and Th2 (CD4⁺ IL-4⁺) T-cells, with an average 45.3-fold increase and an average 15.86-fold increase respectively, and also produced a significant increase in Th1 response as compared to Th2 response (p=00002). In comparison, rS1-Fc vaccine intramuscular or intradermal injections produced less than 10-fold increase in both Th1 and Th2 responses, and there were no significant differences between the increase in Th1 and Th2 responses following intramuscular or intradermal injections of rS1-Fc vaccine.

FIG. 14B is a graph showing box and whisker plots reporting example fold increases in T-cell responses (CD8⁺IFNγ⁺) in mice following administration of rS1-Fc vaccine via SOFUSA® DoseConnect™, or intramuscular or intradermal injection. Administration of rS1-Fc vaccine via SOFUSA® DoseConnect™ produced the largest increase in CD8⁺IFNγ⁺ T-cells, with an approximate average 13-fold increase. This increase was significantly larger than an approximately 7-fold increase in CD8⁺IFNγ⁺ T-cells following intramuscular injection of rS1-Fc vaccine (p=0.001) and also significantly larger than an approximately 2-fold increase in CD8⁺IFNγ⁺ T-cells following intradermal injection of rS1-Fc vaccine (p=0.000007).

FIG. 14C is graphs reporting example results of flow cytometry analysis of T-cells from plasma of mice following administration of rS1-Fc vaccine via SOFUSA® DoseConnect™ The average number of live cells in the samples was 86% The graph on the left shows an example of flow cytometry results quantifying Th1 (CD4⁺IFNγ⁺) T-cells, while the graph in the middle shows an example of flow cytometry results quantifying Th2 (CD4⁺ IL-4⁺) T-cells, and the graph on the right shows an example of flow cytometry results quantifying CD8⁺IFNγ⁺ T-cells. In serum from naïve mice before administration of rS1-Fc vaccine via SOFUSA® DoseConnect™, the percentage of CD4⁺IFNγ⁺ T-cells was 0.31%, the percentage of CD4⁺ IL-4⁺ T-cells was 0.81%, and the percentage of CD8⁺IFNγ⁺ T-cells was 1.34%.

FIG. 14D is a graph reporting example Mean Fluorescence Intensity (MFI) quantification by flow cytometry of CD4⁺IFNγ⁺ T-cells, CD4⁺ IL-4 T-cells and CD8⁺IFNγ⁺ T-cells from plasma of mice following administration of rS1-Fc vaccine via SOFUSA® DoseConnect™. FIG. 14E is a graph showing box and whisker plots reporting example fold increases in Mean Fluorescence Intensity (MFI) quantification by flow cytometry of CD4⁺IFNγ⁺ T-cells, CD4⁺ IL-4 T-cells and CD8⁺IFNγ⁺ T-cells from plasma of mice following administration of rS1-Fc vaccine via SOFUSA® DoseConnect™ as compared to Mean Fluorescence Intensity (MFI) quantification by flow cytometry of CD4⁺IFNγ⁺ T-cells, CD4⁺ IL-4 T-cells and CD8⁺IFNγ⁺ T-cells from plasma of naïve mice before administration of rS1-Fc vaccine via SOFUSA® DoseConnect™.

### Example 4. Leak Testing of Microdose Device

This Example described results of testing fluid delivery using a microdose device over a range of fluid flow rates and assessment of whether leaks were present using a fluorescent dye present in the fluid.

An example microdose device having a 4×4 microneedle array, having 16 microneedles in total, was coupled to a syringe and the syringe attached to a syringe pump set to inject the fluid from the syringe through the microdose device at the flow rates shown in Table 1 (showing the total flow rate and also the flow rate through each of the 16 microneedles of the example microdose device). At each flow rate, a force transducer measured the pressure of the fluid flowing through the microdose device. The microdose device was also visually inspected for leaks.

TABLE 1
Results of leak testing of the microdose device.

| Flow rate | Microneedle flow rate | Pressure | Leak or failure |
|---|---|---|---|
| (μL/hour) | (μL/hour) | (psi) | (Yes or No) |
| 100 | 6.25 | 5.3 | No |
| 250 | 15.6 | 11.2 | No |
| 500 | 31.25 | 20 | No |
| 1000 | 62.5 | 48 | No |
| 2000 | 125 | 82 | No |

The example microdose device was able to withstand at least 82 psi with no leaks, while delivering fluid at a rate of at least 2000 μL/hour, which equates to delivering fluid at a rate of at least approximately 33 μL/minute.

### Example 5. Microneedle Skin Penetration Testing

This Example describes experiments to assess microneedle penetration into skin using hand-held, manual insertion with example microdose devices. Pig cadaver skin was used for quantifying the depth of microneedle insertion into skin. Manual insertion by hand was conducted using example microdose devices having a 4×4 microneedle array (the microneedle array having 16 microneedles in total). Analysis of microneedle insertion depth was performed by visual assessment of microneedle insertion on methylene blue dyed skin, with the skin shaved at 10 μm increments and visual assessment performed at each depth.

Tests were performed using microdose devices having three different configurations of proudness of the distal face of the base plate of the microneedle array protruding axially from the distal face of the plenum by 0.56, 1.06, or 1.21 μm. Representative results are shown in Table 2.

TABLE 2
Results of microneedle penetration in skin.

Proudness of distal face of base
plate of microneedle array (μm)

| | 0.56 | 1.06 | 1.21 |
|---|---|---|---|
| Microneedle penetration | 158 ± 29 | 196 ± 89 | 179 ± 40 |
| into skin (μm), Sample 1 | | | |
| Microneedle penetration | 241 ± 62 | 198 ± 87 | 159 ± 42 |
| into skin (μm), Sample 2 | | | |
| Microneedle penetration | 154 ± 44 | 183 ± 49 | 189 ± 41 |
| into skin (μm), Sample 3 | | | |

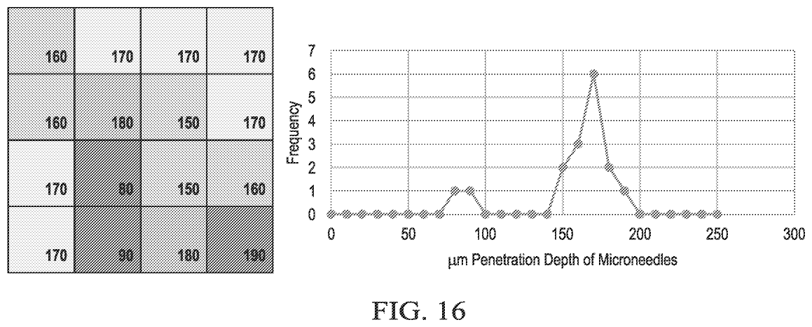

FIG. 16 shows an example schematic of microneedle skin penetration depth for each microneedle of an example 4×4 microneedle array (left image) and a graph reporting frequency distribution the example microneedle penetration results for the image shown. For the example results shown in FIG. 16, the maximum microneedle skin penetration depth is 190 μm, the minimum microneedle skin penetration depth is 80 μm, and the average is 157.5 μm with a standard deviation of 29.26 μm.

These example results are consistent with those observed for SOFUSA® DoseConnect™ devices having e.g. 10×10 or 18×18 microneedle arrays (having 100 or 324 microneedles respectively) with skin penetration using an applicator. The results in this Example shown that manual, hand-held insertion of an example microdose device of the present disclosure having a 4×4 microneedle array provides suitable microneedle skin penetration for lymphatic delivery of fluid compositions in a patient.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. When particular values or compositions are provided in the instant disclosure, unless otherwise stated, the meaning of “about” or “approximately” should be assumed to be within an acceptable error range for that particular value or composition.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A device for delivering a fluidic composition across a dermal barrier of a patient, the device comprising:
a microneedle fluidic block assembly, comprising:
a microneedle array comprising a plurality of microneedles disposed on a distal face of a base plate, wherein the microneedles have a fluidic exit channel defined therein, the microneedles capable of penetrating the stratum comeum of the skin of a patient and delivering a fluidic composition to a depth below the surface of the skin of the patient;
a fluidic distribution block having a distal face coupled to a proximal face of the base plate of the microneedle array, the fluidic distribution block comprising a fluid distribution manifold defined therein and configured to be fluidically connected with the fluidic exit channels of the microneedles and to controllably distribute the fluidic composition to the plurality of microneedles through the fluidic exit channels; and
a syringe connection assembly having a fluidic path defined therein, the syringe connection assembly comprising:
a distal end coupled to a proximal face of the fluidic distribution block, the fluidic path of the syringe connection assembly fluidically connected to the fluid distribution manifold; and
a proximal end configured to be coupled to a syringe barrel having a bore defined therein, the fluidic path of the syringe connection assembly configured to be fluidically connected to the bore of the syringe barrel.

2. The device of claim 1, wherein the syringe connection assembly comprises a plenum coupled to and fluidically connected with a tubing connector; wherein the tubing connector has:
a distal portion coupled to a proximal face of the plenum; and
a proximal portion configured to be fluidically connected to the bore of the syringe barrel; and
the plenum has:
a distal face coupled to the proximal face of the fluidic distribution block, and fluidically connected to the fluid distribution manifold.

3. The device of claim 1, further comprising a first gasket disposed between and coupled to the distal end of the syringe connection assembly and the proximal face of the fluidic distribution block; wherein the first gasket has a hole in fluidic connection with the fluidic path of the syringe connection assembly and the fluid distribution manifold.

4. The device of claim 3, wherein the first gasket has a proximal face and a distal face, wherein the proximal face and the distal face has an adhesive layer disposed thereon and adapted to adhere the distal end of the syringe connection assembly to the proximal face of the fluidic distribution block.

5. The device of claim 1, wherein the fluid distribution manifold is configured to provide a substantially equal flow rate of the fluidic composition to the exit channels in each microneedle.

6. The device of claim 1, wherein the fluid distribution manifold comprises:
a proximal entrance disposed within the proximal face of the fluidic distribution block and in fluidic connection with the distal end of the syringe connection assembly; and
supply channels fluidically connected to the proximal entrance and configured to distribute a fluidic composition to a plurality of resistance channels;
wherein the plurality of resistance channels fluidically connected to the supply channels and configured to provide a resistance to flow of the fluidic composition; and
a plurality of outlet apertures, each outlet aperture fluidically connected to a resistance channel and a fluidic exit channel.

7. The device of claim 6, wherein the fluidic distribution block comprises a proximal portion having a distal face coupled to a proximal face of a distal portion, wherein the supply channels and the resistance channels are disposed on the distal face of the proximal portion and/or the proximal face of the distal portion.

8. The device of claim 7, wherein the fluidic distribution block comprises a polymer material, a glass material and/or a silicon material, and the fluid distribution manifold is formed therein by a drilling method, a cutting method, a powder blasting method, and/or an etching method.

9. The device of claim 7, wherein the proximal portion and the distal portion are bonded together.

10. The device of claim 6, wherein the resistance channels have:
a length of from 400 μm to 1,000 μm;
an axial depth of from 10 μm to about 20 μm; and
a lateral width of from 15 μm to 70 μm.

11. The device of claim 1, wherein the plurality of microneedles is from 2 to 100 microneedles.

12. The device of claim 6, wherein:
each of the resistance channels includes one or more inlet apertures adapted to be in fluidic connection with the supply channel;
the resistance channels comprise inner resistance channels located proximal to a lateral center of the fluidic distribution block, and outer resistance channels located distal to the lateral center of the fluidic distribution block;
wherein two or more inner resistance channels are in fluidic connection with one inlet aperture; and
each outer resistance channel is in fluidic connection with one inlet aperture.

13. The device of claim 1, further comprising a protective cap coupled to the distal end of the syringe connection assembly and configured to protect the physical integrity and/or sterility of the microneedle fluidic block assembly.

14. The device of claim 13, wherein the protective cap is configured to be slidably coupled to the syringe connection assembly.

15. The device of claim 1, further comprising a syringe including a barrel, wherein the proximal end of the syringe connection assembly is coupled to the syringe barrel and fluidically connected to the bore of the syringe barrel.

16. The device of claim 15, wherein the bore has a longitudinal axis, the syringe further comprising a plunger slidably disposed within the longitudinal axis of the bore, the syringe adapted to eject a volume of from 1 μl to 500 μl of a fluidic composition disposed within the bore in response to an axial force applied to the plunger.

17. The device of claim 16, wherein the syringe is adapted to eject the volume of the fluidic composition over a period of time from 0.1 second to 300 seconds.

18. The device of claim 16, wherein the syringe further comprises a fluidic composition disposed within the bore.

19. The device of claim 18, wherein in response to an axial force applied to the plunger, the device is adapted to deliver the fluidic composition to a patient through the exit channels of the plurality of microneedles.

20. The device of claim 19, wherein the device is adapted to be manually operable by a user, wherein the axial force is applied by the hand of the user.

21.-46. (canceled)
